(19) United States
(12) Patent Application Publication
(10) Pub. No.: US 2006/0223178 A1
(43) Pub. Date: Oct. 5, 2006

(54) Devices and methods for magnetic enrichment of cells and other particles

(76) Inventors: Tom Barber, Cambridge, MA (US); Lotien Richard Huang, Brookline, MA (US); Martin Schmidt, Reading, MA (US); Mehmet Toner, Wellesley, MA (US); Ravi Kapur, Stoughton, MA (US)

Correspondence Address: CLARK & ELBING LLP, 101 FEDERAL STREET, BOSTON, MA, 02110, US

(21) Appl. No.: 11/323,971

(22) Filed: Dec. 29, 2005

Related U.S. Application Data
(63) Continuation-in-part of application No. 11/227,904, filed on Sep. 15, 2005.
(60) Provisional application No. 60/668,415, filed on Apr. 5, 2005.
(60) Provisional application No. 60/704,067, filed on Jul. 29, 2005.

(51) Int. Cl. C12N 5/08; C12N 5/00

(52) U.S. Cl. 435/325; 435/372

(57) ABSTRACT

The invention features devices and methods for the enrichment of cells and other desired analytes by employing a magnetic field, alone or in conjunction with size-based separation. The devices and methods may be advantageously employed to enrich for rare cells, e.g., fetal cells or epithelial cells, present in a sample, e.g., maternal blood.

### CROSS-REFERENCE TO RELATED APPLICATIONS

This application is a continuation-in-part of U.S. application Ser. No. 11/227,904, filed Sep. 15, 2005, which claims the benefit of U.S. Provisional Application Ser. Nos. 60/668,415, filed Apr. 5, 2005 and 60/704,067, filed Jul. 29, 2005, each of which is hereby incorporated by reference in its entirety.

### BACKGROUND OF THE INVENTION

The invention relates to the fields of cell separation, medical diagnostics, and microfluidic devices.

Clinically or environmentally relevant information may often be present in a sample, but in quantities too low to detect. Thus, various enrichment or amplification methods are often employed in order to increase the detectability of such information.

For cells, different flow cytometry and cell sorting methods are available, but these techniques typically employ large and expensive pieces of equipment, which require large volumes of sample and skilled operators. These cytometers and sorters use methods like electrostatic deflection, centrifugation, fluorescence activated cell sorting (FACS), and magnetic activated cell sorting (MACS) to achieve cell separation. These methods often suffer from the inability to enrich a sample sufficiently to allow analysis of rare components of the sample. Furthermore, such techniques may result in unacceptable losses of such rare components, e.g., through inefficient separation or degradation of the components.

Thus, there is a need for new devices and methods for enriching samples.

### SUMMARY OF THE INVENTION

In general, the invention features devices and methods that allow for the enrichment of cells, and other analytes of interest, using magnetic properties, typically in conjunction with another dimension of separation, e.g., size, shape, deformability, or affinity. Preferably, analytes of interest are separated based on intrinsic magnetic properties, which may be altered as described herein.

Accordingly, the invention features a device for producing a sample enriched in a first cell or component thereof relative to a second component including a channel through which the first cell or component flows; and a magnet that produces a magnetic field of between 0.05 and 5.0 Tesla and a magnetic field gradient of between 100 Tesla/m and 1,000,000 Tesla/m in the channel. The first cell or component may be retained in the channel, and the second component may not be retained in the channel, or vice versa. The channel may include first and second outlets, where the first cell or component thereof is directed into the first outlet, while the second component is directed into the second outlet. The device may also include pump capable of producing a flow rate of greater than 50,000 cells or components thereof flowing into the channel per second.

The device may further include an analytical module that enriches the first cell or component based on size, shape, deformability, or affinity. The analytical module includes, for example, a first channel having a structure that deterministically deflects particles having a hydrodynamic size above a critical size in a direction not parallel to the average direction of flow in the structure. The structure may include an array of obstacles that form a network of gaps, where a fluid passing through the gaps is divided unequally into a major flux and a minor flux so that the average direction of the major flux is not parallel to the average direction of fluidic flow in the channel. The array of obstacles may include first and second rows, where the second row is displaced laterally relative to the first row so that fluid passing through a gap in the first row is divided unequally into two gaps in the second row.

The device may also include a reagent capable of altering a magnetic property of the first cell or component or second component. The reagent, for example, alters the magnetic properties of a protein, e.g., containing iron, such as fetal hemoglobin, adult hemoglobin, methemoglobin, myoglobin, or a cytochrome, present in the first cell or component or the second component. Exemplary reagents include sodium nitrite, carbon dioxide, oxygen, carbon monoxide, and nitrogen. The reagent may also cause expression or overexpression of a protein that is magnetic in the first cell or component or the second component. For example, the reagent is capable of transfecting the first cell or the second component with a magnetically responsive protein. The reagent may also include a magnetic particle that binds to or is incorporated into the first cell or component or the second component.

The first cell is, for example, a blood cell (e.g., an adult nucleated red blood cell or a fetal nucleated red blood cell, such as from a fetus of less than 10 weeks of age), a nucleated cell, or an enucleated cell. The first cell may be mammalian, avian, reptilian, or amphibian. Exemplary components of the first cell include nuclei, peri-nuclear compartments, nuclear membranes, mitochondria, chloroplasts, or cell membranes, lipids, polysaccharides, proteins, nucleic acids, viral particles, and ribosomes.

In preferred embodiment, at least 90% of the first cell or component is retained in the device and at least 90% of the second component is not retained in the device.

In another aspect, the device of the invention is used to produce a sample enriched in a first cell or component thereof relative to a second component by introducing a sample including the first cell or component into the channel and allowing the passage of the first cell or component or the second component relative to the other to be altered based on a magnetic property, thereby producing the sample enriched in the first cell or component. The sample introduced into the device may be enriched for the first cell or component relative to a third component. For example, the sample may be contacted with an analytical module that enriches the first cell or component relative to the third component based on size, shape, deformability, or affinity. An exemplary analytical module includes a first channel having a structure that deterministically deflects particles having a hydrodynamic size above a critical size in a direction not parallel to the average direction of flow in the structure, wherein the particles are the first cell or component or are the third component of the sample. The sample enriched in the first cell or component may retain at least 70% of the first cells or components present in the sample. The sample enriched in the first cell or component is, for example, enriched by a factor of 100. The method may further include contacting the sample with a reagent capable of altering a magnetic property of the first cell or component or second component. The sample enriched in the first cell or component may include at least 90% of the first cell or component in the sample introduced prior to enrichment and less than 10% of the second component in the sample prior to enrichment. Exemplary reagents, first cells, components thereof, second components, purities, and flow rates are described herein.

The invention further features an alternative method of producing a sample enriched in a first cell or component thereof relative to a second component by contacting a sample potentially including the first cell or component with a reagent, as described herein, that alters the magnetic properties of a protein expressed in the first cell or component or the second component of the sample to produce an altered sample; contacting the altered sample with a channel having a magnet positioned relative to the channel and producing a magnetic field and magnetic field gradient capable of altering the passage of the first cell or component or the second component relative to the other, thereby producing the sample enriched in the first cell or component. In certain embodiments, the sample is enriched for the first cell or component relative to a third component prior to contacting the sample with the magnetic field. For example, the sample may be contacted with an analytical module that enriches the first cell or component relative to the third component based on size, shape, deformability, or affinity. An exemplary analytical module includes a first channel having a structure that deterministically deflects particles having a hydrodynamic size above a critical size in a direction not parallel to the average direction of flow in the structure, wherein the particles are the first cell or component or are the third component of the sample. Exemplary structures are described herein. The sample enriched in the first cell or component may retain at least 70% of the first cells or components present in the sample. The sample enriched in the first cell or component is, for example, enriched by a factor of 100. The sample enriched in the first cell or component may include at least 90% of the first cell or component in the sample introduced prior to enrichment and less than 10% of the second component in the sample prior to enrichment. Exemplary reagents, first cells, components thereof, second components, purities, and flow rates are described herein.

In another aspect, the invention features a method for enriching a first analyte from a fluid sample (e.g., a blood sample, such as a maternal blood sample) relative to second and third analytes by performing a first enrichment step to enrich the first analyte from the fluid sample based on hydrodynamic size using a plurality of obstacles that direct the first analyte in a first direction and the second analyte in a second direction, and performing a second enrichment step to enrich the first analyte from the fluid sample based on an intrinsic or extrinsic magnetic property of the first or third analyte. Exemplary first analytes are cells, as described herein. The second enrichment step may include applying a magnetic field to the product of the first enrichment step. The magnetic field may attract or repulse the first or third analyte. Typically, the magnetic field alters the passage of the first analyte relative to the third analyte. The method may further include the step of altering a magnetic property of, e.g., by deoxygenating, the first enrichment product. Deoxygenating step may include contacting the product of the first enrichment step with CO, CO₂, N₂, or NaNO₂. The method may also include paramagnetizing or diamagnetizing the first or third analyte. The first enrichment step and the second enrichment step may occur in series. In certain embodiments, the first enrichment step or the second enrichment step includes a plurality of enrichment steps that occur in series or in parallel to one another. The first or second enrichment step occurs during sample flow through. The second enrichment step may be based on an intrinsic or extrinsic magnetic property. Preferably, greater than 50,000 analytes are subjected to enrichment per second. Exemplary magnetic fields and magnetic field gradients are described herein.

The invention further features a system including a first module having an array of obstacles that selectively directs one or more first analytes having a hydrodynamic size greater than a critical size in a first direction towards a first outlet and one or more second analytes having a hydrodynamic size smaller than the critical size in a second direction towards a second outlet; a second module having a channel for receiving the first analytes from the first outlet; and a magnet that generates a magnetic field and magnetic field gradient in the channel to alter passage of the first analytes.

The invention also features a system including a flow-through channel having a two dimensional array of obstacles that selectively directs one or more first analytes having a hydrodynamic size greater than a critical size in a first direction towards a first outlet and one or more second analytes having a hydrodynamic size less than a critical size in a second direction towards a second outlet; and a magnet that generates a magnetic field and magnetic field gradient to alter the passage of the first analytes.

The first analyte is, for example, a nucleated red blood cell, e.g., a fetal nucleated red blood cell, and the second analyte is, for example, an enucleated red blood cell. The first analyte includes, for example, fetal hemoglobin, adult hemoglobin, methemoglobin, myoglobin, or a cytochrome. The systems may also include a reservoir containing a deoxygenating agent, or other reagent capable of altering a magnetic property, coupled to the array of obstacles or the channel. The systems may further include a reservoir containing a probe, e.g., a nucleic acid probe or an antibody probe, for specifically binding the first analyte or a component thereof. The passage of the first analyte is altered, for example, based on an intrinsic or extrinsic magnetic property. An exemplary magnetic field strength for use in the systems is between 0.5 and 5.0 Tesla, and an exemplary magnetic field gradient is between 100 Tesla/m and 1,000,000 Tesla/m. The systems may also include pump capable of producing a flow rate of greater than 50,000 cells or components thereof flowing into the channel per second.

By “analyte” is meant a molecule, other chemical species, e.g., an ion, or particle. Exemplary analytes include cells, viruses, nucleic acids, proteins, carbohydrates, and small organic molecules.

By “biological particle” is meant any species of biological origin that is insoluble in aqueous media on the time scale of sample acquisition, preparation, storage, and analysis. Examples include cells, particulate cell components, viruses, and complexes including proteins, lipids, nucleic acids, and carbohydrates.

By “biological sample” is meant any sample of biological origin or containing, or potentially containing, biological particles. Preferred biological samples are cellular samples.

By “blood component” is meant any component of whole blood, including host red blood cells, white blood cells, and platelets. Blood components also include the components of plasma, e.g., proteins, lipids, nucleic acids, and carbohydrates, and any other cells that may be present in blood, e.g., because of current or past pregnancy, organ transplant, or infection.

By “cellular sample” is meant a sample containing cells or components thereof. Such samples include naturally occurring fluids (e.g., blood, lymph, cerebrospinal fluid, urine, cervical lavage, and water samples), portions of such fluids, and fluids into which cells have been introduced (e.g., culture media, and liquefied tissue samples). The term also includes a lysate.

By “capture moiety” is meant a chemical species to which an analyte binds. A capture moiety may be a compound coupled to a surface or the material making up the surface. Exemplary capture moieties include antibodies, oligo- or polypeptides, nucleic acids, other proteins, synthetic polymers, and carbohydrates.

By “channel” is meant a gap through which fluid may flow. A channel may be a capillary, a conduit, or a strip of hydrophilic pattern on an otherwise hydrophobic surface wherein aqueous fluids are confined.

By “component” of cell is meant any component of a cell that may be at least partially isolated upon lysis of the cell. Cellular components may be organelles (e.g., nuclei, peri-nuclear compartments, nuclear membranes, mitochondria, chloroplasts, or cell membranes), polymers or molecular complexes (e.g., lipids, polysaccharides, proteins (membrane, trans-membrane, or cytosolic), nucleic acids (native, therapeutic, or pathogenic), viral particles, or ribosomes), or other molecules (e.g., hormones, ions, cofactors, or drugs). By “component” of a cellular sample is meant a subset of cells contained within the sample.

By “enriched sample” is meant a sample containing an analyte that has been processed to increase the relative amount of the analyte relative to other analytes typically present in a sample. For example, samples may be enriched by increasing the amount of the analyte of interest by at least 10%, 25%, 50%, 75%, 100% or by a factor of at least 1000, 10,000, 100,000, or 1,000,000.

By “depleted sample” is meant a sample containing an analyte that has been processed to decrease the amount of the analyte relative to other analytes typically present in a sample. For example, samples may be depleted by decreasing the amount of the analyte of interest by at least 5%, 10%, 25%, 50%, 75%, 90%, 95%, 97%, 98%, 99%, or even 100%.

By “exchange buffer” in the context of a sample (e.g., a cellular sample) is meant a medium distinct from the medium in which the sample is originally suspended, and into which one or more components of the sample are to be exchanged.

By “extrinsic magnetic property” of an analyte is meant a magnetic property that is not endogenous to the analyte.

By “flow-extracting boundary” is meant a boundary designed to remove fluid from an array.

By “flow-feeding boundary” is meant a boundary designed to add fluid to an array.

By “gap” is meant an opening through which fluids and/or particles may flow. For example, a gap may be a capillary, a space between two obstacles wherein fluids may flow, or a hydrophilic pattern on an otherwise hydrophobic surface wherein aqueous fluids are confined. In a preferred embodiment of the invention, the network of gaps is defined by an array of obstacles. In this embodiment, the gaps are the spaces between adjacent obstacles. In a preferred embodiment, the network of gaps is constructed with an array of obstacles on the surface of a substrate.

By “hydrodynamic size” is meant the effective size of a particle when interacting with a flow, obstacles (e.g., posts), or other particles. The obstacles or other particles may be in a microfluidic structure. It is used as a general term for particle volume, shape, and deformability in the flow.

By “intracellular activation” is meant activation of second messenger pathways, leading to transcription factor activation, or activation of kinases or other metabolic pathways. Intracellular activation through modulation of external cell membrane antigens can also lead to changes in receptor trafficking.

By “intrinsic magnetic property” of an analyte is meant a magnetic property that is endogenous to the analyte. An intrinsic magnetic property may be present at the beginning of an assay, or it may be induced in the analyte by a suitable reagent. Exemplary intrinsic magnetic properties include those imparted by an iron-containing protein expressed by a cell.

By “labeling reagent” is meant a reagent that is capable of binding to an analyte, being internalized or otherwise absorbed, and being detected, e.g., through shape, morphology, color, fluorescence, luminescence, phosphorescence, absorbance, magnetic properties, or radioactive emission.

By “metabolome” is meant the set of compounds within a cell, other than proteins and nucleic acids, that participate in metabolic reactions and that are required for the maintenance, growth or normal function of a cell.

By “microfluidic” is meant having at least one dimension of less than 1 mm.

By “obstacle” is meant an impediment to flow in a channel, e.g., a protrusion from one surface. For example, an obstacle may refer to a post outstanding on a base substrate or a hydrophobic barrier for aqueous fluids. In some embodiments, the obstacle may be partially permeable. For example, an obstacle may be a post made of porous material, wherein the pores allow penetration of an aqueous component but are too small for the particles being separated to enter.

By “shrinking reagent” is meant a reagent that decreases the hydrodynamic size of a particle. Shrinking reagents may act by decreasing the volume, increasing the deformability, or changing the shape of a particle.

By “swelling reagent” is meant a reagent that increases the hydrodynamic size of a particle. Swelling reagents may act by increasing the volume, reducing the deformability, or changing the shape of a particle.

By “substantially larger” is meant at least 2-fold, 3-fold, 5-fold, 10-fold, 25-fold, 50-fold, or even 100-fold larger.

By “substantially smaller” is meant at least 2-fold, 3-fold, 5-fold, 10-fold, 25-fold, 50-fold, or even 100-fold smaller.

Other features and advantages will be apparent from the following description and the claims.

### BRIEF DESCRIPTION OF THE DRAWINGS

FIGS. 1A-1E are schematic depictions of an array that separated cells based on deterministic lateral displacement: (A) illustrates the lateral displacement of subsequent rows; (B) illustrates how fluid flowing through a gap is divide unequally around obstacles in subsequent rows; (C) illustrates how an analyte with a hydrodynamic size above the critical size is displaced laterally in the device; (D) illustrates an array of cylindrical obstacles; and (E) illustrates an array of elliptical obstacles.

FIG. 2 is a schematic description illustrating the unequal division of the flux through a gap around obstacles in subsequent rows.

FIG. 3 is a schematic depiction of how the critical size depends on the flow profile, which is parabolic in this example.

FIG. 4 is an illustration of how shape affects the movement of analytes through a device.

FIG. 5 is an illustration of how deformability affects the movement of analytes through a device.

FIG. 6 is a schematic depiction of deterministic lateral displacement. Analytes having a hydrodynamic size above the critical size move to the edge of the array, while analytes having a hydrodynamic size below the critical size pass through the device without lateral displacement.

FIG. 7 is a schematic depiction of a three stage deterministic device.

FIG. 8 is a schematic depiction of the maximum size and cut-off size for the device of FIG. 7.

FIG. 9 is a schematic depiction of a bypass channel.

FIG. 10 is a schematic depiction of a bypass channel.

FIG. 11 is a schematic depiction of a three stage deterministic device having a common bypass channel.

FIG. 12 is a schematic depiction of a three stage, duplex deterministic device having a common bypass channel.

FIG. 13 is a schematic depiction of a three stage deterministic device having a common bypass channel, where the flow through the device is substantially constant.

FIG. 14 is a schematic depiction of a three stage, duplex deterministic device having a common bypass channel, where the flow through the device is substantially constant.

FIG. 15 is a schematic depiction of a three stage deterministic device having a common bypass channel, where the fluidic resistance in the bypass channel and the adjacent stage are substantially constant.

FIG. 16 is a schematic depiction of a three stage, duplex deterministic device having a common bypass channel, where the fluidic resistance in the bypass channel and the adjacent stage are substantially constant.

FIG. 17 is a schematic depiction of a three stage deterministic device having two, separate bypass channels.

FIG. 18 is a schematic depiction of a three stage deterministic device having two, separate bypass channels, which are in arbitrary configuration.

FIG. 19 is a schematic depiction of a three stage, duplex deterministic device having three, separate bypass channels.

FIG. 20 is a schematic depiction of a three stage deterministic device having two, separate bypass channels, wherein the flow through each stage is substantially constant.

FIG. 21 is a schematic depiction of a three stage, duplex deterministic device having three, separate bypass channels, wherein the flow through each stage is substantially constant.

FIG. 22 is a schematic depiction of a flow-extracting boundary.

FIG. 23 is a schematic depiction of a flow-feeding boundary.

FIG. 24 is a schematic depiction of a flow-feeding boundary, including a bypass channel.

FIG. 25 is a schematic depiction of two flow-feeding boundaries flanking a central bypass channel.

FIG. 26 is a schematic depiction of a device having four channels that act as on-chip flow resistors.

FIGS. 27 and 28 are schematic depictions of the effect of on-chip resistors on the relative width of two fluids flowing in a device.

FIG. 29 is a schematic depiction of a duplex device having a common inlet for the two outer regions.

FIG. 30A is a schematic depiction of a multiple arrays on a device. FIG. 30B is a schematic depiction of multiple arrays with common inlets and product outlets on a device.

FIG. 31 is a schematic depiction of a multi-stage device with a small footprint.

FIG. 32 is a schematic depiction of blood passing through a device.

FIG. 33 is a graph illustrating the hydrodynamic size distribution of blood cells.

FIGS. 34A-34D are schematic depictions of moving an analyte from a sample to a buffer in a single stage (A), three stage (B), duplex (C), or three stage duplex (D) deterministic device.

FIG. 35A is a schematic depiction of a two stage deterministic device employed to move a particle from blood to a buffer to produce three products. FIG. 35B is a schematic graph of the maximum size and cut off size of the two stages. FIG. 35C is a schematic graph of the composition of the three products.

FIG. 36 is a schematic depiction of a two stage deterministic device for alteration, where each stage has a bypass channel.

FIG. 37 is a schematic depiction of the use of fluidic channels to connect two stages in a device.

FIG. 38 is a schematic depiction of the use of fluidic channels to connect two stages in a device, wherein the two stages are configured as a small footprint array.

FIG. 39A is a schematic depiction of a two stage deterministic device having a bypass channel that accepts output from both stages. FIG. 39B is a schematic graph of the size range of product achievable with this device.

FIG. 40 is a schematic depiction of a two stage deterministic device for alteration having bypass channels that flank each stage and empty into the same outlet.

FIG. 41 is a schematic depiction of a deterministic device for the sequential movement and alteration of particles.

FIG. 42A is a photograph of a deterministic device that may be incorporated into a device of the invention. FIGS. 42B-42E are depictions the mask used to fabricate a device that may be incorporated into the invention. FIG. 42F is a series of photographs of the device containing blood and buffer.

FIGS. 43A-43F are typical histograms generated by the hematology analyzer from a blood sample and the waste (buffer, plasma, red blood cells, and platelets) and product (buffer and nucleated cells) fractions generated by the device of FIG. 42.

FIGS. 44A-44D are depictions the mask used to fabricate a deterministic device that may be incorporated into a device of the invention.

FIGS. 45A-45D are depictions the mask used to fabricate a deterministic device that may be incorporated a device of into the invention.

FIG. 46A is a micrograph of a sample enriched in fetal red blood cells. FIG. 46B is a micrograph of maternal red blood cell waste.

FIG. 47 is a series of micrographs showing the positive identification of male fetal cells (Blue=nucleus, Red=X chromosome, Green=Y chromosome).

FIG. 48 is a series of micrographs showing the positive identification of sex and trisomy 21.

FIGS. 49A-49D are depictions the mask used to fabricate a deterministic device that may be incorporated into a device of the invention.

FIGS. 50A-50G are electron micrographs of the device of FIG. 49.

FIGS. 51A-51D are depictions the mask used to fabricate a deterministic device that may be incorporated into a device of the invention.

FIGS. 52A-52F are electron micrographs of the device of FIG. 51.

FIGS. 53A-53F are electron micrographs of the device of FIG. 45.

FIGS. 54A-54D are depictions the mask used to fabricate a deterministic device that may be incorporated a device of into the invention.

FIGS. 55A-55S are electron micrographs of the device of FIG. 54.

FIGS. 56A-56C are electron micrographs of the device of FIG. 44.

FIG. 57A is a schematic illustration of a deterministic device that may be incorporated into a device of the invention and its operation. FIG. 57B is an illustration of the device of FIG. 57A and a further-schematized representation of this device.

FIGS. 58A and 58B are illustrations of two distinct configurations for joining two deterministic devices together. In FIG. 58A, a cascade configuration is shown, in which outlet 1 of one device is joined to a sample inlet of a second device. In FIG. 58B, a bandpass configuration is shown, in which outlet 2 of one device is joined to a sample inlet of a second device.

FIG. 59 is an illustration of an enhanced method of size separation in which target cells are labeled with immunoaffinity beads.

FIG. 60 is an illustration of a method for performing size fractionation and for separating free labeling reagents, e.g., antibodies, from bound labeling reagents by using a device that may be incorporated into the invention.

FIG. 61 is an illustration of a method shown in FIG. 60. In this case, non-target cells may copurify with target cells, but these non-target cells do not interfere with quantification of target cells.

FIG. 62 is an illustration of a method for separating large cells from a mixture and producing a concentrated sample of these cells.

FIG. 63 is an illustration of a method for lysing cells inside a device of the invention and separating whole cells from organelles and other cellular components.

FIG. 64 is an illustration of two devices arrayed in a cascade configuration and used for performing size fractionation and for separating free labeling reagent from bound labeling reagents by using a device of the invention.

FIG. 65 is an illustration of two devices arrayed in a cascade configuration and used for performing size fractionation and for separating free labeling reagent from bound labeling reagents by using a device of the invention. In this figure, phage is utilized for binding and detection rather than antibodies.

FIG. 66 is an illustration of two devices arrayed in a bandpass configuration.

FIG. 67 is a graph of cell count versus hydrodynamic cell diameter for a microfluidic separation of normal whole blood.

FIG. 68 is a set of histograms from input, product, and waste samples generated with a Coulter “A^{C}-T diff” clinical blood analyzer. The x-axis depicts cell volume in femtomoles.

FIG. 69 is a pair of representative micrographs from product and waste streams of fetal blood processed with a cell enrichment module, showing clear separation of nucleated cells and red blood cells.

FIG. 70 is a pair of images showing cells fixed on a cell enrichment module with paraformaldehyde and observed by fluorescence microscopy. Target cells are bound to the obstacles and floor of the capture module.

FIG. 71A is a graph of cell count versus hydrodynamic cell diameter for a microfluidic separation of normal whole blood. FIG. 71B is a graph of cell count versus hydrodynamic cell diameter for a microfluidic separation of whole blood including a population of circulating tumor cells. FIG. 71C is the graph of FIG. 71B, additionally showing a size cutoff that excludes most native blood cells. FIG. 71D is the graph of FIG. 71C, additionally showing that the population of cells larger than the size cutoff may include endothelial cell, endometrial cells, or trophoblasts indicative of a disease state.

FIG. 72 is a schematic illustration of a method that features isolating and counting large cells within a cellular sample, wherein the count is indicative of a patient's disease state, and subsequently further analyzing the large cell subpopulation.

FIG. 73A is a design for a preferred deterministic device that may be incorporated into the invention. FIG. 73B is a table of design parameters corresponding to FIG. 73A.

FIG. 74 is a cross-sectional view of a magnetic separation device useful in a device of the invention and associated process flow for cell isolation followed by release for off-line analysis according to the present invention.

FIG. 75 is a schematic of the fabrication and functionalization of a magnetic separation device. The magnetized posts enable post-packaging modification of the device.

FIG. 76 is a schematic of an application of a magnetic separation device to capture and release CD71+ cells from a complex mixture, such as blood, using monoclonal antibodies to the transferrin (CD71) receptor.

FIG. 77 is a schematic representation of an application of a magnetic separation device to capture and release CD71+ cells from a complex mixture, such as blood, using holotransferrin. Holotransferrin is rich in iron content, commercially available, and has higher affinity constants and specificity of interaction with the CD71 receptor than its counterpart monoclonal antibody.

FIG. 78 is a schematic representation of a high-gradient magnet. The magnet is designed to generate 1.2 Tesla and ˜3 Tesla/mm.

FIG. 79A is a schematic depiction of a capillary disposed adjacent the magnet of FIG. 78. FIG. 79B is a graph showing the field strength of the magnet as a function of position in the capillary. FIG. 79C is a picture of red blood cells concentrated into discrete regions after 10 minutes in the magnetic field.

FIG. 80 is a picture of a pellet of nucleated red blood cells (positive fraction) and a pellet of white blood cells (negative fraction) prepared from male cord blood. Nucleated cells are first extracted from the blood using a deterministic lateral separation device, and treated with sodium nitrite at 50M for 10 min. The nucleated cells are then passed through a magnetic column where nucleated red blood cells are retained. In the column, the magnetic field strength is about 1 Tesla, the magnetic field gradient is about 3000 Tesla/m, and the flow velocity is about 0.4 mm/sec. White blood cells are rinsed out of the column using Dulbecco PBS buffer with 1% BSA and 2 mM EDTA, and collected as the negative fraction. The nucleated red blood cells are eluted from the column using the same buffer at a flow velocity of 4 mm/s and collected as the positive fraction.

FIG. 81 is a series of fluorescence images of nucleated red blood cells isolated from maternal blood using the method described in FIG. 80. The cells are stained using fluorescence in situ hybridization (FISH). The X chromosome is identified with an aqua labeled probe for the alpha satellite region, while the Y chromosome is identified with red and green stains for the alpha satellite and satellite III regions, respectively. The nuclei are counterstained with DAPI (blue).

FIG. 82 shows nucleated red blood cells in different maturation stages isolated from maternal blood using the method described in FIG. 80. The cells are stained with Wright-Geimsa stain.

FIGS. 83A and 83B show micrographs of results of enrichment employing anti-CD71 antibodies (A) and the method described in FIG. 80 (B). The sample in A contained >200,000 nucleated cells from 1 mL of blood, while the sample in B contained about 100-500 nucleated cells per mL of blood. The purity of nucleated red blood cells obtained by the method described in FIG. 80 is about 1000 times better than antibody-based enrichment methods.

FIG. 84 shows schematic depictions of three methods of the invention.

FIG. 85 shows a schematic depiction of an integrated device of the invention.

Figures are not necessarily to scale.

### DETAILED DESCRIPTION OF THE INVENTION

The invention provides analytical devices and methods useful for enriching analytes in a sample. In general, enrichment occurs through the interaction of analytes, or other components of a sample, with a magnetic field. Analytes may be enriched based on an intrinsic magnetic property (e.g., iron containing proteins), an extrinsic magnetic property (e.g., magnetic beads bound to an analyte), or lack of any intrinsic or extrinsic magnetic properties. Enrichment may occur based on existing magnetic properties of components of a sample, or based on reaction with a reagent capable of altering (e.g., inducing or adding) a magnetic property. The methods and devices of the present invention may be used to produce enriched samples of analytes, such as red blood cells (e.g., fetal red blood cells from maternal blood).

Magnetic Separation

The intrinsic, e.g., altered as in the methods described herein, or extrinsic magnetic properties, e.g., as provided by a magnetic bead, of an analyte may be used to effect an isolation, enrichment, or depletion of the analyte relative to other components of a sample. The isolation, enrichment, or depletion may include positive selection, i.e., a desired analyte is attracted to a magnetic field, or it may employ negative selection, i.e., a desired analyte is not attracted to the magnetic field, e.g., repulsed or unaffected. In either case, the population of analytes containing the desired analytes may be collected for analysis or further processing.

The device used to perform the magnetic separation may be any device that can produce a magnetic field. In one embodiment, a MACS column (e.g., from Miltenyi Biotec) is used to effect enrichment of a magnetically responsive analyte. If the analyte is magnetically responsive, e.g., by reaction with a reagent as described herein, it will be attracted to the MACS column under a magnetic field, thereby permitting enrichment of the desired analyte relative to other constituents of the sample. In another embodiment, enrichment may be achieved using a device, typically microfluidic, that contains a plurality of magnetic obstacles. If an analyte in the sample is magnetically responsive (e.g., through reaction with a reagent that alters an intrinsic magnetic property of the analyte or by binding of a magnetically responsive particle to the analyte), the analyte may bind to the obstacles, thereby permitting enrichment of the bound analyte. Alternatively, negative selection may be employed. In this example, the desired analyte may be, or may be rendered, magnetically unresponsive, or an undesired analyte may be, or may be rendered, magnetically responsive or bound to a magnetically responsive particle. In this case, an undesired analyte or analytes will be retained in the magnetic device whereas the desired analyte will not, thus enriching the sample in the desired analyte.

In another embodiment, the sample is treated with a reagent that includes magnetic particles prior to application of a magnetic field. As described herein, the magnetic particles may be coated with appropriate capture moieties such as antibodies to which an analyte can bind. Application of a magnetic field to the treated sample will selectively attract an analyte bound to magnetic particles.

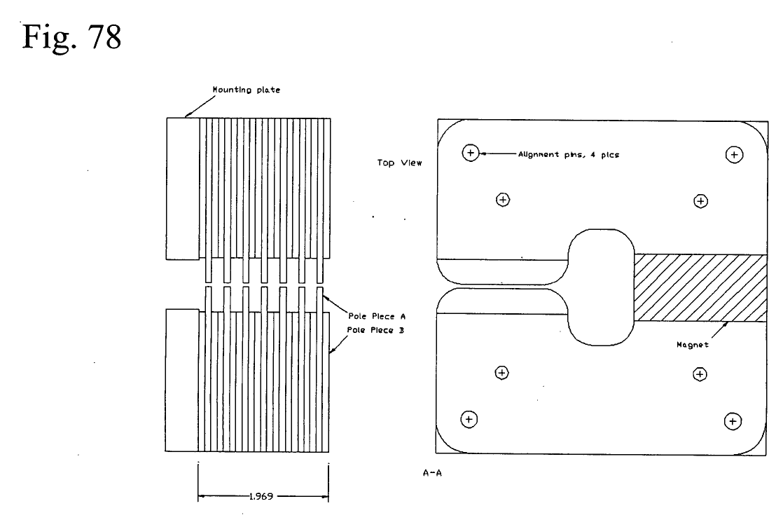

Channels or other regions of the device may, or may not be, magnetically responsive. In one embodiment, a channel through which analytes pass is coupled to a magnet capable of producing an appropriate magnetic field within the channel. An exemplary magnet is shown in FIG. 78. Alternatively, a channel in a device contains magnetically responsive regions, which typically alter an applied magnetic field. Typically, the magnetic field strength is 0.05 to 5.0 Tesla, e.g., about 0.5 Tesla, and the magnetically responsive regions generate field gradients of 100 to 1,000,000 Tesla/m, e.g., about 10⁴ Tesla/m.

Magnetic regions of the device can be fabricated with either hard or soft magnetic materials, such as, but not limited to, iron, steel, nickel, cobalt, rare earth materials, neodymium-iron-boron, ferrous-chromium-cobalt, nickel-ferrous, cobalt-platinum, and strontium ferrite. Portions of the device may be fabricated directly out of magnetic materials, or the magnetic materials may be applied to another material. The use of hard magnetic materials can simplify the design of a device because they are capable of generating a magnetic field without other actuation. Soft magnetic materials, however, enable release and downstream processing of bound analytes simply by demagnetizing the material. Depending on the magnetic material, the application process can include cathodic sputtering, sintering, electrolytic deposition, or thin-film coating of composites of polymer binder-magnetic powder. A preferred embodiment is a thin film coating of micromachined obstacles (e.g., silicon posts) by spin casting with a polymer composite, such as polyimide-strontium ferrite (the polyimide serves as the binder, and the strontium ferrite as the magnetic filler). After coating, the polymer magnetic coating is cured to achieve stable mechanical properties. After curing, the device is briefly exposed to an external induction field, which governs the preferred direction of permanent magnetism in the device. The magnetic flux density and intrinsic coercivity of the magnetic fields from the obstacles can be controlled by the % volume of the magnetic filler.

In another embodiment, an electrically conductive material is micropatterned on the outer surface of an enclosed microfluidic device. The pattern may consist of a single, electrical circuit with a spatial periodicity of approximately 100 microns. By controlling the layout of this electrical circuit and the magnitude of the electrical current that passes through the circuit, one can develop periodic regions of higher and lower magnetic strength within the enclosed microfluidic device.

In yet another embodiment, the magnetically responsive region includes packed beads of iron with non-sticking plastic or Teflon coating.

For any of the above embodiments, any source of a magnetic field may be employed in the invention and may include hard magnets, soft magnets, electromagnets, superconductor magnets, or a combination thereof. In one embodiment, a spatially nonuniform permanent magnet or electromagnet may be used to create organized and in some cases periodic arrays of magnetic particles within an otherwise untextured microfluidic channel (Deng et al. Applied Physics Letters, 78, 1775 (2001)). Alternatively, a nonuniform magnetic field may be employed that does not have a regular periodicity. An electromagnet may be employed to create a non-uniform magnetic field in a device. The non-uniform filed creates regions of higher and lower magnetic field strength, which, in turn, will attract magnetic particles in a periodic arrangement within the device. Other external magnetic fields may be employed to create magnetic regions to which magnetic particles attach. A hard magnetic material may also be used in the fabrication of the device, thereby obviating the need for electromagnets or external magnetic fields. In one embodiment, the device contains a plurality of channels having magnetic regions, e.g., to increase volumetric throughput. Further, these channels may be stacked vertically.

In the above embodiments, an analyte bound to a magnet can be released from defined locations within the channel, e.g., by increasing the overall flow rate of the fluid flowing through the device, decreasing the magnetic field, or through some combination of the two.

The magnetic field can be adjusted to influence supra and paramagnetic particles with magnetic mass susceptibility, e.g., ranging from 0.1-200×10 m³/kg. The paramagnetic particles of use can be classified based on size: particulates (1-5 μm in the size of a cell diameter); colloidal (on the order of 100 nm); and molecular (on the order of 2-10 nm). The fundamental force acting on a paramagnetic entity is:

$${F}_{b} = \frac{1}{2 {μ}_{o}} Δ χ {V}_{G} ∇ {B}^{2}$$

where F_{b} is the magnetic force acting on the paramagnetic entity of volume V_{b}, Δ_{χ} is the difference in magnetic susceptibility between the magnetic particle, _{χ}b, and the surrounding medium, _{χf, μ}ₒ is the magnetic permeability of free space, B is the external magnetic field, and ∇ is the gradient operator. The magnetic field can be controlled and regulated to enable attraction and retention of a wide spectrum of particulate, colloidal, and molecular paramagnetic entities.

Reagents Capable of Altering a Magnetic Property

In certain embodiments, analytes, or other components of a sample, are reacted with a reagent capable of altering a magnetic property, either intrinsic or extrinsic, of the analyte or other component. The exact nature of the reagent will depend on the nature of the analyte and whether the reagent will modify an intrinsic or extrinsic magnetic property. Exemplary reagents include agents that oxidize or reduce transition metals, magnetic beads capable of binding to an analyte, or reagents that are capable of chelating or otherwise binding iron (e.g., as described in U.S. Pat. No. 4,508,625), or other magnetic materials or particles. Specific reagents include chemicals, e.g. sodium nitrite, gases, e.g. nitrogen, oxygen, carbon dioxide, carbon monoxide, and mixtures thereof. For example, a reagent may act to paragmagnetize or diamagnetize an analyte. A reagent may also act to deoxygenate an analyte, e.g., myoglobin or hemoglobin. The reagent may act to alter the magnetic properties of an analyte to enable, decrease, or increase its attraction to a magnetic field, to enable, decrease, or increase its repulsion to a magnetic field, or to eliminate a magnetic property such that the analyte is unaffected by a magnetic field. The reagent may also alter the magnetic properties of fluids in which the analytes are dissolved, suspended, or otherwise carried, or magnetic properties of the cytosol of a cell. A reagent may also alter the rheology of an analyte.

In certain embodiments, magnetic particles are bound to analytes to impart extrinsic magnetic responsiveness. For these embodiments, any particle that responds to a magnetic field may be employed in the devices and methods of the invention. Desirable particles are those that have surface chemistry that can be chemically or physically modified, e.g., by chemical reaction, physical adsorption, entanglement, or electrostatic interaction. Magnetic particles of the present invention can come in any size and/or shape. In some embodiments, a magnetic particle has a diameter of less than 500 nm, 400 nm, 300 nm, 200 nm, 100 nm, 90 nm, 80 nm, 70 nm, 60 nm or 50 nm. In some embodiments, a magnetic particle has a diameter that is between 10-1000 nm, 20-800 nm, 30-600 nm, 40-400 nm, or 50-200 nm. In some embodiments, a magnetic particle has a diameter of more than 10 nm, 50 nm, 100 nm, 200 nm, 500 nm, 1000 nm, or 5000 nm. The magnetic particles can be dry or in liquid form. Mixing of a fluid sample with a second liquid medium containing magnetic particles can occur by any means known in the art.

Capture moieties can be bound to magnetic particles by any means known in the art. Examples include chemical reaction, physical adsorption, entanglement, or electrostatic interaction. The capture moiety bound to a magnetic particle will depend on the nature of the analyte targeted. Examples of capture moieties include, without limitation, proteins (such as antibodies, avidin, and cell-surface receptors), charged or uncharged polymers (such as polypeptides, nucleic acids, and synthetic polymers), hydrophobic or hydrophilic polymers, small molecules (such as biotin, receptor ligands, and chelating agents), and ions. Such capture moieties can be used to bind cells specifically (e.g., bacterial, pathogenic, fetal cells, fetal blood cells, cancer cells, epithelial cells, endothelial cells, and blood cells), organelles (e.g., nuclei), viruses, peptides, protein, polymers, nucleic acids, supramolecular complexes, other biological molecules (e.g., organic or inorganic molecules), small molecules, ions, or combinations or fragments thereof. Specific examples of capture moieties include anti-CD71, anti-CD36, anti-GPA, anti-EpCAM, anti-E-cadherin, anti-Muc-1, and holo-transferrin. In another embodiment, the capture moiety is fetal cell (e.g., fetal red blood cell), cancer cell, or epithelial cell specific.

A sample may also be combined with a reagent that alters an intrinsic magnetic property of an analyte. The altered analyte may be rendered more or less magnetically responsive or may be rendered magnetically unresponsive by the reagent as compared to the unaltered analyte. In one example, a sample (e.g., a maternal blood sample that has, for example, been depleted of maternal red blood cells) containing fetal red blood cells (fRBCs) is treated with sodium nitrite, thereby causing oxidation of fetal hemoglobin contained within the fRBCs. This oxidation alters the magnetic responsiveness of the fetal hemoglobin relative to other components of the sample, e.g., maternal white blood cells, thereby allowing separation of the fRBCs. In addition, differential oxidation of fetal and maternal cells could be used to separate fetal versus maternal nucleated RBCs. Any cell containing magnetically responsive components such as iron found in hemoglobin (e.g., adult or fetal), myoglobin, or cytochromes (e.g., cytochrome C) may be modified to alter intrinsic magnetic responsiveness of an analyte such as a cell, or a component thereof (e.g., an organelle). Furthermore, cells may be contacted with reagents that induce, prevent, increase, or decrease expression of proteins or other molecules that are magnetically responsive.

Analytical Devices

The devices of the invention may be employed in connection with or include any analytical device. Examples include affinity columns, cell counters, particle sorters, e.g., fluorescent activated cell sorters and magnetic activated cell sorters, capillary electrophoresis, sample storage devices, and sample preparation devices. Microfluidic devices are of particular interest in connection with the systems described herein.

Exemplary analytical devices include devices useful for size, shape, or deformability based separation of particles, including filters, sieves, and deterministic separation devices, e.g., those described in International Publication Nos. 2004/029221 and 2004/113877, Huang et al. Science 304, 987-990 (2004), U.S. Publication No. 2004/0144651, U.S. Pat. Nos. 5,837,115 and 6,692,952, and U.S. Application Nos. 60/703,833 and 60/704,067; devices useful for affinity capture, e.g., those described in International Publication No. 2004/029221 and U.S. application Ser. No. 11/071,679; devices useful for preferential lysis of cells in a sample, e.g., those described in International Publication No. 2004/029221, U.S. Pat. No. 5,641,628, and U.S. Application No. 60/668,415; and devices useful for arraying cells, e.g., those described in International Publication No. 2004/029221, U.S. Pat. No. 6,692,952, and U.S. application Ser. Nos. 10/778,831 and 11/146,581. Two or more devices, either the same or different devices, may be combined in series or integrated into a single device, e.g., as described in International Publication No. 2004/029221.

In particular embodiments, the analytical device may be used to enrich various analytes in a sample, e.g., for collection or further analysis. Rare cells or components thereof can be retained in the device, or otherwise enriched, compared to other cells as described, e.g., in International Publication No. 2004/029221. Exemplary rare cells include, depending on the sample, fetal cells (e.g., fetal red blood cells); stem cells (e.g., undifferentiated); cancer cells; immune system cells (host or graft); epithelial cells; connective tissue cells; bacteria; fungi; viruses; and pathogens (e.g., bacterial or protozoa). Such rare cells may be isolated from samples including bodily fluids, e.g., blood, or environmental sources, e.g., water or air samples. Fetal red blood cells may be enriched from maternal peripheral blood, e.g., for the purpose of determining sex and identifying aneuploidies or genetic characteristics, e.g., mutations, in the developing fetus. Cancer cells may also be enriched from peripheral blood for the purpose of diagnosis and monitoring therapeutic progress. Bodily fluids or environmental samples may also be screened for pathogens, e.g., for coliform bacteria, blood borne illnesses such as sepsis, or bacterial or viral meningitis. Rare cells also include cells from one organism present in another organism, e.g., cells from a transplanted organ. Analytes retained or otherwise enriched in the device may, for example, be labeled, e.g., with fluorescent or radioactive probes, subjected to chemical or genetic analysis (such as fluorescent in situ hybridization), if biological, cultured, or otherwise observed or probed.

Analytical devices may or may not include microfluidic channels, i.e., may or may not be microfluidic devices. The dimensions of the channels of the device into which analytes are introduced may depend on the size or type of analytes employed. Preferably, a channel in an analytical device has at least one dimension (e.g., height, width, length, or radius) of no greater than 10, 9.5, 9, 8.5, 8, 7.5, 7, 6.5, 6, 5.5, 5, 4.5, 4, 3.5, 3, 2.5, 2, 1.5, or 1 mm. Microfluidic devices employed in the systems and methods described herein preferably have at least one dimension of less than 1, 0.9, 0.8, 0.7, 0.6, 0.5, 0.4, 0.3, 0.2, 0.1, or even 0.05 mm. The preferred dimensions of an analytical device can be determined by one skilled in the art based on the desired application.

An analytical device (e.g., a deterministic device) may be coupled to, or otherwise include, a reservoir containing a reagent (e.g., magnetic particles having a binding moiety or sodium nitrite) capable of altering a magnetic property of an analyte (e.g., a cell such as a red blood cell). The reservoir may include a channel, e.g., a microfluidic channel, a tube, or any other container capable of receiving the analyte and contacting it with the reagent. The reservoir may be separable from the analytical device or may be integrated with it. Mixing of the reagent with the analyte may occur by any means including diffusion, mechanical mixing, chaotic mixing, convection, or turbulent flow. The reagent may be stored dry in the reservoir and liquefied upon introduction of a sample or stored in solution and mixed with the sample. In another embodiment, the reagent is added continuously or in a discrete bolus to the reservoir concomitant with the delivery of the sample.

The reservoir may also include structures that allow for the separation of the altered analyte from the unreacted reagent or byproducts of reaction of the reagent with the analyte. For example, deterministic separation may be employed for this purpose as described herein. Alternatively, filters, rinses, or other means may be employed. Such a structure may or may not be included as part of the reservoir or analytical device.

In one embodiment, the reservoir includes a channel having magnetic regions in a textured surface with which an analyte passing through the channel can come into contact, e.g., through attaching magnetic particle to regions in a channel. Through the appropriate choice of parameters, e.g., magnetic particle size and shape, relative to the dimensions of the channel, a texture that enhances interactions between an analyte and the bound magnetic particles can be provided. The magnetic particles may be coated with appropriate capture moieties such as antibodies (e.g., anti-CD71, anti-CD36, anti-CD45, anti-GPA, anti-antigen i, anti-CD34, anti-fetal hemoglobin, anti-EpCAM, anti-E-cadherin, or anti-Muc-1) that can bind to an analyte through affinity mechanisms. The magnetic particles can be disposed uniformly throughout a device or in spatially resolved regions. In addition, magnetic particles may be used to create structure within the device. For example, two magnetic regions on opposite sides of a channel can be used to attract magnetic particles to form a “bridge” linking the two regions. The magnetic particles can be magnetically attached to hard magnetic regions of the channel or to soft magnetic regions that are actuated to produce a magnetic field.

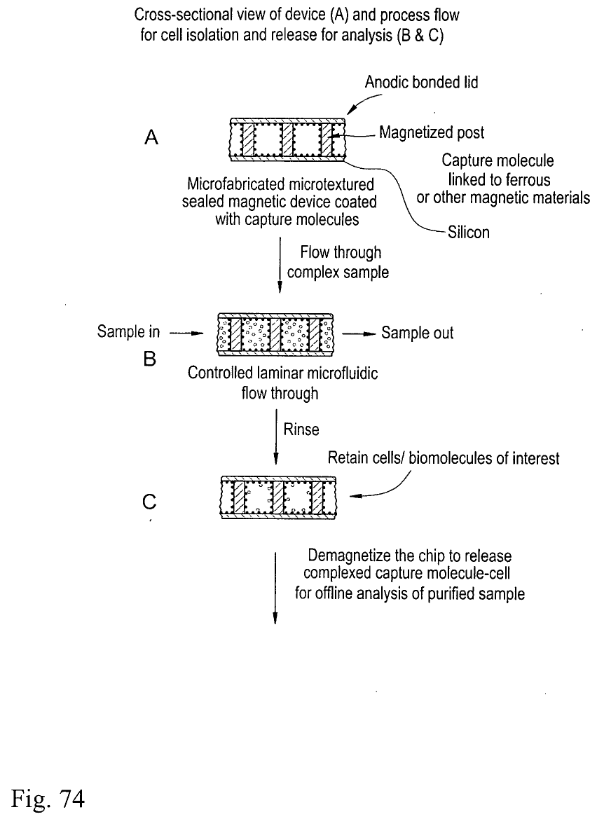

An example of a reservoir is shown in FIG. 74, which illustrates a reservoir geometry and functional process flow to isolate and then release target analytes, e.g., cells or molecules, from a complex mixture. As shown, the reservoir contains obstacles that extend from one channel surface toward the opposing channel surface. The obstacles may or may not extend the entire distance across the channel. In the present example, the obstacles are magnetic (e.g., contain hard or soft magnetic materials or are locations of high magnetic field in a non-uniform field) and attract and retain magnetic particles, which may be coated with capture moieties or may be cells attracted to a magnetic field. The geometry of the reservoir, the distribution, shape, size of the obstacles and the flow parameters can be altered to optimize the efficiency of the enrichment of an analyte of interest, for example, by attracting an analyte bound to a magnetic particle with a capture moiety (e.g., as described in International Publication No. 2004/029221). In one specific example, an anodic lidded silicon wafer with microtextured magnetic obstacles of varying shapes (cylindrical, rectangular, trapezoidal, or pleomorphic) and size (10-999 microns) are arranged uniquely (spacing and density varied across equilateral triangular, diagonal, and random array distribution) to maximize the collision frequency of analytes, altered or not, with the obstacles within the confines of a continuous perfusion flow stream. The exact geometry of the magnetic obstacles and the distribution of obstacles may depend on the type of analytes being isolated, enriched, or purified.

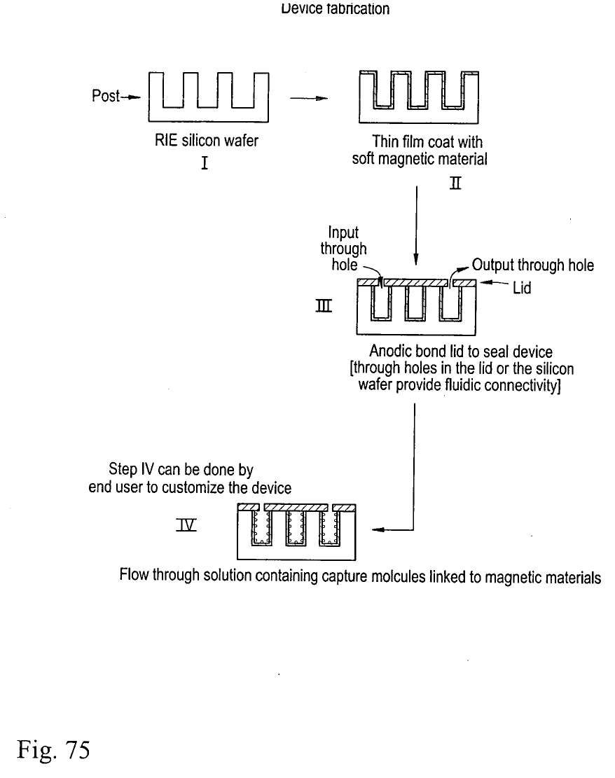

FIG. 75 illustrates an example of reservoir fabrication and functionalization. The magnetized obstacles enable post-packaging modification of the reservoir. This is a very significant improvement over existing art. The incompatibility of semiconductor processing parameters (high heat, or solvent sealers to bond the lid) with capture moieties (sensitive to temperature and inorganic and organic solvents) makes this device universal and compatible for functionalization with all capture moieties. Retention of the capture moieties on the obstacles (e.g., posts) by use of magnetic fields, is an added advantage over prior art that uses complex surface chemistry for immobilization. The reservoir enables the end user to easily and rapidly charge the reservoir with a capture moiety, or mixture of capture moieties, of choice thereby increasing the versatility of use. On-demand and ‘just-in-time’ one step functionalization is enabled by this reservoir, thereby circumventing issues of on-the-shelf stability of the capture moieties if they were chemically cross-linked at production. The capture moieties that can be loaded and retained on the obstacles include, but not limited to, all of the cluster of differentiation (CD) receptors on mammalian cells, synthetic and recombinant ligands for cell receptors, and any other organic, inorganic molecule, or compound of interest that can be attached to any magnetic particle.

Additional Components

Devices of the invention may also include elements, e.g., for isolation, collection, manipulation, or detection of an analyte. Such elements are known in the art. For example, a device of the invention (e.g., a device incorporating a deterministic device) may also include components for other types of separation, including affinity, electrophoretic, centrifugal, and dielectrophoretic separation. Devices of the invention may also include a component for two-dimensional imaging of the output from the device, e.g., an array of wells or a planar surface. Such an array of wells or planar surface may be imaged or observed through a microscope or other visual instrument, e.g., a camera.

Devices of the invention may also be employed in conjunction with other enrichment devices, either on the same device or in different devices. Other enrichment techniques are described, e.g., in International Publication Nos. 2004/029221 and 2004/113877, U.S. Pat. No. 6,692,952, and U.S. application Ser. Nos. 11/071,270, 11/071,679, and 60/668,415, each of which is incorporated by reference.

Deterministic Separation

In one embodiment, the invention provides a device that includes a channel that deterministically directs particles based on hydrodynamic size and a magnetic force generator, e.g., in conjunction with a reservoir containing a reagent capable of altering a magnetic property of the particle. The invention also provides a method for producing a sample enriched in a first analyte relative to a second analyte by applying the sample to a device that includes a channel that deterministically deflects particles based on hydrodynamic size, thereby producing a second sample enriched in the first analyte, combining the second sample with a reagent that alters a magnetic property of the first analyte, or relying on an existing magnetic property, and applying a magnetic field thereby separating the first analyte from the second analyte.

In one example, the channel includes one or more arrays of obstacles that allow deterministic lateral displacement of components of fluids. Such devices are described, e.g., in Huang et al. Science 304, 987-990 (2004) and U.S. Publication No. 20040144651, and U.S. Application No. 60/414,258. These devices may further employ an array of a network of gaps, wherein a fluid passing through a gap is divided unequally into subsequent gaps. In one embodiment, fluid passing through a gap is divided unequally even though the gaps are identical in dimensions. A flow carries particles to be separated through the array of gaps. The flow is aligned at a small angle (flow angle) with respect to a line-of-sight of the array. Particles having a hydrodynamic size larger than a critical size migrate along the line-of-sight in the array, whereas those having a hydrodynamic size smaller than the critical size follow the flow in a different direction. Flow in the device occurs under laminar flow conditions.

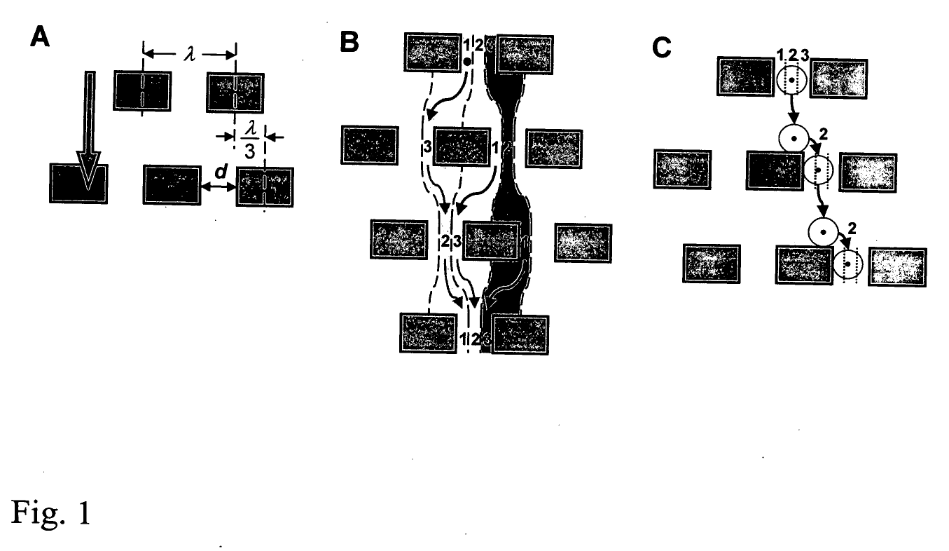

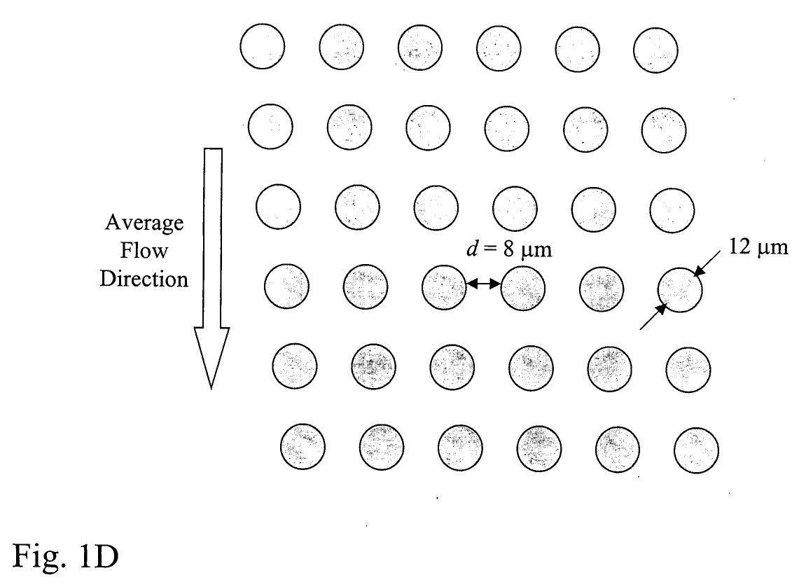

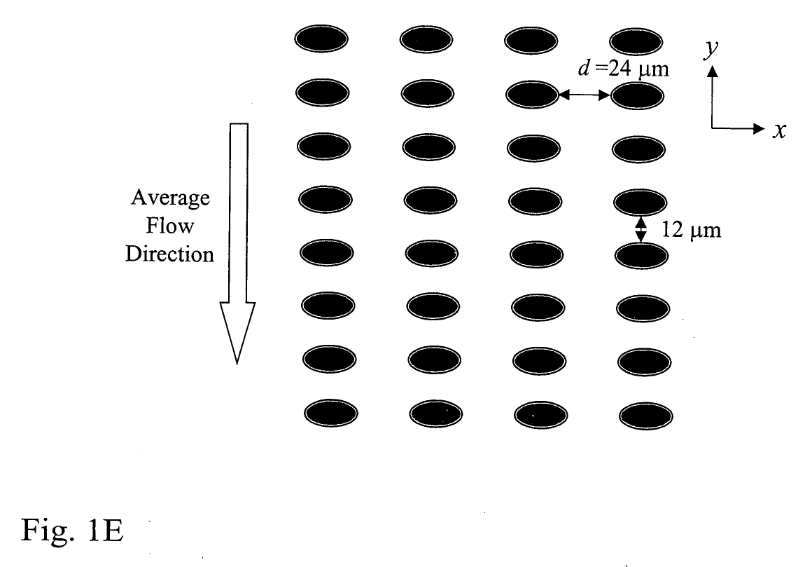

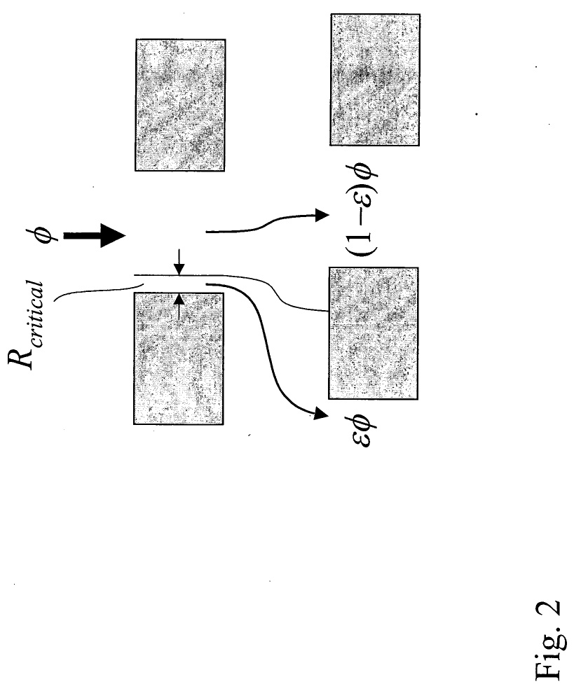

The critical size is a function of several design parameters. With reference to the obstacle array in FIG. 1, each row of obstacles is shifted horizontally with respect to the previous row by Δλ, where λ is the center-to-center distance between the obstacles (FIG. 1A). The parameter Δλ/λ (the “bifurcation ratio,” ε) determines the ratio of flow bifurcated to the left of the next obstacle. In FIG. 1, ε is 1/3, for the convenience of illustration. In general, if the flux through a gap between two obstacles is φ, the minor flux is εφ, and the major flux is (1−εφ) (FIG. 2). In this example, the flux through a gap is divided essentially into thirds (FIG. 1B). While each of the three fluxes through a gap weaves around the array of obstacles, the average direction of each flux is in the overall direction of flow. FIG. 1C illustrates the movement of an analyte sized above the critical size (e.g., 4, 5, 6, 7, 8, 9, 10, 11, 12, 13, 14, 15, 16, 17, 18, 19, 20, 21, 22, 23, 24, 25, 26, 27, 28, 29, or 30 microns) through the array. Such analytes move with the major flux, being transferred sequentially to the major flux passing through each gap.

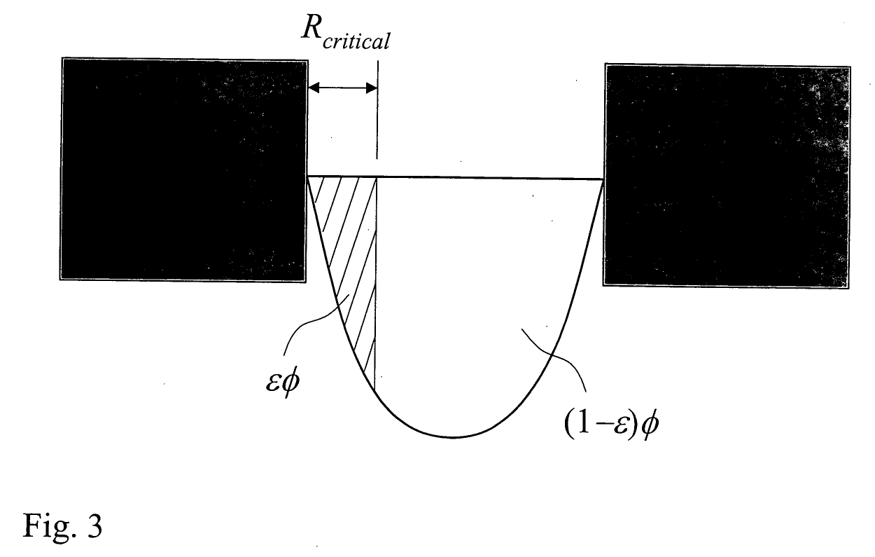

Referring to FIG. 2, the critical size is approximately 2R_{critical}, where R_{critical} is the distance between the stagnant flow line and the obstacle. If the center of mass of a particle, e.g., a cell, falls within R_{critical}, the particle would follow the major flux and move along the line-of-sight of the array. R_{critical} can be determined if the flow profile across the gap is known (FIG. 3); it is the thickness of the layer of fluids that would make up the minor flux. For a given gap size, d, R_{critical} can be tailored based on the bifurcation ratio, ε. In general, the smaller ε, the smaller R_{critical}.

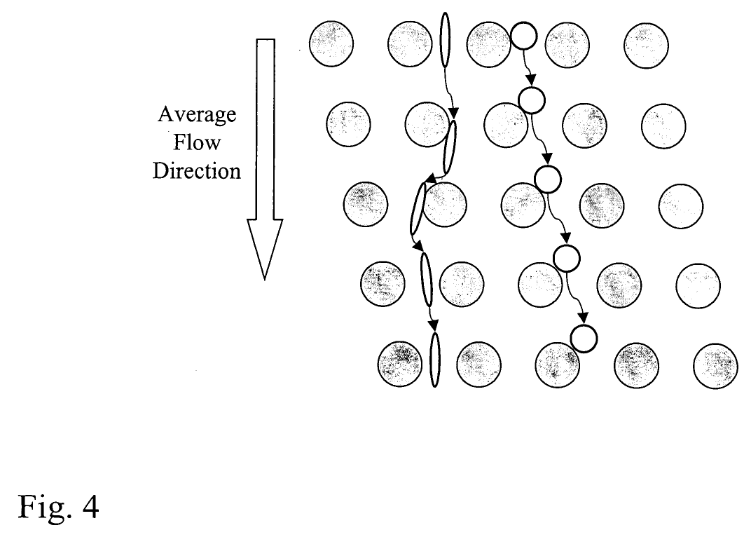

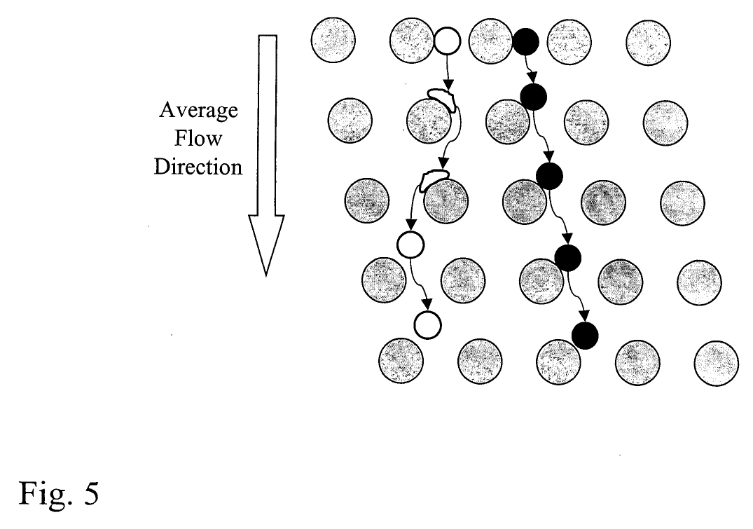

In an array for deterministic lateral displacement, particles of different shapes behave as if they have different sizes (FIG. 4). For example, lymphocytes are spheres of ˜5 μm diameter, and erythrocytes are biconcave disks of ˜7 μm diameter, and ˜1.5 μm thick. The long axis of erythrocytes (diameter) is larger than that of the lymphocytes, but the short axis (thickness) is smaller. If erythrocytes align their long axes to a flow when driven through an array of obstacles by the flow, their hydrodynamic size is effectively their thickness (˜1.5 μm), which is smaller than lymphocytes. When an erythrocyte is driven through an array of obstacles by a hydrodynamic flow, it tends to align its long axis to the flow and behave like a ˜1.5 μm-wide particle, which is effectively “smaller” than lymphocytes. The method and device may therefore separate analytes according to their shapes, although the volumes of the analytes could be the same. In addition, analytes having different deformabilities behave as if they have different sizes (FIG. 5). For example, two analytes having the same undeformed shape may be separated by deterministic lateral displacement, as one analyte may deform more readily than the other analyte when it contacts an obstacle in the array and changes shape. Thus, separation in the device may be achieved based on any parameter that affects hydrodynamic size including the physical dimensions, the shape, and the deformability of the analyte.

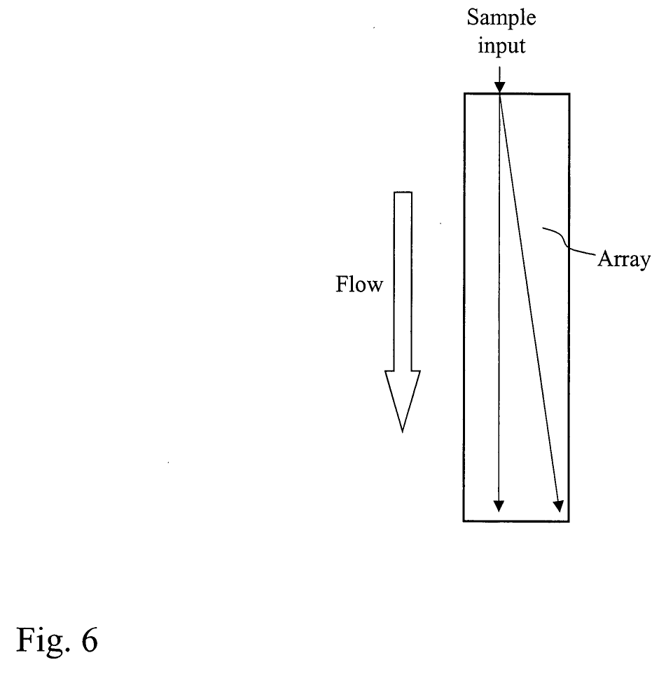

Referring to FIG. 6, feeding a mixture of analytes, e.g., cells, of different hydrodynamic sizes from the top of the array and collecting the analytes at the bottom, as shown schematically, can produce two products, an output containing analytes larger than the critical size, 2R_{critical}, and an output containing cells smaller than the critical size. Either output or both outputs may be collected, e.g., when fractionating a sample into two or more sub-samples. Analytes larger than the gap size will get trapped inside the array. Therefore, an array has a working size range. Cells have to be larger than a cut-off size (2R_{critical}) and smaller than a maximum pass-through size (array gap size) to be directed into the major flux. The “size range” of an array is defined as the ratio of maximum pass-through size to cut-off size.

Separation of Free, Unreacted Reagent from Altered Analyte

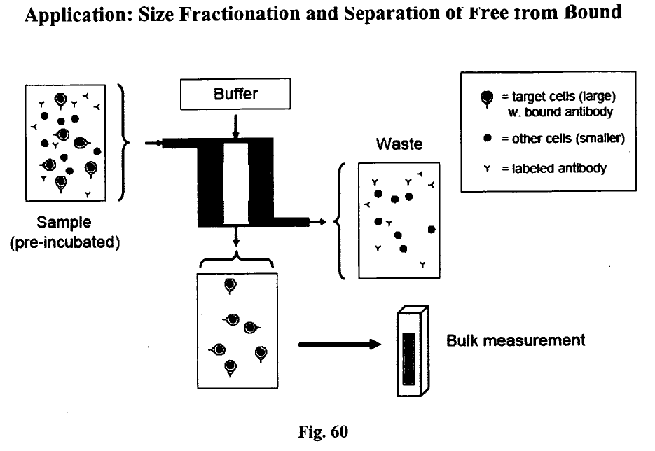

Deterministic devices may be employed in order to separate free, unreacted reagent from the altered analyte. As shown in FIG. 60, a labeling reagent such as an antibody may be pre-incubated with an analyte (e.g., a cellular sample) prior to introduction to or within the deterministic device. Desirably, the reagent specifically reacts with the analyte of interest, e.g., a cell population such as epithelial cells. Exemplary labeling reagents include antibodies, quantum dots, phage, aptamers, fluorophore-containing molecules, enzymes capable of carrying out a detectable chemical reaction, sodium nitrite, or functionalized beads. Generally, the reagent is smaller than the analyte (e.g., a cell) of interest, or the analyte of interest bound to a bead; thus, when the sample combined with the reagent is introduced to the device, unreacted reagent moves through the device undeflected, while an altered analyte (e.g., an analyte bound to the reagent) is deflected, thereby separating the unreacted reagent from the altered analyte. Advantageously, this method achieves both size separation and separation of free, unreacted reagent from the analyte. Additionally, this method of separation facilitates downstream sample analysis, if desired, without the need for a release step or a potentially destructive method of analysis, as described below.

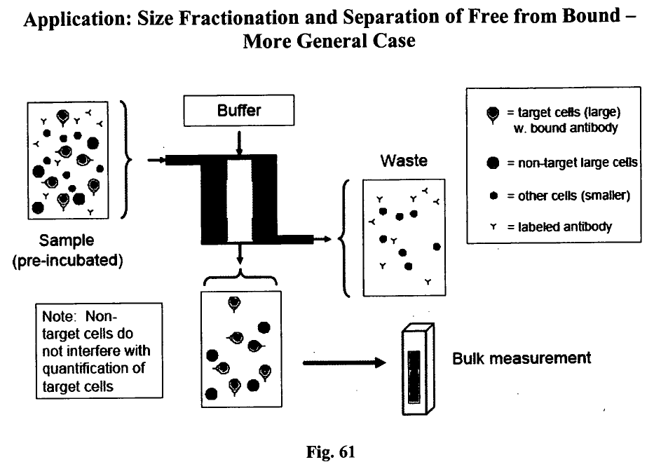

FIG. 61 shows a particular case in which the enriched, labeled sample contains a population of non-target cells that co-separate with the target cells due to similar size. The non-target cells do not interfere with downstream sample analysis that relies on detection of the bound labeling reagent, because this reagent binds selectively to the cells of interest.

Array Design

Deterministic separation may be achieved using an array of gaps and obstacles in a channel. Exemplary configurations of such arrays, bypass channels, and boundaries are described as follows.

Single-stage array. In one embodiment, a single stage contains an array of obstacles, e.g., cylindrical posts (FIG. 1D). In certain embodiments, the array has a maximum pass-through size that is several times larger than the cut-off size, e.g., when separating white blood cells from red blood cells. This result may be achieved using a combination of a large gap size d and a small bifurcation ratio ε. In preferred embodiments, the ε is at most 1/2, e.g., at most 1/3, 1/10, 1/30, 1/100, 1/300, or 1/1000. In such embodiments, the obstacle shape may affect the flow profile in the gap; however, the obstacles can be compressed in the flow direction, in order to make the array short (FIG. 1E). Single stage arrays may include bypass channels as described herein.

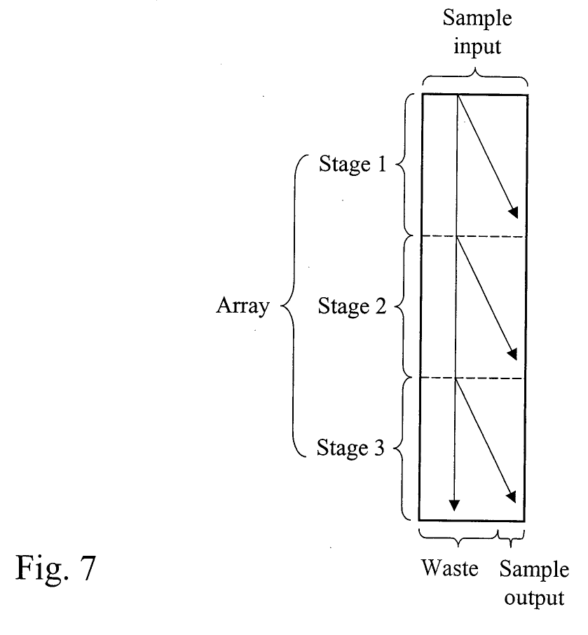

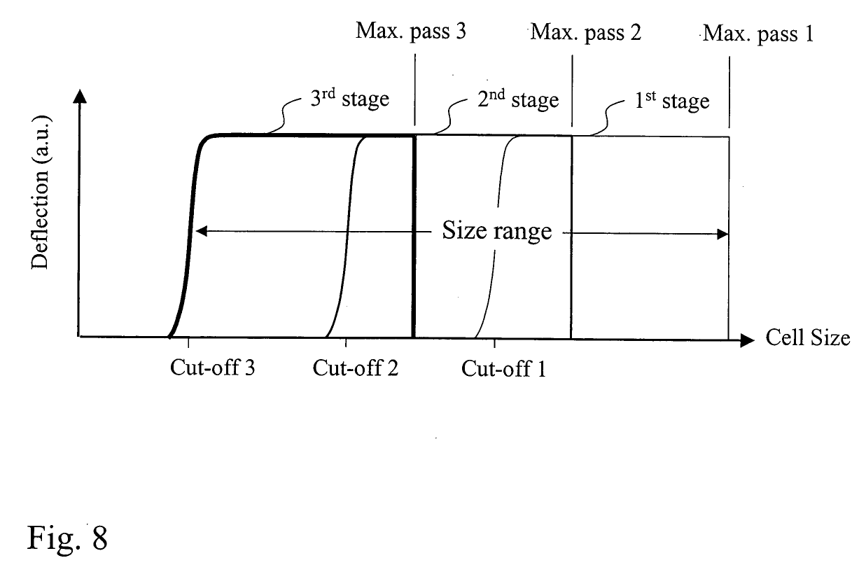

Multiple-stage arrays. In another embodiment, multiple stages are employed to separate analytes over a wide size range. An exemplary device is shown in FIG. 7. The device shown has three stages, but any number of stages may be employed, and an array can have as many stages as desired. Typically, the cut-off size in the first stage is larger than the cut-off in the second stage, and the first stage cut-off size is smaller than the maximum pass-through size of the second stage (FIG. 8). The same is true for the following stages. The first stage will deflect (and remove) analytes, e.g., that would cause clogging in the second stage, before they reach the second stage. Similarly, the second stage will deflect (and remove) analytes that would cause clogging in the third stage, before they reach the third stage.

As described, in a multiple-stage array, large analytes, e.g., cells, that could cause clogging downstream are deflected first, and these deflected analytes need to bypass the downstream stages to avoid clogging. Thus, devices of the invention may include bypass channels that remove output from an array. Although described here in terms of removing analytes above the critical size, a bypass channel may also be employed to remove output from any portion of the array.

Different Designs for Bypass Channels are as Follows.

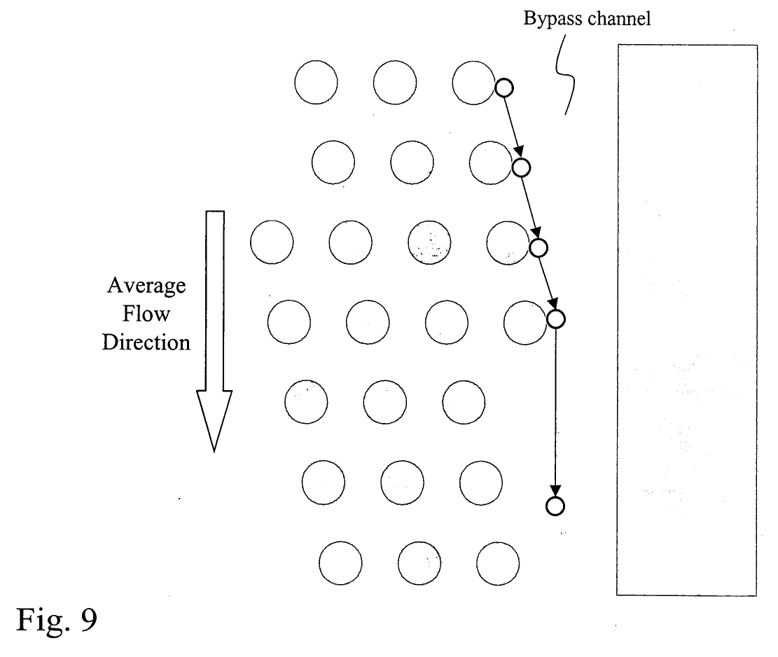

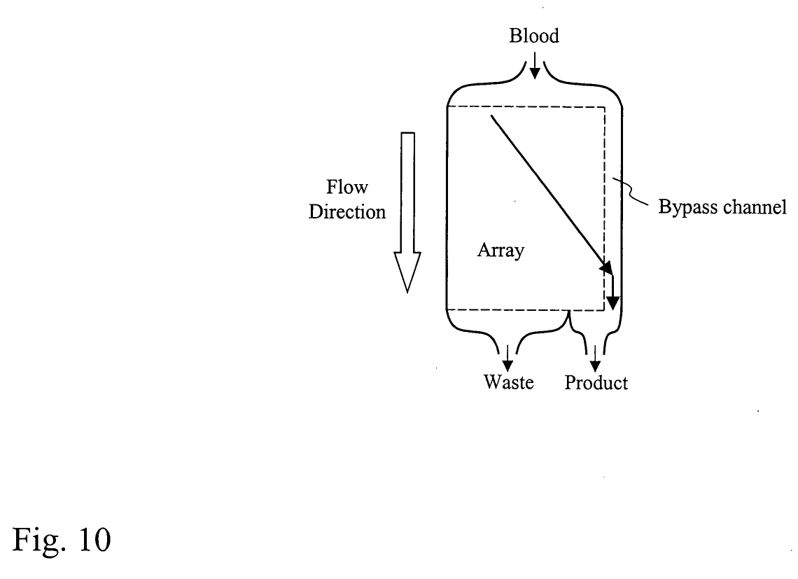

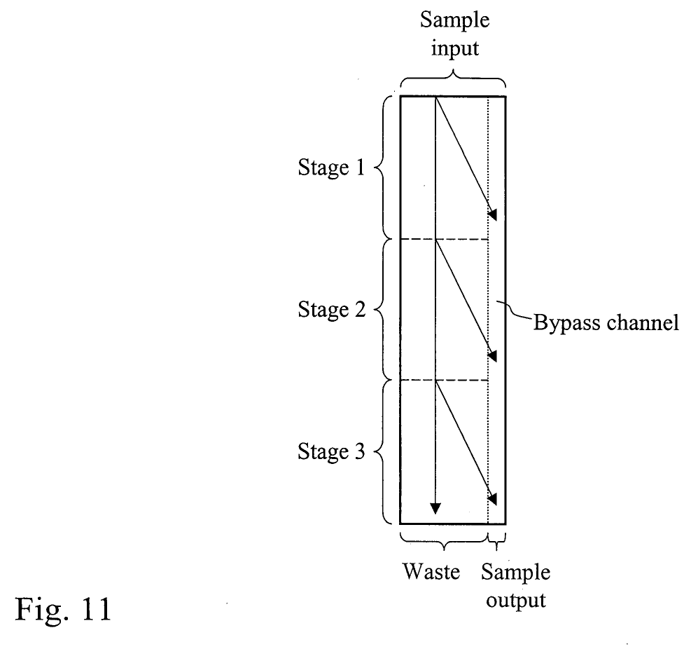

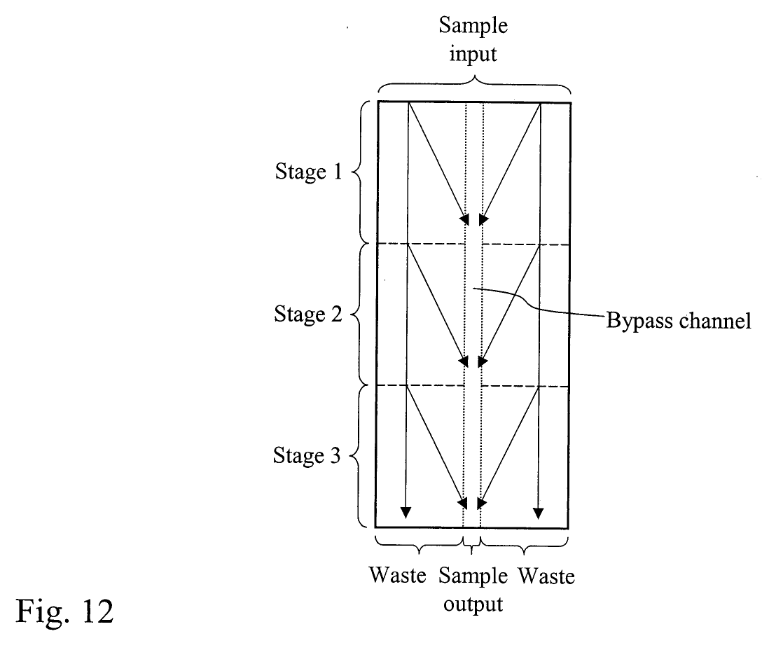

Single bypass channels. In this design, all stages share one bypass channel, or there is only one stage. The physical boundary of the bypass channel may be defined by the array boundary on one side and a sidewall on the other (FIGS. 9-11). Single bypass channels may also be employed with duplex arrays (FIG. 12).

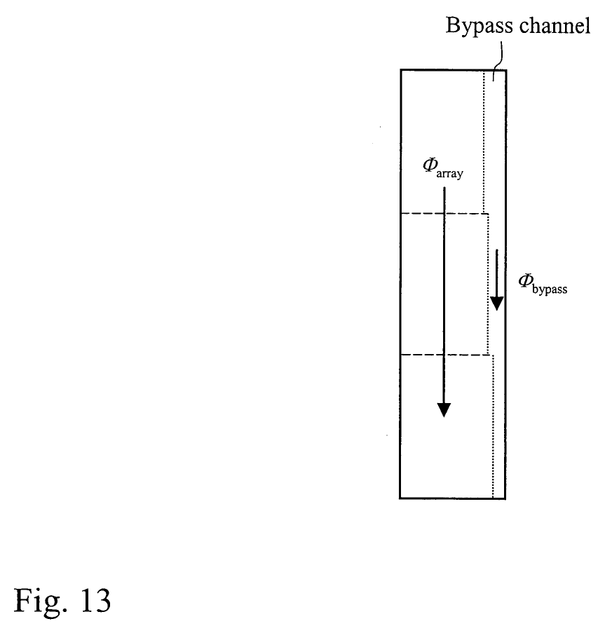

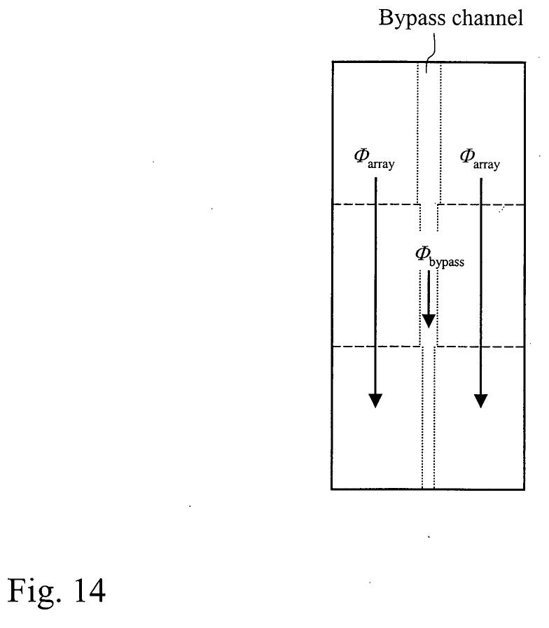

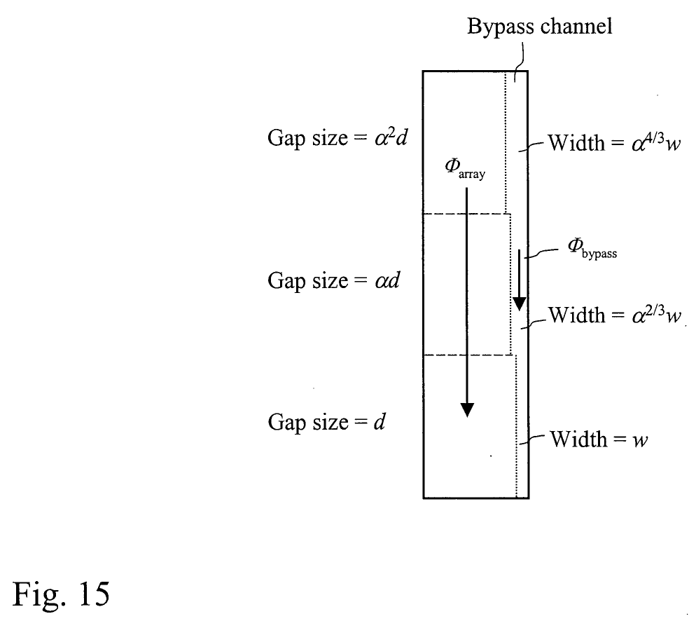

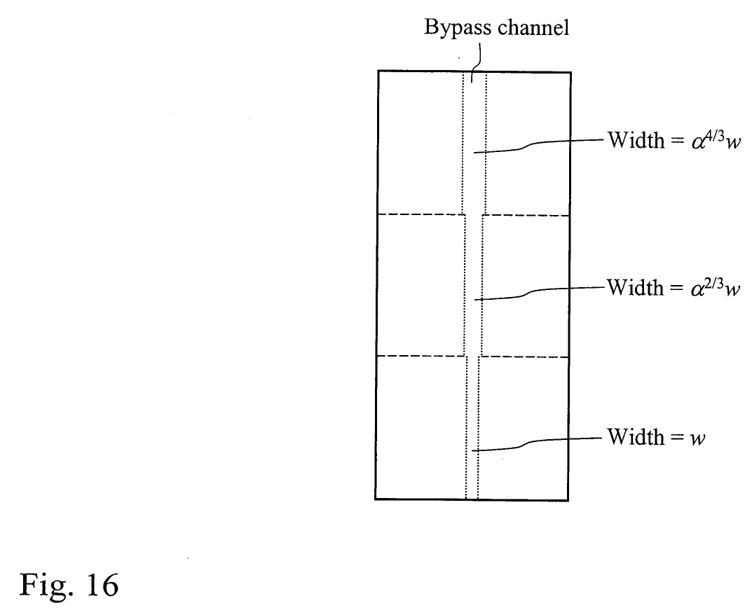

Single bypass channels may also be designed, in conjunction with an array, to maintain constant flux through a device (FIG. 13). As shown, the bypass channel has varying width designed maintain constant flux through all the stages, so that the flow in the channel does not interfere with the flow in the arrays. Such a design may also be employed with an array duplex (FIG. 14). Single bypass channels may also be designed in conjunction with the array in order to maintain substantially constant fluidic resistance through all stages (FIG. 15). Such a design may also be employed with an array duplex (FIG. 16.)

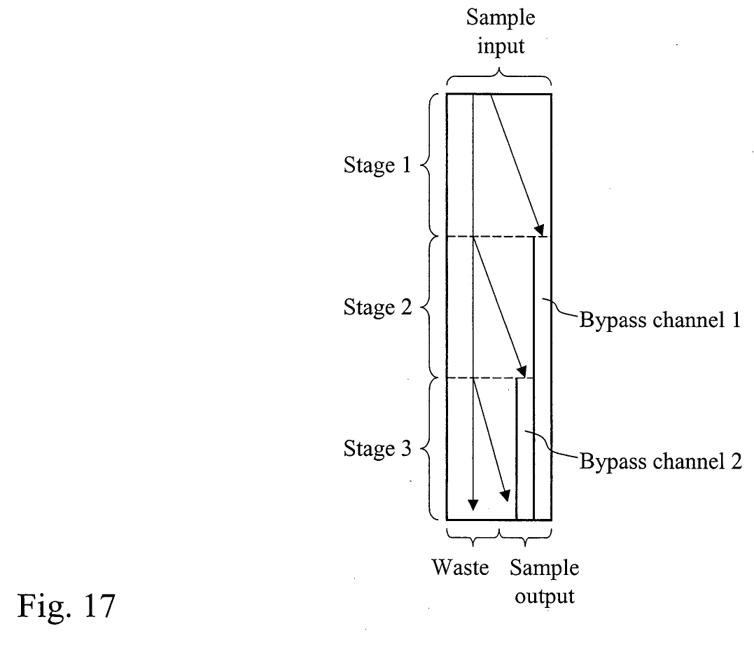

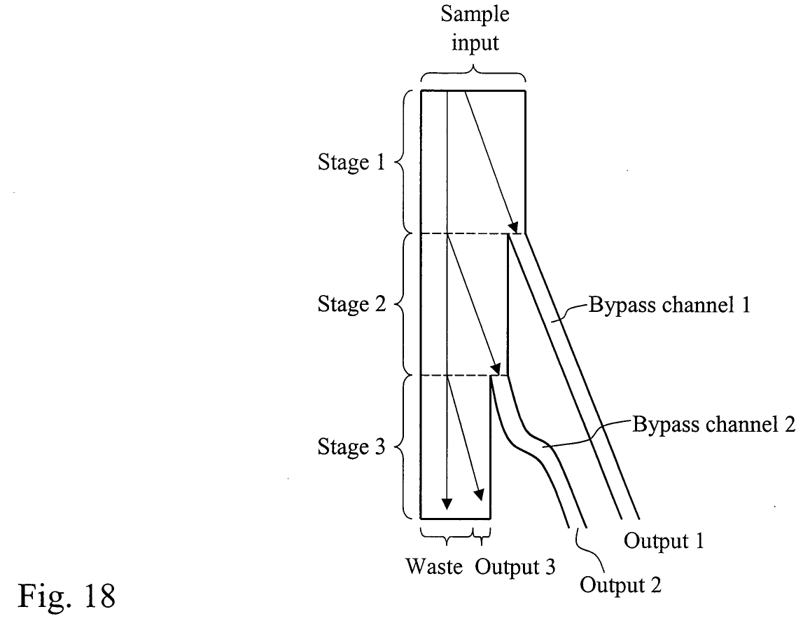

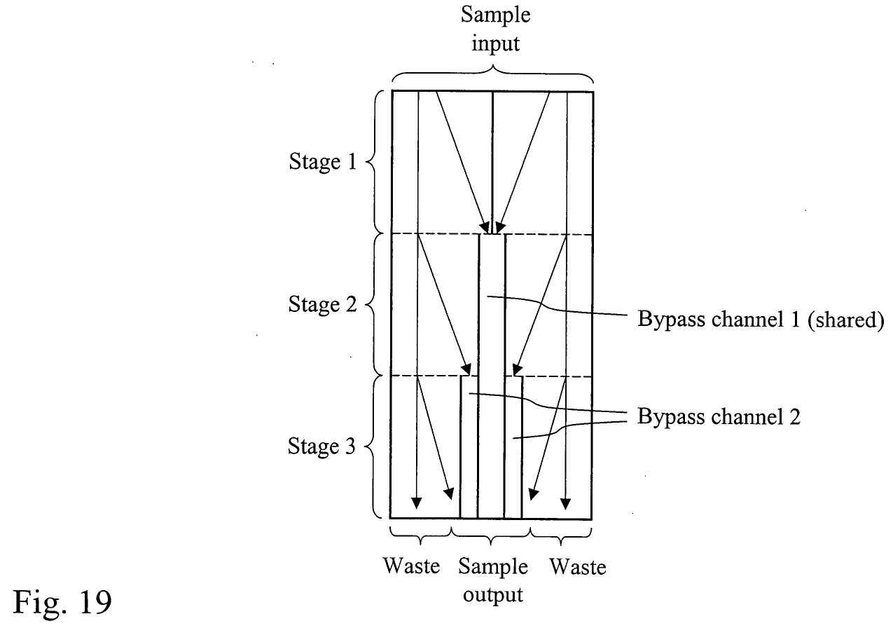

Multiple bypass channels. In this design (FIG. 17), each stage has its own bypass channel, and the channels are separated from each other by sidewalls. Large analytes, e.g., cells are deflected into the major flux to the lower right corner of the first stage and then into in the bypass channel (bypass channel 1 in FIG. 17). Smaller cells that would not cause clogging in the second stage proceed to the second stage, and cells above the critical size of the second stage are deflected to the lower right corner of the second stage and into in another bypass channel (bypass channel 2 in FIG. 17). This design may be repeated for as many stages as desired. In this embodiment, the bypass channels are not fluidically connected, allowing for collection or other manipulation of multiple fractions. The bypass channels do not need to be straight or be physically parallel to each other (FIG. 18). Multiple bypass channels may also be employed with duplex arrays (FIG. 19).

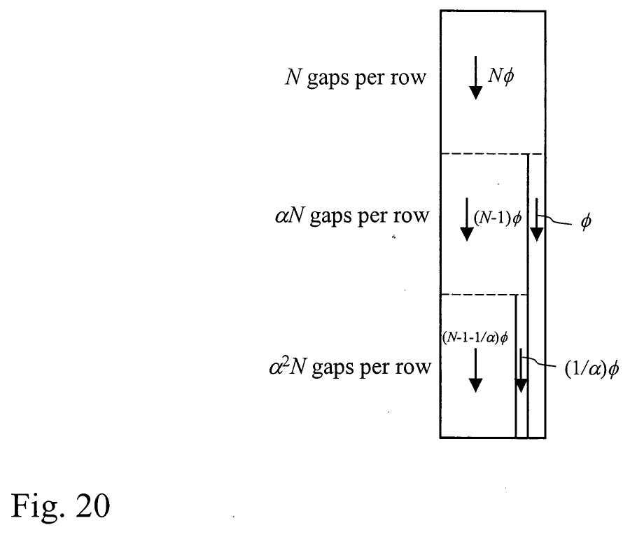

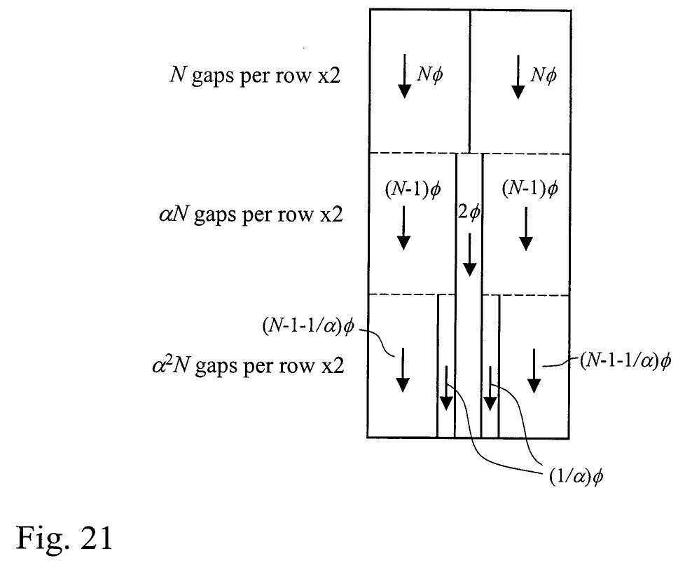

Multiple bypass channels may be designed, in conjunction with an array to maintain constant flux through a device (FIG. 20). In this example, bypass channels are designed to remove an amount of flow so the flow in the array is not perturbed, i.e., substantially constant. Such a design may also be employed with an array duplex (FIG. 21). In this design, the center bypass channel may be shared between the two arrays in the duplex.

Optimal boundary design. If the array were infinitely large, the flow distribution would be the same at every gap. The flux φ going through a gap would be the same, and the minor flux would be εφ for every gap. In practice, the boundaries of the array perturb this infinite flow pattern. Portions of the boundaries of arrays may be designed to generate the flow pattern of an infinite array. Boundaries may be flow-feeding, i.e., the boundary injects fluid into the array or flow-extracting, i.e., the boundary extracts fluid from the array.

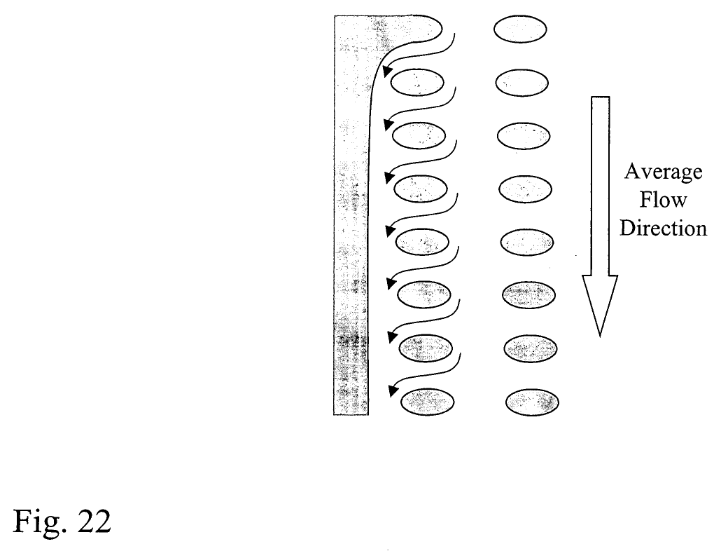

A preferred flow-extracting boundary widens gradually to extract εφ (represented by arrows in FIG. 22) from each gap at the boundary (d=24 μm, ε=1/60). For example, the distance between the array and the sidewall gradually increases to allow for the addition of εφ from each gap to the boundary. The flow pattern inside this array is not affected by the bypass channel because of the boundary design.

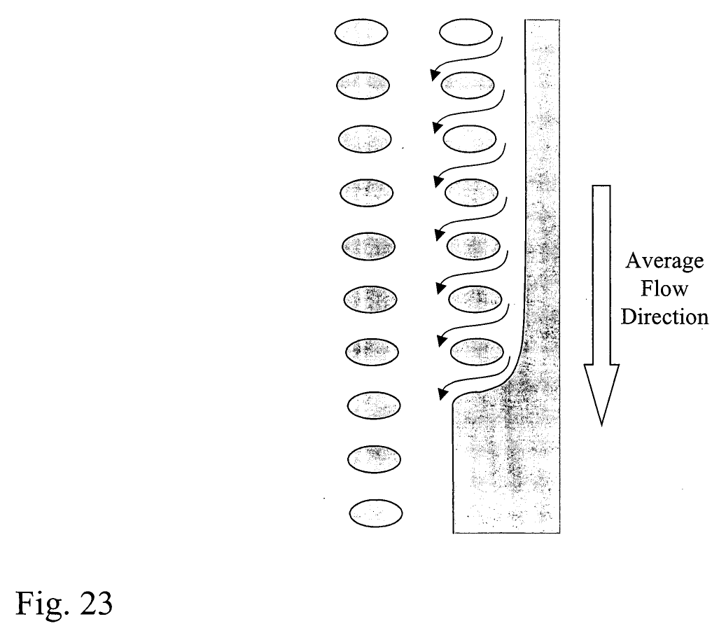

A preferred flow-feeding boundary narrows gradually to feed exactly εφ (represented by arrows in FIG. 23) into each gap at the boundary (d=24 μm, ε=1/60). For example, the distance between the array and the sidewall gradually decreases to allow for the removal of εφ to each gap from the boundary. Again, the flow pattern inside this array is not affected by the bypass channel because of the boundary design.

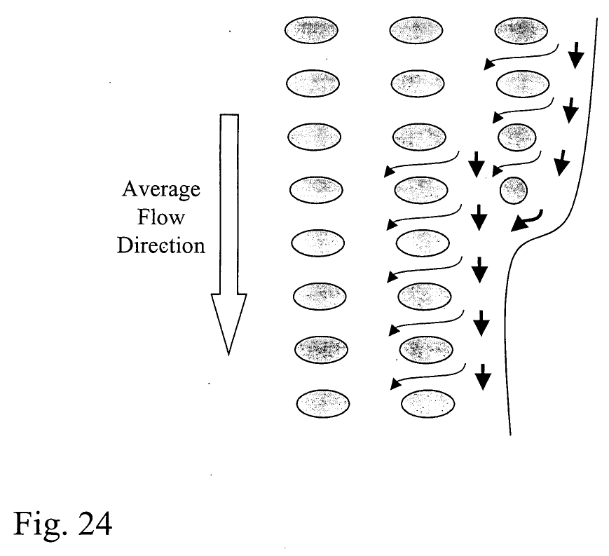

A flow-feeding boundary may also be as wide as or wider than the gaps of an array (FIG. 24) (d=24 μm, ε=1/60). A wide boundary may be desired if the boundary serves as a bypass channel, e.g., to allow for collection of analytes. A boundary may be employed that uses part of its entire flow to feed the array and feeds εφ into each gap at the boundary (represented by arrows in FIG. 24).

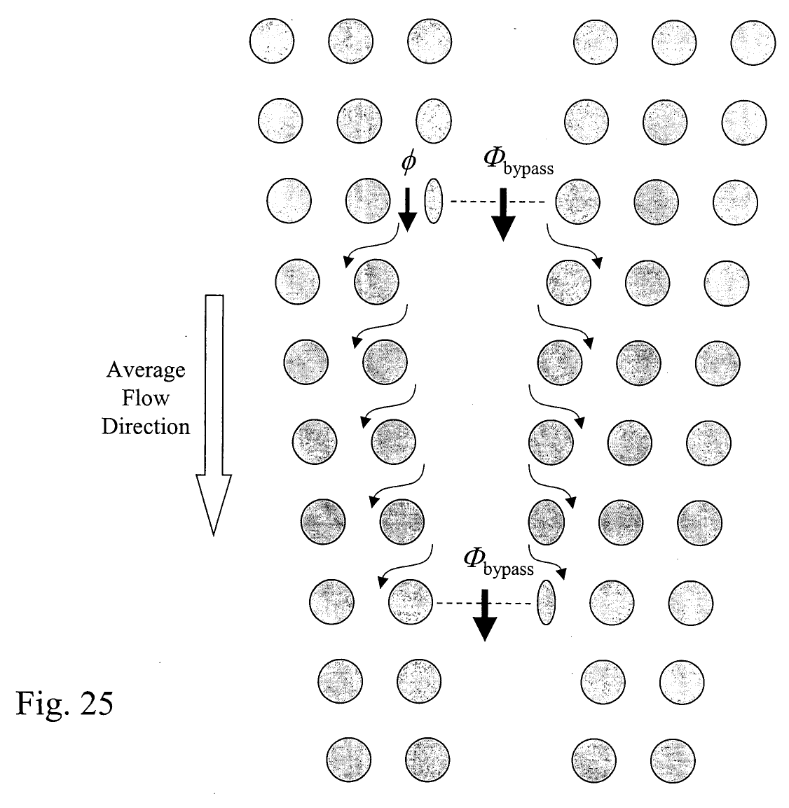

FIG. 25 shows a single bypass channel in a duplex array (ε=1/10, d=8 μm). The bypass channel includes two flow-feeding boundaries. The flux across the dashed line 1 in the bypass channel is Φ_{bypass}. A flow φ joins Φ_{bypass} from a gap to the left of the dashed line. The shapes of the obstacles at the boundaries are adjusted so that the flows going into the arrays are εφ at each gap at the boundaries. The flux at dashed line 2 is again Φ_{bypass}.

On-Chip Flow Resistor for Defining and Stabilizing Flow

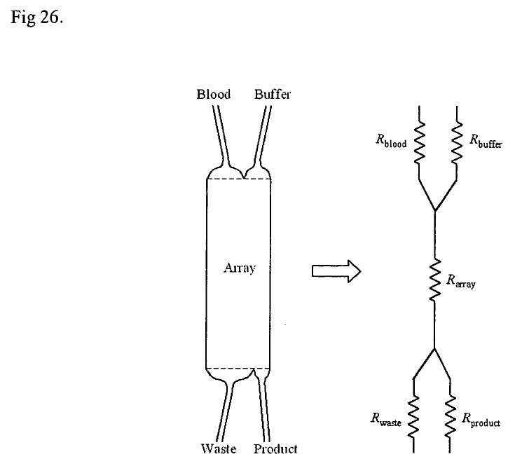

Deterministic separation may also employ fluidic resistors to define and stabilize flows within an array and to also define the flows collected from the array. FIG. 26 shows a schematic of planar device; a sample, e.g., blood, inlet channel, a buffer inlet channel, a waste outlet channel, and a product outlet channel are each connected to an array. The inlets and outlets act as flow resistors. FIG. 26 also shows the corresponding fluidic resistances of these different device components.

Flow Definition Within the Array

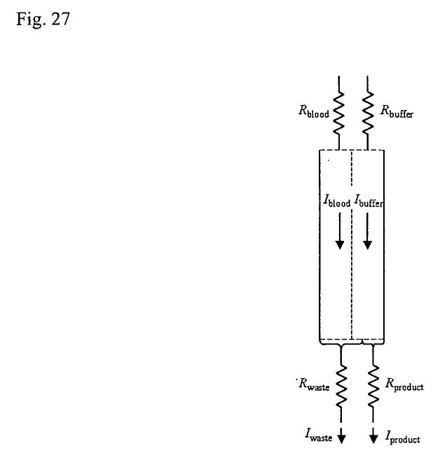

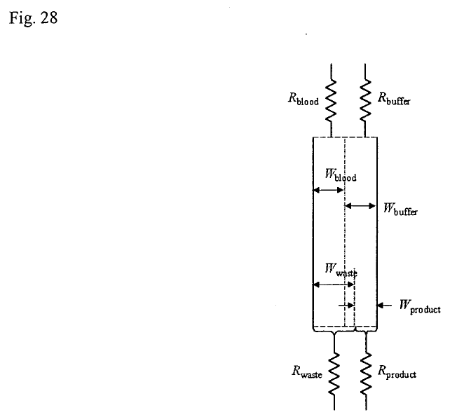

FIGS. 27 and 28 show the currents and corresponding widths of the sample and buffer flows within the array when the device has a constant depth and is operated with a given pressure drop. The flow is determined by the pressure drop divided by the resistance. In this particular device, I_{blood} and I_{buffer} are equivalent, and this determines equivalent widths of the blood and buffer streams in the array.

Definition of Collection Fraction

By controlling the relative resistance of the product and waste outlet channels, one can modulate the collection tolerance for each fraction. For example, in this particular set of schematics, when R_{product} is greater than R_{waste}, a more concentrated product fraction will result at the expense of a potentially increased loss to and dilution of waste fraction. Conversely, when R_{product} is less than R_{waste}, a more dilute and higher yield product fraction will be collected at the expense of potential contamination from the waste stream.

Flow Stabilization

Each of the inlet and outlet channels can be designed so that the pressure drops across the channels are appreciable to or greater than the fluctuations of the overall driving pressure. In typical cases, the inlet and outlet pressure drops are 0.001 to 0.99 times the driving pressure.

Multiplexed Deterministic Arrays

Deterministic separation may be achieved using multiplexed deterministic arrays. Putting multiple arrays on one device increases sample-processing throughput, and allows for parallel processing of multiple samples or portions of the sample for different fractions or manipulations. Multiplexing is further desirable for preparative applications. The simplest multiplex device includes two devices attached in series, i.e., a cascade. For example, the output from the major flux of one device may be coupled to the input of a second device. Alternatively, the output from the minor flux of one device may be coupled to the input of the second device.

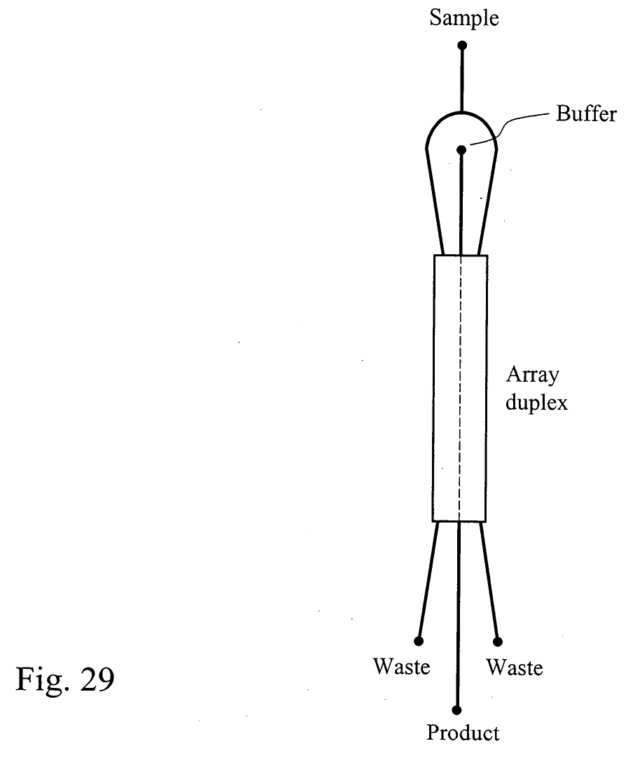

Duplexing. Two arrays can be disposed side-by-side, e.g., as mirror images (FIG. 29). In such an arrangement, the critical size of the two arrays may be the same or different. Moreover, the arrays may be arranged so that the major flux flows to the boundary of the two arrays, to the edge of each array, or a combination thereof. Such a multiplexed array may also contain a central region disposed between the arrays, e.g., to collect analytes above the critical size or to alter the sample, e.g., through buffer exchange, reaction, or labeling.

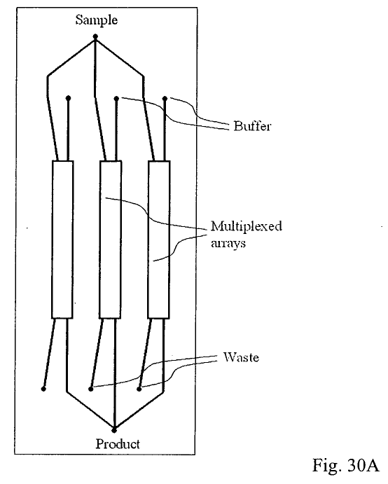

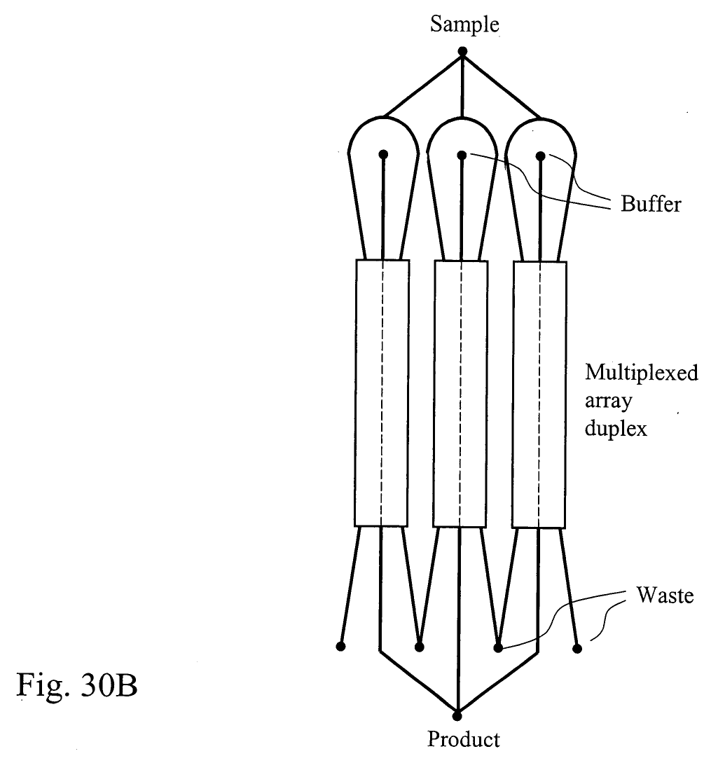

Multiplexing on a device. In addition to forming a duplex, two or more arrays that have separated inputs may be disposed on the same device (FIG. 30A). Such an arrangement could be employed for multiple samples, or the plurality of arrays may be connected to the same inlet for parallel processing of the same sample. In parallel processing of the same sample, the outlets may or may not be fluidically connected. For example, when the plurality of arrays has the same critical size, the outlets may be connected for high throughput samples processing. In another example, the arrays may not all have the same critical size or the analytes in the arrays may not all be treated in the same manner, and the outlets may not be fluidically connected.

Multiplexing may also be achieved by placing a plurality of duplex arrays on a single device (FIG. 30B). A plurality of arrays, duplex or single, may be placed in any possible three-dimensional relationship to one another.

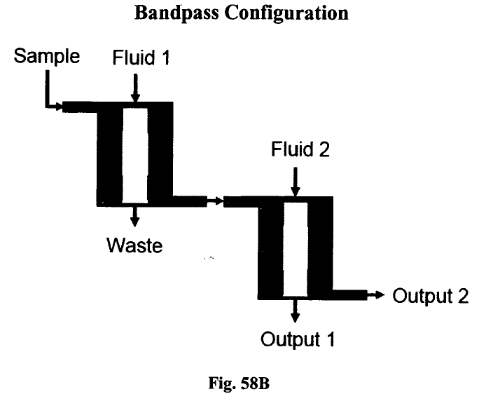

Exemplary multiple stage devices. In addition to those described above, the following exemplary multiple stage deterministic devices may also be included in devices of the invention. For example, FIG. 58A shows the “cascade” configuration, in which outlet 1 of one device is joined to a sample inlet of a second device. This allows for an initial separation step using the first device so that the sample introduced to the second device is already enriched for cells of interest. The two devices may have either identical or different critical sizes, depending on the intended application.

In FIG. 60, an unlabeled cellular sample is introduced to the first device in the cascade via a sample inlet, and a buffer containing labeling reagent is introduced to the first device via the fluid inlet. Epithelial cells are deflected and emerge from the center outlet in the buffer containing labeling reagent. This enriched labeled sample is then introduced to the second device in the cascade via a sample inlet, while buffer is added to the second device via the fluid inlet. Further enrichment of target cells and separation of free labeling reagent is achieved, and the enriched sample may be further analyzed. Alternatively, labeling reagent may be added directly to the sample emerging from the center outlet of the first device before introduction to the second device. The use of a cascade configuration may allow for the use of a smaller quantity or a higher concentration of labeling reagent at less expense than the single-device configuration of FIG. 60; in addition, any nonspecific binding that may occur is significantly reduced by the presence of an initial separation step using the first device.

An alternative configuration of two or more device stages is the “bandpass” configuration. FIG. 58B shows this configuration, in which outlet 2 of one device is joined to a sample inlet of a second device. This allows for an initial separation step using the first device so that the sample introduced to the second device contains cells that remained undeflected within the first device. This method may be useful when the cells of interest are not the largest cells in the sample; in this instance, the first stage may be used to reduce the number of large non-target cells by deflecting them to the center outlet. As in the cascade configuration, the two devices may have either identical or different critical sizes, depending on the intended application. For example, different critical sizes are appropriate for an application requiring the separation of epithelial cells, in comparison with an application requiring the separation of smaller endothelial cells.

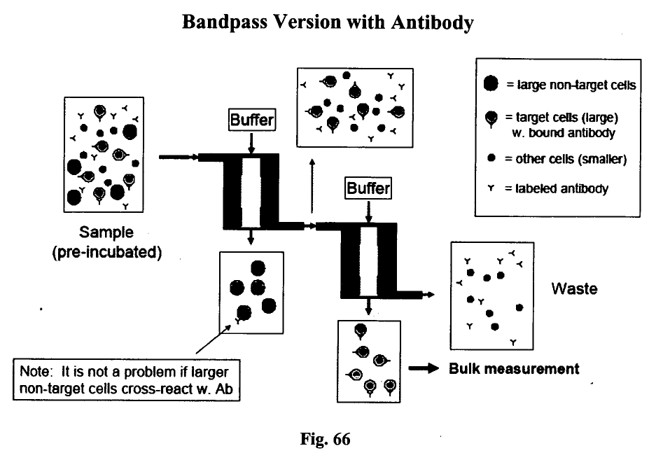

In FIG. 66, a cellular sample pre-incubated with labeling reagent is introduced to a sample inlet of the first device of the bandpass configuration, and a buffer is introduced to the first device via the fluid inlet. The first device is disposed in such a manner that large, non-target cells are deflected and emerge from the center outlet, while a mixture of target cells, small non-target cells, and labeling reagent emerge from outlet 2 of the first device. This mixture is then introduced to the second device via a sample inlet, while buffer is added to the second device via the fluid inlet. Enrichment of target cells and separation of free labeling reagent is achieved, and the enriched sample may be further analyzed. Non-specific binding of labeling reagent to the deflected cells in the first stage is acceptable in this method, as the deflected cells and any bound labeling reagent are removed from the system.

In any of the multiple deterministic device configurations described above, the devices and the connections joining them may be integrated into a single device. For example, a single cascade device including two or more stages is possible, as is a single bandpass device including two or more stages. The output of the multiple stages is then coupled to the input of the reservoir.

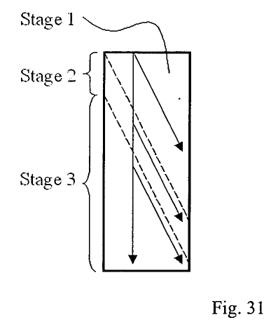

Small-footprint arrays. Deterministic devices may also feature a small footprint. Reducing the footprint of an array can lower cost, and reduce the number of collisions with obstacles to eliminate any potential mechanical damage or other effects to analytes. The length of a multiple stage array can be reduced if the boundaries between stages are not perpendicular to the direction of flow. The length reduction becomes significant as the number of stages increases. FIG. 31 shows a small-footprint three-stage array.

Uses of Devices of the Invention

As described, the invention features devices and methods for the enrichment of analytes such as particles, including bacteria, viruses, fungi, cells, cellular components, viruses, nucleic acids, proteins, and protein complexes. Examples of fluid samples that are contemplated by the present invention include biological fluid samples, such as, whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal and genitourinary tracts, and amniotic fluid. Moreover, any other biological sample (e.g., a biopsy sample) which may be solubilized or suspended is also contemplated by the systems and methods herein. In addition to enrichment, a device may also be used to effect various manipulations on analytes in a sample. Such manipulations include alteration of the analyte itself, e.g., a magnetic property, or the fluid carrying the analyte. Preferably, a device is employed to enrich rare analytes from a heterogeneous mixture or to alter a rare analyte, e.g., by exchanging the liquid in the sample or by contacting an analyte with a reagent. Such devices allow for a high degree of enrichment with limited stress on a potentially fragile analyte such a cell, where devices of the invention provide reduced mechanical lysis or intracellular activation of cells.

Although primarily described in terms of cells, the devices of the invention may be employed with any analyte capable of being isolated, enriched, or depleted based on a magnetic property (or lack thereof) and/or other methods of enrichment described herein, e.g., enrichment based on hydrodynamic size.

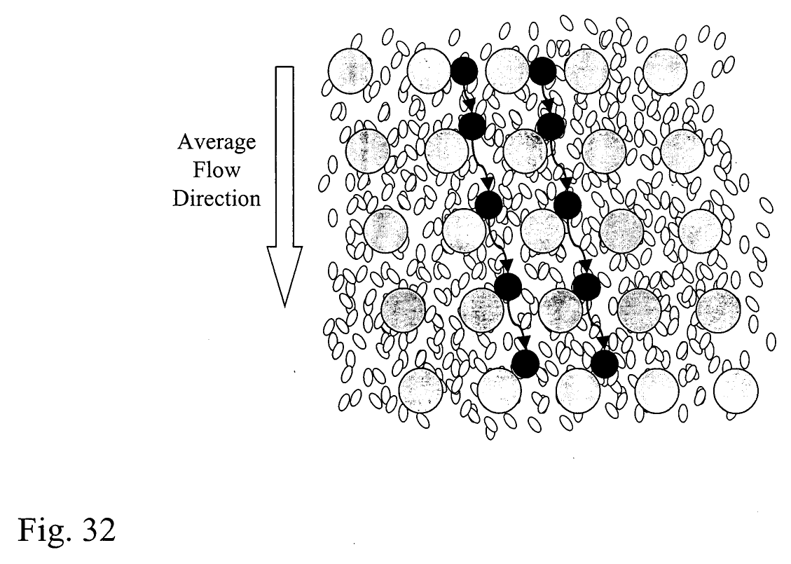

Deterministic devices, and other analytical devices, may be employed in concentrated samples, e.g., where analytes are touching, hydrodynamically interacting with each other, or exerting an effect on the flow distribution around another analyte. For example, a deterministic device can separate white blood cells from red blood cells in whole blood from a human donor. Human blood typically contains ˜45% of cells by volume. Cells are in physical contact and/or coupled to each other hydrodynamically when they flow through the array. FIG. 32 shows schematically that cells densely packed inside an array can physically interact with each other.

As described, the devices and methods of the invention may involve separating from a sample one or more analytes based on an intrinsic or extrinsic magnetic property of the one or more analytes. In one embodiment, the sample is treated with a reagent that alters a magnetic property of the analyte. The alteration may be mediated by a magnetic particle or may be mediated by a reagent that alters an intrinsic magnetic property of the analyte. A magnetically responsive analyte may then be attracted to a surface of the device, and desired analytes (e.g., rare cells such as fetal cells, pathogenic cells, cancer cells, or bacterial cells) in a sample may be retained in the device. In another embodiment, desired analytes are retained in the device through size-, shape- or deformability-based mechanisms. In another embodiment, negative selection is employed, where an undesired, magnetically susceptible analyte is bound in the device while the desired analyte is not. In addition to binding, the path of a magnetically susceptible analyte may be altered by a magnetic field, e.g., to direct desired, or undesired, analytes into a specified direction, e.g., towards an outlet. Any of the embodiments may use a MACS column for retention of an analyte (e.g., an analyte bound to a magnetic particle).

In embodiments of the invention using positive selection, it is desirable that at least 20%, 30%, 40%, 50%, 60%, 70%, 80%, 90%, 95%, 98%, or 99% of the target analytes are retained in the enriched sample, e.g., magnetically bound to the device. The surfaces of the device are desirably designed to minimize nonspecific binding of non-target analytes. Furthermore, at least 99%, 98%, 95%, 90%, 80%, or 70% of non-target analytes are preferably not retained in the enriched sample, e.g., not magnetically bound to the device. The selective retention in the device can result in the separation of a specific analyte population from a mixture, e.g., blood, sputum, urine, and soil, air, or water samples.

The methods and devices of the invention allow for the production of an enriched sample of high purity, e.g., where at least 0.01%, 0.1%, 1%, 10%, 20%, 50%, 60%, 70%, 80%, 90%, or even 95% of the enriched sample is the desired analyte. High purity is particularly desirable when the analyte is a cell, e.g., a fetal red blood cell or an epithelial cell, as it allows for use of quantitative PCR methods. The devices of the invention also allow for high purity with high yield (i.e., retention of the desired analyte). For example, at least 90% of desired analytes, e.g., fetal red blood cells, present in a sample are retained in a sample enriched by the devices of the invention, and at least 90%, e.g., at least 95% or even 99.9%, of undesired analytes, e.g., white bloods cells, are not retained in the enriched sample.

In additional embodiments, devices of the invention can be used for isolation and detection of blood borne pathogens, bacterial and viral loads, airborne pathogens solubilized or suspended in aqueous medium, pathogen detection in food industry, and environmental sampling for chemical and biological hazards. Additionally, the magnetic particles can be co-localized with a capture moiety and a candidate drug compound. Capture of a cell of interest can further be analyzed for the interaction of the captured cell with the immobilized drug compound. A device can thus be used to both isolate sub-populations of cells from a complex mixture and assay their reactivity with candidate drug compounds for use in the pharmaceutical drug discovery process for high throughput and secondary cell-based screening of candidate compounds. In other embodiments, receptor-ligand interaction studies for drug discovery can be accomplished in the device by localizing the capture moiety, i.e., the receptor, on a magnetic particle, and flowing in a complex mixture of candidate ligands (or agonists or antagonists). The ligand of interest is captured, and the binding event can be detected, e.g., by secondary staining with a fluorescent probe. This embodiment enables rapid identification of the absence or presence of known ligands from complex mixtures extracted from tissues or cell digests or identification of candidate drug compounds.

Magnetic particles. The selective retention of analytes may be obtained by introduction of magnetic particles (e.g., attached to obstacles present in the device or manipulated to create obstacles to increase surface area for an analyte to interact with to increase the likelihood of binding) into a device of the invention. Capture moieties may be bound to the magnetic particles to effect specific binding of a target analyte. In another embodiment, the magnetic particles may be disposed such as to only allow analytes of a selected size, shape, or deformability to pass through the device. Combinations of these embodiments are also envisioned. For example, a device may be configured to retain certain analytes based on size and others based on binding. In addition, a device may be designed to bind more than one analyte of interest, e.g., in a serial, parallel, or interspersed arrangement of regions within a device or where two or more capture moieties are disposed on the same magnetic particle or on adjacent particles, e.g., bound to the same obstacle or region. Further, multiple capture moieties that are specific for the same analyte (e.g., anti-CD71 and anti-CD36) may be employed in the device, either on the same or different magnetic particles, e.g., disposed on the same or different obstacle or region.

The flow conditions in the device are typically such that the analytes are very gently handled in the device to prevent damage. Positive pressure or negative pressure pumping or flow from a column of fluid may be employed to transport analytes into and out of the microfluidic devices of the invention. The device enables gentle processing, while maximizing the collision frequency of each analyte with one or more of the magnetic particles. The target analytes interact with any capture moieties on collision with the magnetic particles. The capture moieties can be co-localized with obstacles as a designed consequence of the magnetic field attraction in the device. This interaction leads to capture and retention of the target analytes in defined locations. Captured analyte can be released by demagnetizing the magnetic regions retaining the magnetic particles. For selective release of analytes from regions, the demagnetization can be limited to selected obstacles or regions. For example, the magnetic field can be designed to be electromagnetic, enabling turn-on and turn-off off the magnetic fields for each individual region or obstacle at will. In other embodiments, the particles can be released by disrupting the bond between the analyte and the capture moiety, e.g., through chemical cleavage or interruption of a noncovalent interaction, or by decreasing the magnetic responsiveness of the bound analyte. For example, some ferrous particles are linked to monoclonal antibody via a DNA linker; the use of DNAse can cleave and release the analytes from the ferrous particle. Alternatively, an antibody fragmenting protease (e.g., papain) can be used to engineer selective release. Increasing the sheer forces on the magnetic particles can also be used to release magnetic particles from magnetic regions, especially hard magnetic regions. In other embodiments, the captured analytes are not released and can be analyzed or further manipulated while retained.

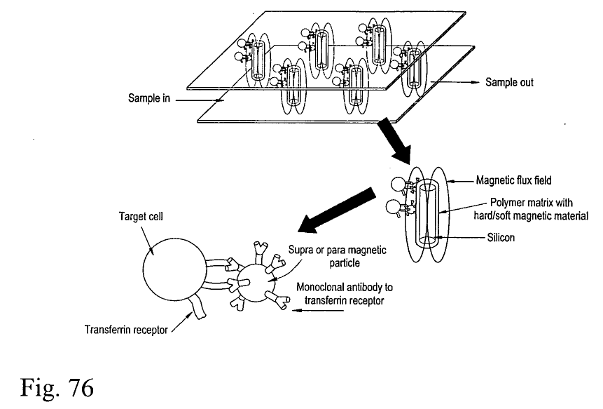

FIG. 76 illustrates an example of a reservoir designed to capture and isolate cells expressing the transferrin receptor from a complex mixture. Monoclonal antibodies to CD71 receptor are readily available off-the-shelf covalently coupled to magnetic materials, such as, but not limited to, ferrous doped polystyrene and ferroparticles or ferro-colloids (e.g., from Miltenyi and Dynal). The mAB to CD71 bound to magnetic particles is flowed into the reservoir. The antibody-coated particles are attracted to the obstacles (e.g., posts), floor, and walls and are retained by the strength of the magnetic field interaction between the particles and the magnetic field. The particles between the obstacles and those loosely retained with the sphere of influence of the local magnetic fields away from the obstacles, are removed by a rinse (the flow rate can be adjusted such that the hydrodynamic shear stress on the particles away from the obstacles is larger than the magnetic field strength).

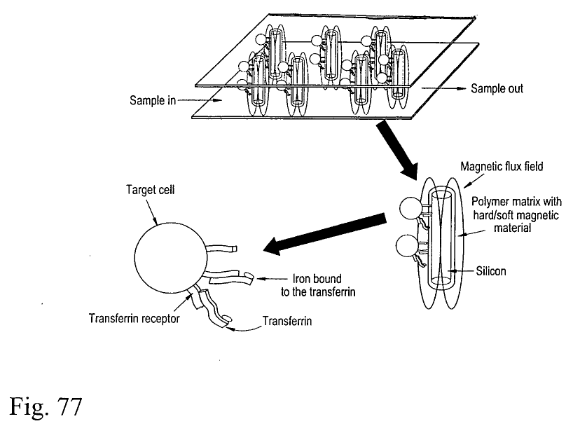

FIG. 77 is a preferred embodiment for application of the reservoir to capture and release CD71+ cells from a complex mixture, e.g., blood, using holo-transferrin. Holo-transferrin is rich in iron content, commercially available, and has higher affinity constants and specificity of interaction with the CD71 receptor than its counterpart monoclonal antibody. The iron coupled to the transferrin ligand serves the dual purpose of retaining the conformation of the ligand for binding with the cell receptor, and as a molecular paramagnetic element for retaining the ligand on the obstacles.

Enrichment

In one embodiment, devices of the invention are employed to produce a sample enriched in a desired analyte, e.g., based at least in part on a magnetic property, and optionally hydrodynamic size. Applications of such enrichment include concentrating of an analyte such as particle including rare cells. Devices may also be used to enrich components of cells such as organelles (e.g., nuclei). Desirably, the devices and methods of the invention retain at least 1%, 10%, 30%, 50%, 75%, 80%, 90%, 95%, 98%, or 99% of the desired analyte compared to the initial mixture, while potentially enriching the desired analytes by a factor of at least 1, 10, 100, 1,000, 10,000, 100,000, or even 1,000,000 relative to one or more non-desired analytes. The enrichment may also result in a dilution of the desired analytes compared to the original sample, although the concentration of the desired analytes relative to other analytes in the sample has increased. Preferably, the dilution is at most 90%, e.g., at most 75%, 50%, 33%, 25%, 10%, or 1%.

In another embodiment, a device of the invention is used to produce a sample enriched in a rare analyte. In general, a rare analyte is an analyte that is present as less than 10%, 9%, 8%, 7%, 6%, 5%, 4%, 3%, 2%, 1%, 0.1%, 0.01%, 0.001%, 0.0001%, 0.00001%, or 0.000001% of all analytes in a sample or whose mass is less than 10%, 9%, 8%, 7%, 6%, 5%, 4%, 3%, 2%, 1%, 0.1%, 0.01%, 0.001%, 0.0001%, 0.00001%, or 0.000001% of total mass of a sample. Exemplary rare analytes include, depending on the sample, fetal cells, bone marrow cells, progenitor cells, stem cells (e.g., undifferentiated), foam cells, cancer cells, immune system cells (host or graft), epithelial cells, endothelial cells, endometrial cells, trophoblasts, connective tissue cells, bacteria, fungi, viruses, and pathogens (e.g., bacterial or protozoa). Such rare analytes may be isolated from samples including bodily fluids, e.g., blood, or environmental sources, e.g., pathogens in water samples. Fetal red blood cells may be enriched from maternal peripheral blood, e.g., for the purpose of determining sex and identifying aneuploidies or genetic characteristics, e.g., mutations, in the developing fetus. Circulating tumor cells, which are typically of epithelial cell type and origin, may also be enriched from peripheral blood for the purpose of diagnosis and monitoring therapeutic progress. Circulating endothelial cells may also be similarly enriched from peripheral blood.

Bodily fluids or environmental samples may also be screened for pathogens, e.g., for coliform bacteria, blood borne illnesses such as sepsis, or bacterial or viral meningitis. Rare cells also include cells from one organism present in another organism, e.g., in cells from a transplanted organ.

The amount of blood, or other bodily fluid, drawn can vary depending on the mammal and its condition, e.g., stage of pregnancy or disease, e.g., cancer. In some embodiments, less than 50 mL, 40 mL, 30 mL, 20 μL, 10 mL, 9 mL, 8 mL, 7 mL, 6 mL, 5 mL, 4 mL, 3 mL, 2 mL, 1 mL, 0.5 mL, 0.1 mL, 0.05 mL, or even 0.01 mL of fluid are obtained from an individual. In some embodiments, 1-50 mL, 2-40 mL, 3-30 mL, or 4-20 mL of fluid are obtained from an individual. In other embodiments, more than 5, 10, 15, 20, 15, 30, 35, 40, 45, 50, 55, 60, 65, 70, 75, 80, 85, 90, 95 or 100 mL of fluid are obtained from an individual. For example, in some embodiments the systems and methods herein allow for the detection and isolation of a rare cell (e.g., fetal cell) from a maternal blood sample of less than 5 mL or 3 mL. In other examples, the systems and methods herein can be used to analyze or enrich rare cells from larger volumes of blood such as those greater than 20 mL or more than 50 mL. Any one of the above functions can occur within, for example, less than 1 day, or 12, 10, 11, 9, 8, 7, 6, 5, 4, 3, 2, or 1 hours. An entire sample collected can be applied to the apparatus herein for enrichment and/or detection of rare cells. Alternatively, the sample may be processed such that only certain component are introduced into a device.

In addition to enrichment of a rare analyte, a device may be employed for preparative applications. An exemplary preparative application includes generation of cell packs from blood. In one example, a device may be configured to produce fractions enriched in platelets, red blood cells, and white cells by magnetic separation, alone or in conjunction with deterministic enrichment. By using multiplexed or multistage devices, all three cellular fractions may be produced in parallel or in series from the same sample. In other embodiments, the device may be employed to separate nucleated from non-nucleated cells, e.g., from cord blood sources.

Devices of the invention are advantageous in situations where the analytes being enriched are subject to damage or other degradation. As described herein, devices may be designed to enrich analytes (e.g., a cell) with a minimum number of collisions between the analyte and obstacles or other surfaces. This minimization reduces mechanical damage to the analytes (e.g., a cell) and, in the case of cells, also prevents or reduces intracellular activation caused by the collisions. Gentle handling preserves the limited number of rare analytes in a sample, in the case of cells, prevents or reduces rupture leading to contamination or degradation by intracellular components, and prevents or reduces maturation or activation of cells, e.g., stem cells or platelets. In preferred embodiments, the analyte is enriched such that fewer than 30%, 10%, 5%, 1%, 0.1%, or even 0.01% are damaged (e.g., activated or mechanically lysed).

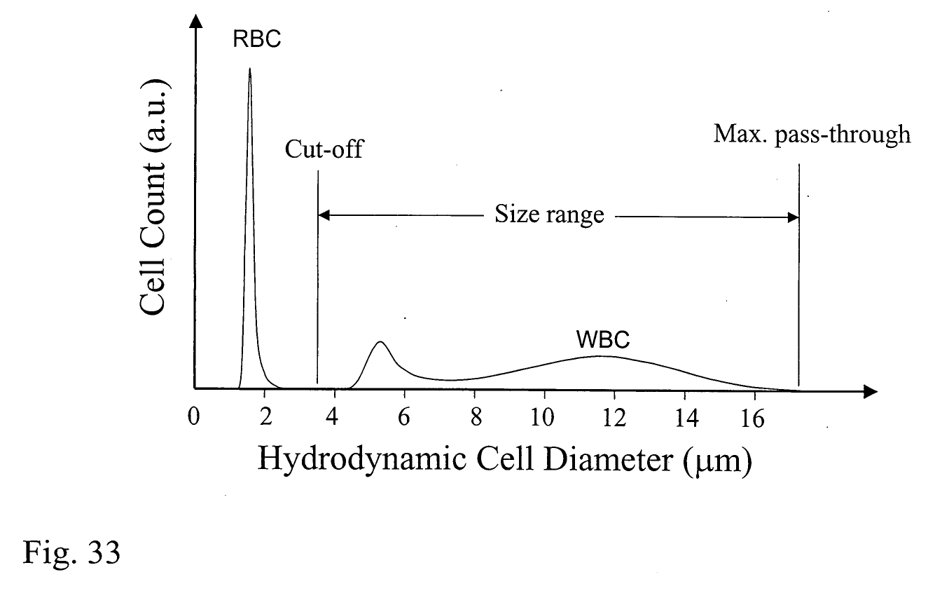

FIG. 33 shows a typical size distribution of cells in human peripheral blood. The white blood cells range from ˜4 μm to ˜18 μm, whereas the red blood cells are ˜1.5 μm (short axis). A deterministic device designed to separate white blood cells from red blood cells typically has a cut-off size of 2 to 4 μm and a maximum pass-through size of greater than 18 μm. Such a device may be used in conjunction with magnetic separation as described herein.

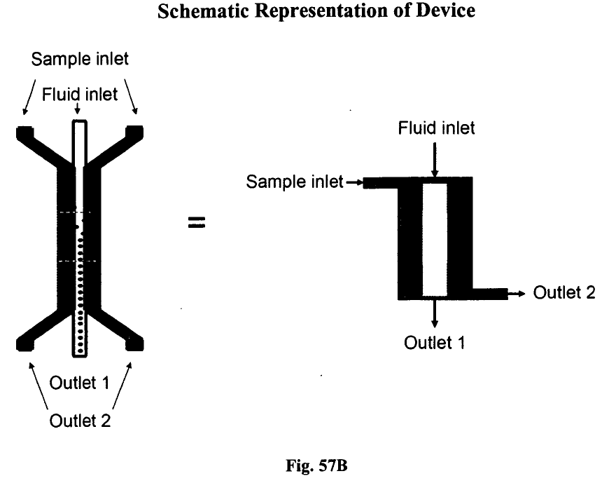

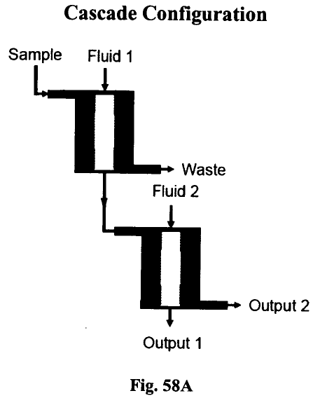

FIG. 57A shows the operation of a deterministic device for purposes of enrichment. A cellular sample is added through a sample inlet of the device, and buffer medium is added through the fluid inlet. Cells below the critical size move through the device undeflected, emerging from the edge outlets in their original sample medium. Cells above the critical size, e.g., epithelial cells, are deflected and emerge from the center outlet contained in the buffer medium added through the fluid inlet. Operation of the device thus produces samples enriched in cells above and below the critical size. Because epithelial cells are among the largest cells in the bloodstream, the size and geometry of the gaps of the device may be chosen so as to direct virtually all other cell types to the edge outlets, while producing a sample from the center outlet that is substantially enriched in epithelial cells after a single pass through the device.

A deterministic device included in the invention need not be duplexed as shown in FIG. 57A in order to operate as described herein. The schematized representation shown in FIG. 57B may represent either a duplexed device or a single array.

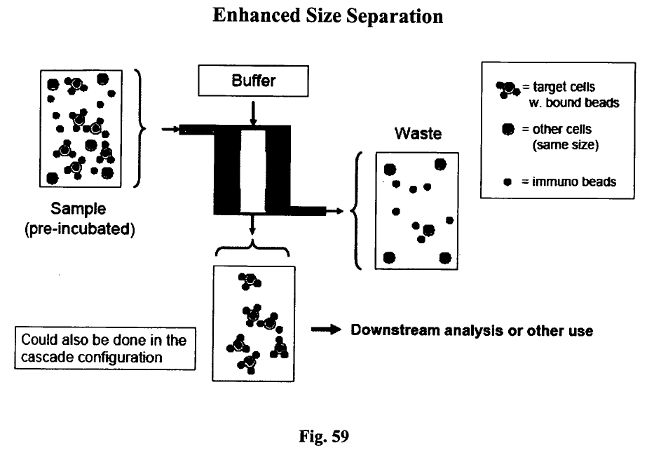

Enrichment may be enhanced in numerous ways by coupling magnetic separation with deterministic separation. For example, target analytes (e.g., cells) may be labeled with beads (e.g., immunoaffinity beads), thereby increasing their size (as depicted in FIG. 59) and potentially also altering the magnetic properties of the analytes. In the case of epithelial cells, this may further increase their size, resulting in an even more efficient separation. Alternatively, the size of smaller analytes (e.g., cells) may be increased to the extent that they become the largest objects in the sample or occupy a unique size range in comparison to the other components of the sample, or so that they copurify with other analytes. Beads may be made of polystyrene, magnetic material, or any other material that can be adhered to an analyte (e.g., cells). Desirably, such beads are neutrally buoyant so as not to disrupt the flow of labeled cells through a device. Modification of the size of analytes may be used in enrichment based magnetic and deterministic properties employed in parallel or in series.

Alteration

In other embodiments, in addition to or in the absence of enrichment, an analyte of interest may be contacted with an altering reagent that may chemically or physically alter the analyte or the fluid in the sample. Applications include purification, buffer exchange, labeling (e.g., immunohistochemical, magnetic, and histochemical labeling, cell staining, and flow in-situ fluorescence hybridization (FISH)), magnetic alteration, cell fixation, cell stabilization, cell lysis, and cell activation.

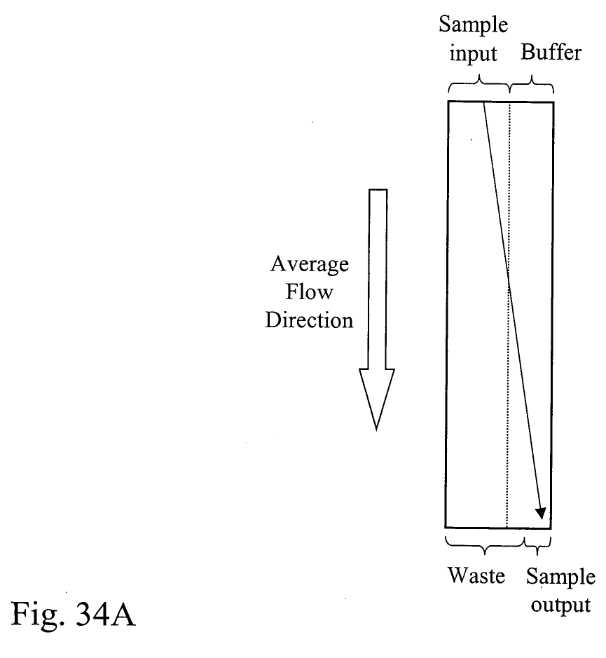

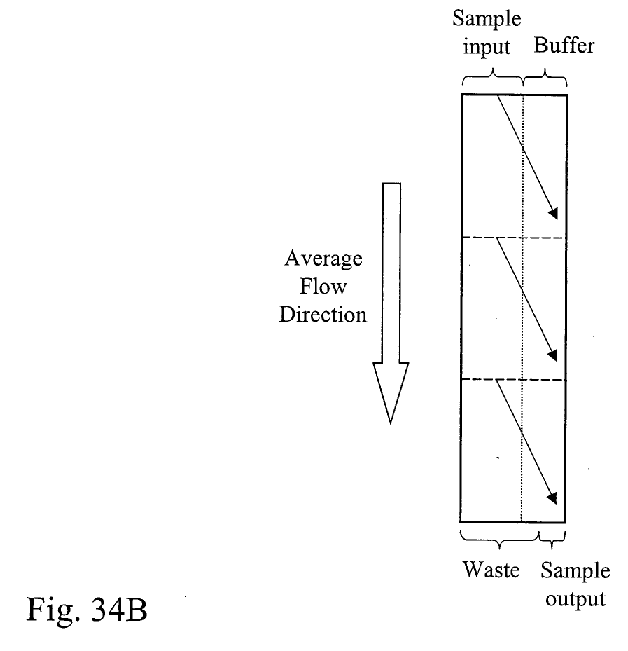

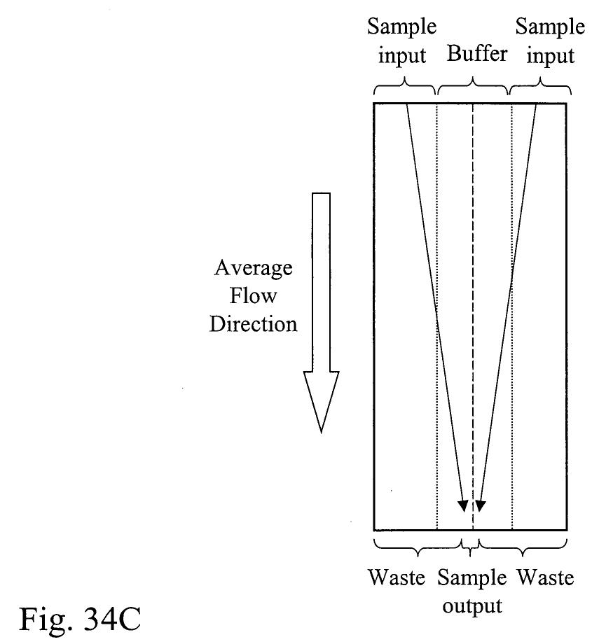

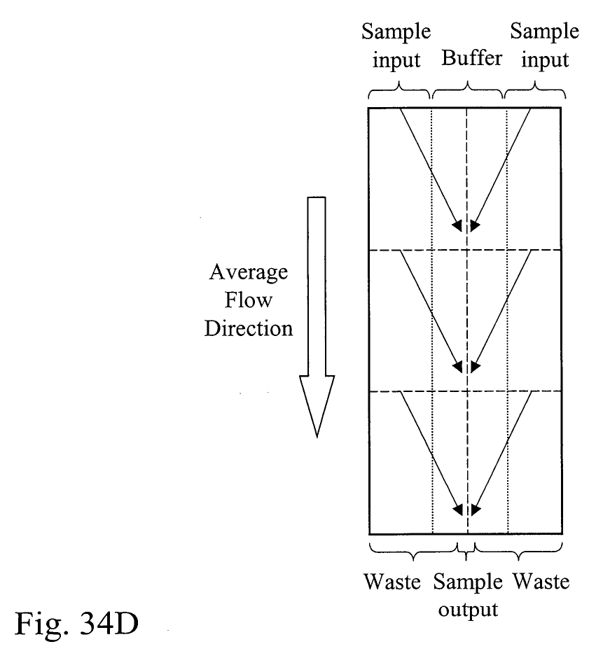

Such methods may allow for the transfer of analytes from a sample into a different liquid (e.g., buffer exchange). FIG. 34A shows this effect schematically for a single stage deterministic device, FIG. 34B shows this effect for a multistage deterministic device, FIG. 34C shows this effect for a duplex array of deterministic devices, and FIG. 34D shows this effect for a multistage duplex array of deterministic devices. Similarly, magnetic separation may be employed to retain an analyte in a device, or deflect it in a desired direction, to effect buffer exchange. By using such methods, analytes (e.g., blood cells) may be enriched in the sample. Such transfers of an analyte from one liquid to another may be also employed to effect a series of alterations, e.g., Wright staining blood on-chip. Such a series may include reacting an analyte with a first reagent and then transferring the analyte to a wash buffer, and then another reagent.

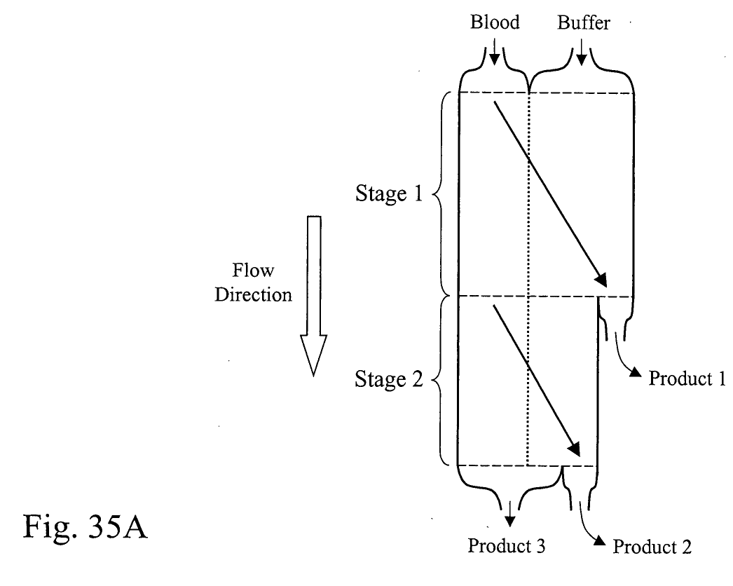

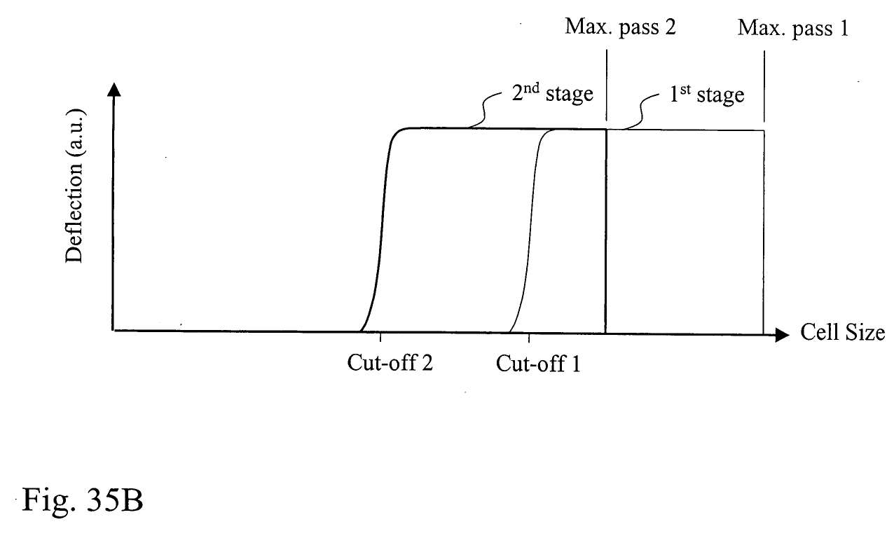

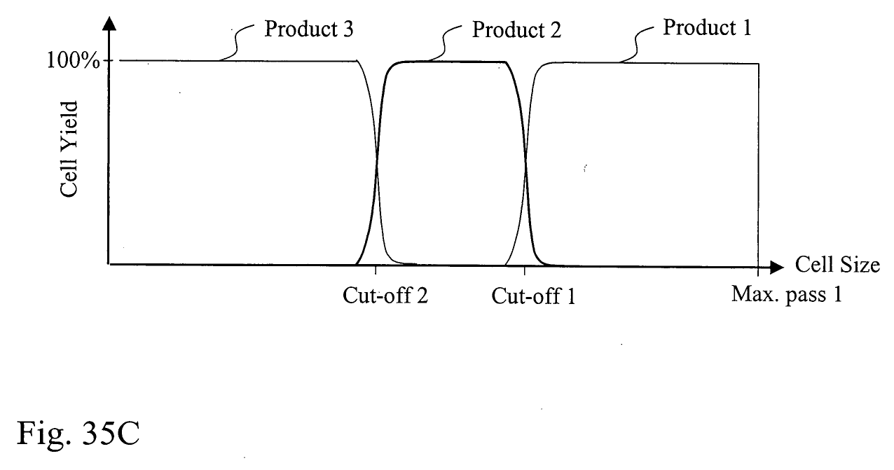

FIGS. 35A-35C illustrates a further example of alteration in a two stage deterministic device having two bypass channels. In this example, the larger analytes are moved from blood to buffer (e.g., containing a reagent that alters a magnetic property of the analyte) and collected in stage 1, intermediate sized analytes are moved from blood to buffer (e.g., containing a reagent that alters a magnetic property of the analyte) in stage 2, and smaller analytes that are not moved from the blood in stage are collected also collected. FIG. 35B illustrates the size cut-off of the two stages, and FIG. 35C illustrates the size distribution of the three fractions collected. The collected fractions may then be subjected to magnetic based enrichment.

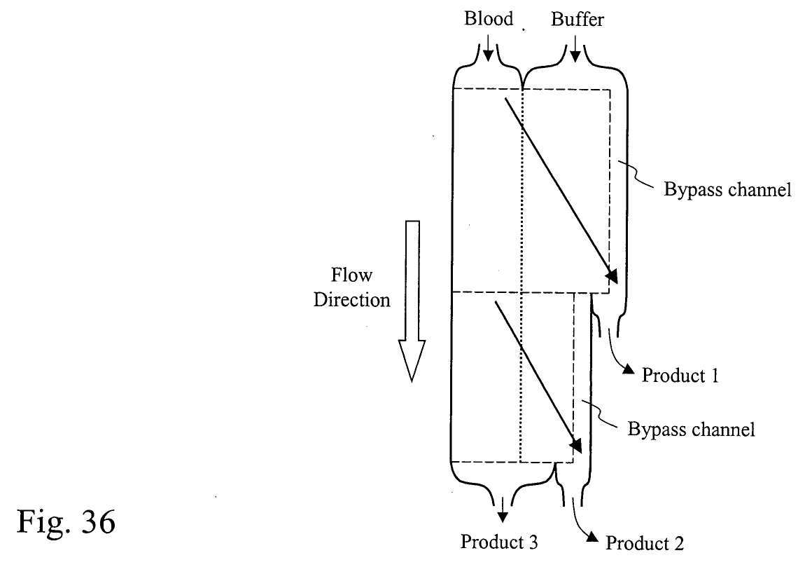

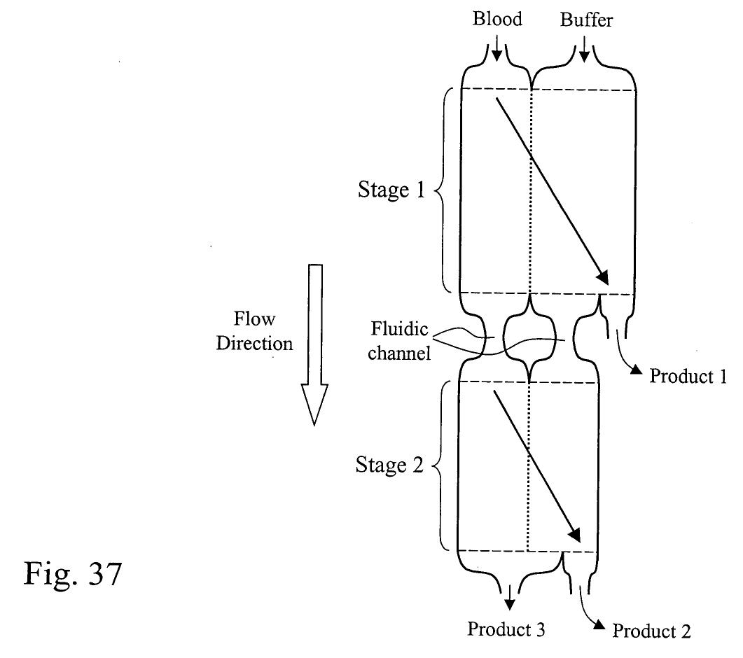

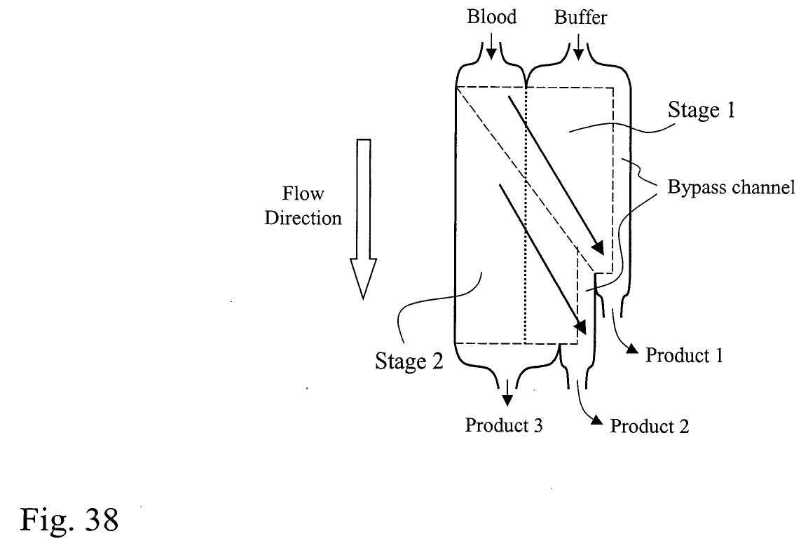

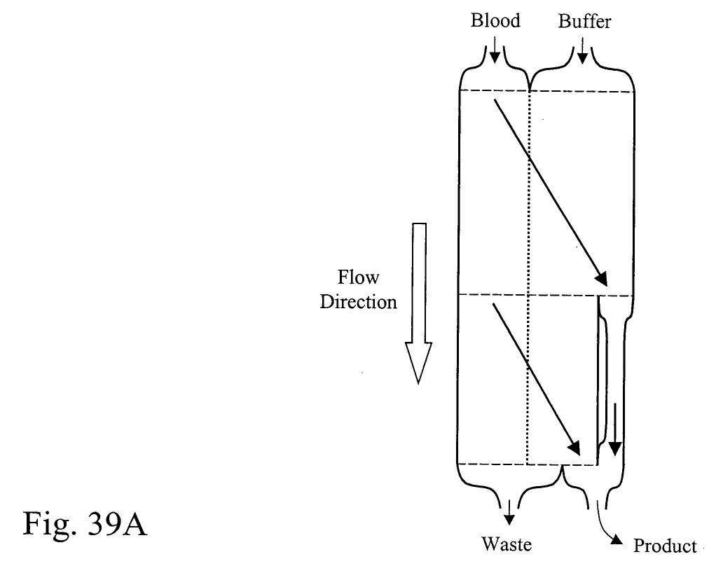

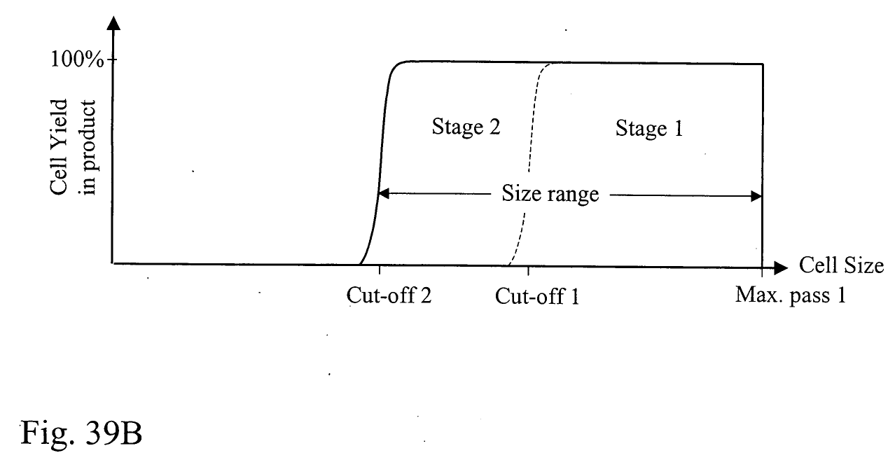

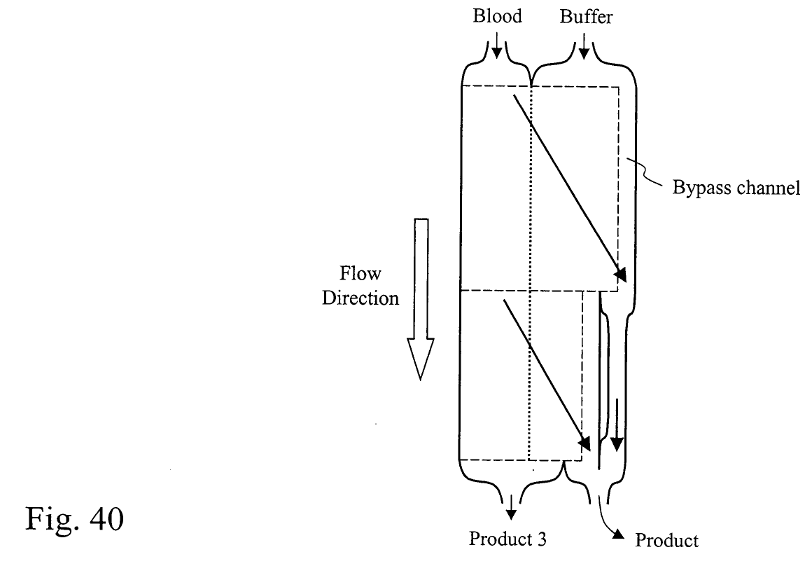

FIG. 36 illustrates an example of alteration, e.g., of a magnetic property, in a two stage deterministic device having bypass channels that are disposed between the lateral edge of the array and the channel wall. FIG. 37 illustrates a deterministic device similar to that in FIG. 36, except that the two stages are connected by fluidic channels. FIG. 38 illustrates alteration in a deterministic device having two stages with a small footprint. FIGS. 39A-39B illustrates alteration in a device in which the output from the first and second stages is captured in a single channel. FIG. 40 illustrates another device for use in the methods of the invention.

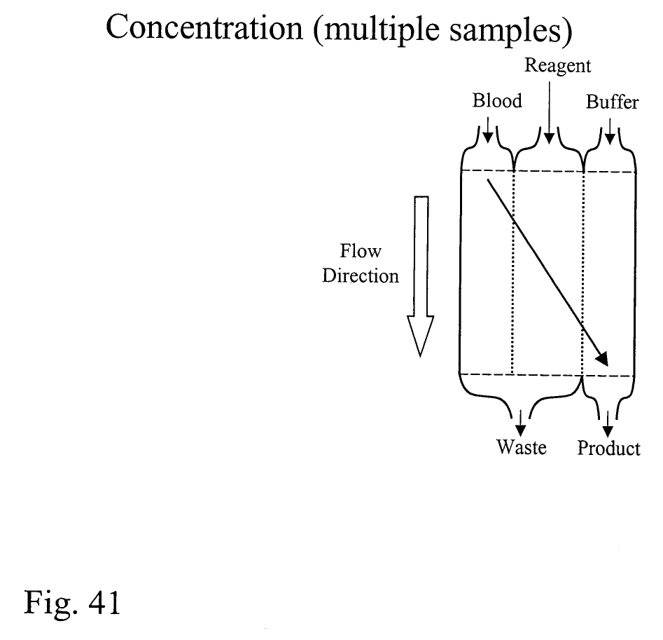

FIG. 41 illustrates the use of a deterministic device to perform multiple, sequential alterations on an analyte. In this device an analyte is moved from the sample into a regent that reacts with the analyte, and the altered analyte is then moved into a buffer, thereby removing the unreacted reagent or reaction byproducts. Additional steps may be added (e.g., steps described herein).

Enrichment and alteration may also be combined. For example, desired cells may be contacted with a lysing reagent and cellular components, e.g., nuclei, are enriched based on size, magnetic properties, or both. In another example, analytes may be contacted with particulate labels, e.g., magnetic beads, which bind to the analytes. Unbound particulate labels may be removed based on size, magnetic properties, or both.

Concentration

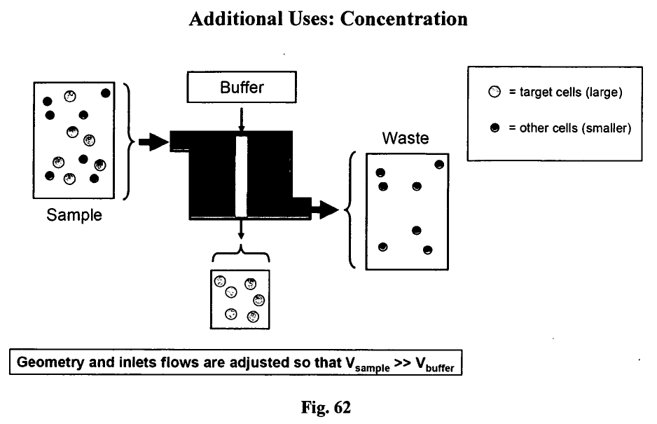

Devices of the invention may also be employed in order to concentrate a sample, e.g., of cells, of interest. In one example shown in FIG. 62, a cellular sample is introduced to the sample inlet of a deterministic device. By reducing the volume of buffer introduced into the fluid inlet so that this volume is significantly smaller than the volume of the cellular sample, concentration of target cells in a smaller volume results. Similarly, retaining a magnetically responsive analyte in a channel may be employed to concentrate the analyte, e.g., by releasing the retained analyte in a smaller volume. This concentration step may improve the results of any downstream analysis performed.

Cell Lysis

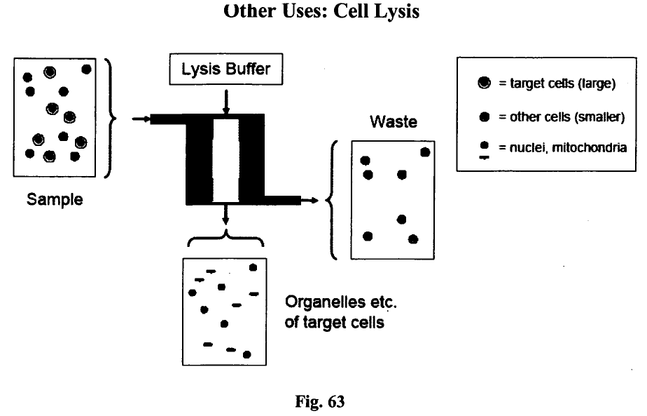

Devices of the invention may also be employed for purposes of cell lysis. To achieve this in a deterministic device, a protocol similar to that used for enrichment is followed: a cellular sample is added through a sample inlet of the device (FIG. 63), and lysis buffer is added through the fluid inlet. As described above, cells above the critical size are deflected and enter the lysis buffer, leading to lysis of these cells. As a result, the sample emerging from the center outlet includes lysed cell components including organelles, while undeflected whole cells emerge from the other outlet. Similarly, cells that are retained (or not retained) in a device based on a magnetic property may be contacted with a lysing reagent, e.g., to release intracellular components of analytes magnetically bound. Thus, the device provides a method for selectively lysing target cells.

Downstream Analysis

The enriched analytes, e.g., rare cell and/or components, can be detected using any means known in the art. For example, in some embodiments a detection module herein includes an imager, e.g., a microscope, camera, spectrometer, or hyperspectral imager (see, e.g., Vo-Dinh et al., IEEE Eng. Med. Biol. Mag. 23:40-49 (2004)). Detection may involve the use of preferential staining and detection of color changes, which indicate the presence or absence of an analyte of interest. In some embodiments, the staining strategy for cell identification will employ an indirect immunostaining approach using an unlabeled primary antibody followed by a secondary enzyme conjugated antibody. Exemplary enzymes include horseradish peroxidase and alkaline phosphatase. Chromogenic stains are generated from well known colorless substrates for the conjugated enzymes. In some embodiments identified rare cells which have been stained are further analyzed for chromosomal abnormalities by nucleic acid hybridization using specific probes. This triage strategy preferably utilizes one stain for the rare cells and a different marker on a nucleic acid probe.

A key prerequisite for many diagnostic assays is the removal or reduction below a threshold level of a free or unreacted altering reagent from the sample to be analyzed. In one embodiment, the reagent is a labeling reagent. As described above, devices of the invention are able to separate free labeling reagent from labeling reagent bound to an analyte (e.g., a cell). It is then possible to perform a bulk measurement of the reacted sample without significant levels of background interference from free labeling reagent. In one example, fluorescent antibodies selective for a particular epithelial cell marker such as EpCAM are used. The fluorescent moiety may include Cy dyes, Alexa dyes, or other fluorophore-containing molecules. The resulting labeled sample is then analyzed by measuring the fluorescence of the resulting sample of labeled enriched analytes such as cells using a fluorimeter. Alternatively, a chromophore-containing label may be used in conjunction with a spectrometer. The measurements obtained may be used to quantify the number of target analytes such as cells in a sample.

Many other methods of measurement and labeling reagents are useful in the methods and devices of the invention. Labeling antibodies may possess covalently bound enzymes that cleave a substrate, altering its absorbance at a given wavelength; the extent of cleavage is then quantified with a spectrometer. Colorimetric or luminescent readouts are possible, depending on the substrate used. Advantageously, the use of an enzyme label allows for significant amplification of the measured signal, lowering the threshold of detectability.

Quantum dots, e.g., Qdots® from QuantumDot Corp., may also be utilized as a labeling reagent that is covalently bound to a capture moiety such as an antibody. Qdots are resistant to photobleaching and may be used in conjunction with two-photon excitation measurements.

Another possible labeling reagent useful in the methods of the invention is phage. Phage display is a technology in which binding peptides are displayed by engineered phage strains having strong binding affinities for a target, e.g., a protein found on the surface of cells of interest. The peptide sequence corresponding to a given phage is encoded in that phage's nucleic acid. Thus, phage are useful labeling reagents in that they are potentially small relative to an analyte such as a cell and thus may be easily separated, and they additionally carry nucleic acid that may be analyzed and quantified using PCR or similar techniques, enabling a quantitative determination of the number of cells present in an enriched sample.

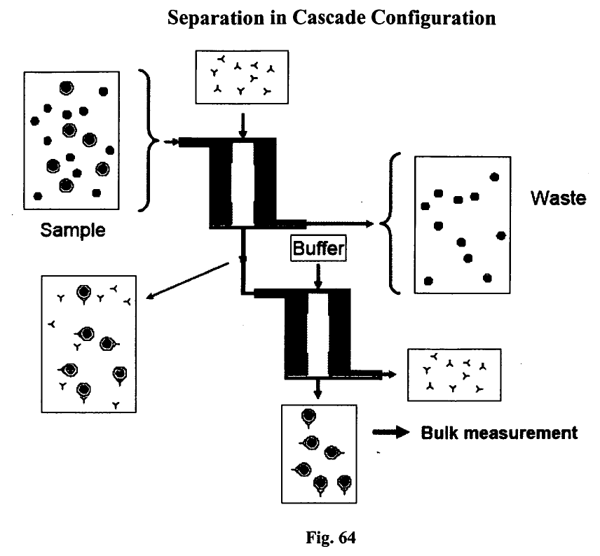

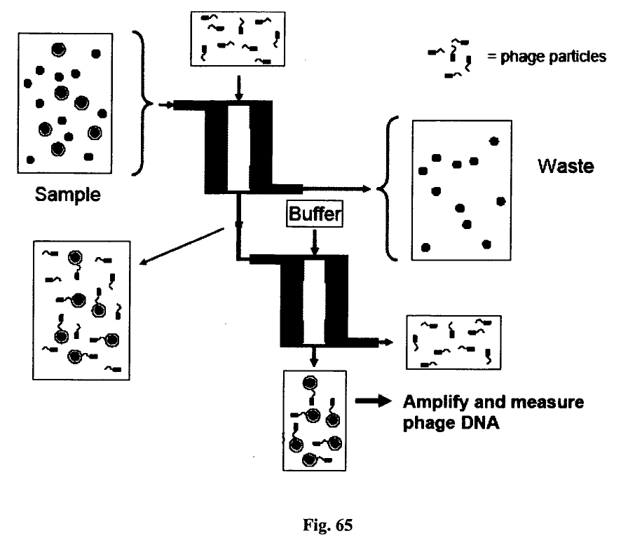

FIG. 65 depicts the use of phage as a labeling reagent in which two deterministic device stages are arrayed in a cascade configuration. The method depicted in FIG. 65 fits the general description of FIG. 64, with the exception of the labeling reagent employed. Magnetic enrichment may be similarly employed.

Downstream analysis may include an accurate determination of the number of desired analytes (e.g., cells) in the sample being analyzed. In order to produce accurate quantitative results, the amount of the target of a labeling reagent (e.g., a surface antigen on a cell of interest) typically has to be known or predictable (e.g., based on expression levels in a cell), and the binding of the labeling reagent should also proceed in a predictable manner, e.g., free from interfering substances. Thus, a device or method that produces a highly enriched cellular samples prior to introduction of a labeling reagent is particularly useful. In addition, labeling reagents that allow for amplification of the signal produced are preferred in the case of a rare desired analyte (e.g., epithelial cells in a blood sample). Reagents that allow for signal amplification include enzymes and phage. Other labeling reagents that do not allow for convenient amplification but nevertheless produce a strong signal, such as quantum dots, are also desirable. Quantification may also occur with an unaltered or unlabeled analyte.

When the devices and methods of the invention are used to enrich cells contained in a sample, further quantification and molecular biology analysis may be performed on the same set of cells. The gentle treatment of the cells in the devices of the invention, coupled with the described methods of bulk measurement, maintain the integrity of the cells so that further analysis may be performed if desired. For example, techniques that destroy the integrity of the cells may be performed subsequent to bulk measurement; such techniques include DNA or RNA analysis, proteome analysis, or metabolome analysis. An example of such analysis is PCR, in which the cells are lysed and levels of particular DNA sequences are amplified. Such techniques are particularly useful when the number of target cells isolated is very low.

Cancer Diagnosis

Epithelial cells exfoliated from solid tumors have been found in the circulation of patients with cancers of the breast, colon, liver, ovary, prostate, and lung. In general, the presence of circulating tumor cells (CTCs) after therapy has been associated with tumor progression and spread, poor response to therapy, relapse of disease, and/or decreased survival. Therefore, enumeration of CTCs offers a means to stratify patients for baseline characteristics that predict initial risk and subsequent risk based upon response to therapy.

Unlike tumor-derived cells in bone marrow, which can be dormant and long-lived, CTCs, which are of epithelial cell type and origin, have a short half-life of approximately one day, and their presence indicates a recent influx from a proliferating tumor (Patel et al., Ann Surg, 235:226-231, 2002). Therefore, CTCs can reflect the current clinical status of patient disease and therapeutic response. The enumeration and characterization of CTCs has potential value in assessing cancer prognosis and in monitoring therapeutic efficacy for early detection of treatment failure that can lead to disease relapse. In addition, CTC analysis may detect early relapse in presymptomatic patients who have completed a course of therapy; at present, individuals without measurable disease are not eligible to participate in clinical trials of promising new treatments (Braun et al., N Engl J Med, 351:824-826, 2004).

The devices and methods of the invention may be used to evaluate cancer patients and those at risk for cancer. For example, a blood sample is drawn from the patient and introduced to a device of the invention to separate epithelial cells from other blood cells. The number of epithelial cells in the blood sample is determined, e.g., using a method described herein. For example, the cells may be labeled with an antibody that binds to EpCAM, and the antibody may have a covalently bound fluorescent label, or be bound to a magnetic particle. A bulk measurement may then be made of the enriched sample produced by the device, and from this measurement, the number of epithelial cells present in the initial blood sample may be determined. Microscopic techniques may be used to visually quantify the cells in order to correlate the bulk measurement with the corresponding number of labeled cells in the blood sample.

By making a series of measurements over days, weeks, months, or years, one may track the level of epithelial cells present in a patient's bloodstream as a function of time. In the case of existing cancer patients, this provides a useful indication of the progression of the disease and assists medical practitioners in making appropriate therapeutic choices based on the increase, decrease, or lack of change in circulating epithelial cells in the patient's bloodstream. For those at risk of cancer, a sudden increase in the number of cells detected may provide an early warning that the patient has developed a tumor. This early diagnosis, coupled with subsequent therapeutic intervention, is likely to result in an improved patient outcome in comparison to an absence of diagnostic information.

Diagnostic methods include making bulk measurements of labeled epithelial cells isolated from blood, as well as techniques that destroy the integrity of the cells. For example, PCR may be performed on a sample in which the number of target cells isolated is very low; by using primers specific for particular cancer markers, information may be gained about the type of tumor from which the analyzed cells originated. Additionally, RNA analysis, proteome analysis, or metabolome analysis may be performed as a means of diagnosing the type or types of cancer present in the patient.

One important diagnostic indicator for lung cancer and other cancers is the presence or absence of certain mutations in epidermal growth factor receptor (EGFR). EGFR consists of an extracellular ligand-binding domain, a transmembrane portion, and an intracellular tyrosine kinase (TK) domain. The normal physiologic role of EGFR is to bind ErbB ligands, including epidermal growth factor (EGF), at the extracellular binding site to trigger a cascade of downstream intracellular signals leading to cell proliferation, survival, motility and other related activities. Many non-small cell lung tumors with EGFR mutations respond to small molecule EGFR inhibitors, such as gefitinib (Iressa; AstraZeneca), but often eventually acquire secondary mutations that make them drug resistant. Using the devices and methods of the invention, one may monitor patients taking such drugs by taking frequent samples of blood and determining the number of epithelial cells in each sample as a function of time. This provides information as to the course of the disease. For example, a decreasing number of circulating epithelial cells over time suggests a decrease in the severity of the disease and the size of the tumor or tumors. Immediately following quantification of epithelial cells, these cells may be analyzed by PCR to determine what mutations may be present in the EFGR gene expressed in the epithelial cells. Certain mutations, such as those clustered around the ATP-binding pocket of the EGFR TK domain, are known to make the cancer cells susceptible to gefitinib inhibition. Thus, the presence of these mutations supports a diagnosis of cancer that is likely to respond to treatment using gefitinib. However, many patients who respond to gefitinib eventually develop a second mutation, often a methionine-to-threonine substitution at position 790 in exon 20 of the TK domain, which renders them resistant to gefitinib. By using the devices and method of the invention, one may test for this mutation as well, providing further diagnostic information about the course of the disease and the likelihood that it will respond to gefitinib or similar compounds.

Fetal Cell Detection

The devices and methods described herein may be employed on blood samples obtained from a pregnant human, e.g., to screen a fetus for a condition or abnormality. When screening a fetus, a blood sample can be obtained from a pregnant mammal or pregnant human within 24, 20, 16, 12, 10, 8, or 4 weeks of gestation. In other embodiments, screening and detection of fetal cells can occur after pregnancy has been terminated.

For example, in some embodiments, rare cells are detected by staining for antigens such as ε/γ globin (cytoplasmic), GPA, i-antigen, CD71, or a combination thereof. A combination of ε and γ globins is found on 95-100% of fetal nucleated red blood cells (fNRBC's) from 10-24 weeks gestation. Al-Mufti et al., (2001) Haematologica 85, 357-362; Choolani et al., (2003) Mol. Hum. Reprod., 9, 227-235. This ε−γ combination, or γ globin alone, has been used to stain fNRBC, e.g., as described in Bohmer, (1998) Br J Haematol. 103, 351-60; Choolani et al., (2003); Christensen et al., (2005) Fetal Diagn. Ther. 20, 106-112; and Hennerbichler et al., (2002) Cytometry, 48, 87-92. Less than 10 false positives were seen per fNRBC, with or without CD71 enrichment, thus making the globins a highly specific (>10,000 fold) triage. Antibodies to both globins are known to those skilled in the art. Staining can result in a binary score such as positive or negative or in various intensities indicating an amount of antigen in the analyte.

Glycophorin A and CD71 are additional antigens that may be used for detection of cell types. GPA is present throughout the red blood cell lineage. Thus, it can be used for identifying nucleated red blood cells, regardless of their level of maturation. GPA is thought to be found exclusively on erythroid lineage cells, and is generally found on very few circulating cells, and its presence increases during pregnancy. FACS sorting has shown a combination of CD71 and GPA to be present on at least 0.15% of mononucleated cells during pregnancy, e.g., Price et al., (1991) Am. J. Obstet Gynecol., 165, 1713-1717; Sohda et al., (1997) Prenat. Diagn., 17, 743-752.

Antigen-i can also be used as a marker for isolation and/or detection of fetal cells, e.g., Sitar et al., (2005) Exp. Cell. Res., 302, 153-161. The i-antigens were first described in the 1950s using patient polyclonal sera. Subsequent data demonstrated that the two forms of the antigen, “I” or “i”, were expressed on adult and fetal cells respectively.

Once fetal cells or components of interest are detected, they can be further analyzed for various purposes, e.g., sex or genetic condition. In some embodiments, analysis of fetal cells or components thereof is used to determine the presence or absence of a genetic abnormality, such as a chromosomal, DNA, or RNA abnormality. Examples of autosomal chromosome abnormalities include, but are not limited to, Angleman syndrome (15q11.2-q13), cri-du-chat syndrome (5p-), DiGeorge syndrome and Velo-cardiofacial syndrome (22q11.2), Miller-Dieker syndrome (17p13.3), Prader-Willi syndrome (15q11.2-q13), retinoblastoma (13q14), Smith-Magenis syndrome (17p11.2), trisomy 13, trisomy 16, trisomy 18, trisomy 21 (Down syndrome), triploidy, Williams syndrome (7q11.23), and Wolf-Hirschhorn (4p-). Examples of sex chromosome abnormalities include, but are not limited to, Kallman syndrome (Xp22.3), steroid sulfate deficiency (STS) (Xp22.3), X-linked ichthiosis (Xp22.3), Klinefelter syndrome (XXY); fragile X syndrome; Turner syndrome; metafemales or trisomy X; and monosomy X.

Other less common chromosomal abnormalities that can be analyzed by the systems herein include, but are not limited to, deletions (small missing sections); microdeletions (a minute amount of missing material that may include only a single gene); translocations (a section of a chromosome is attached to another chromosome); and inversions (a section of chromosome is snipped out and reinserted upside down).

In some embodiments, analysis of fetal cells or components thereof is used to analyze SNPs and predict a condition of the fetus based on such SNPs.

In any of the embodiments herein, detection/analysis can be made using any means known in the art. Examples of methods for detecting/analyzing genetic conditions include, but are not limited to, karyotyping, in situ hybridization (ISH) (e.g., florescence in situ hybridization (FISH), chromogenic in situ hybridization (CISH), nanogold in situ hybridization (NISH)), restriction fragment length polymorphism (RFLP) analysis, polymerase chain reaction (PCR) techniques, flow cytometry, electron microscopy, quantum dots, and nucleic acid arrays for detection of single nucleotide polymorphisms (SNPs) or levels of RNA. In some embodiments, two or more methods for detecting genetic abnormalities are performed. For example, multiple FISH probes or other DNA probes may be used in analyzing a single cell or component of interest.

Sample Preparation

Samples may be employed in the methods described herein with or without manipulation, e.g., stabilization and removal of certain components. In one embodiment, the sample is enriched in the analytes, e.g., cells, of interest prior to introduction to a device of the invention. Methods for enriching cell populations are described herein and known in the art, e.g., affinity mechanisms, magnetic properties, agglutination, and size, shape, and deformability based enrichments. Some samples may be diluted or concentrated prior to introduction into the device.

Preferably, a sample, e.g., of blood, is applied to the system herein within 1 week, 6 day, 5 days, 4 days, 3 days, 2 days, 1 day, 12 hrs, 6 hrs, 3 hrs, 2 hrs, or 1 hr from when the sample is obtained. In some embodiments, a blood sample is applied to a system herein upon withdrawal from a patient. Preferably, the sample is applied to the systems herein at a temperature of 4-37° C.

In one embodiment, reagents are added to the sample, to selectively or nonselectively increase the hydrodynamic size of the analytes within the sample. This modified sample is, for example, then pumped through a deterministic device. Because the particles are swollen and have an increased hydrodynamic size, it will be possible to use deterministic devices with larger and more easily manufactured gap sizes. In a preferred embodiment, the steps of swelling and size-based enrichment are performed in an integrated fashion on a deterministic device. Suitable reagents include any hypotonic solution, e.g., deionized water, 2% sugar solution, or neat non-aqueous solvents. Other reagents include beads, e.g., magnetic or polymer, that bind selectively (e.g., through antibodies or avidin-biotin) or non-selectively.

In another embodiment, reagents are added to the sample to selectively or nonselectively decrease the hydrodynamic size of the particles within the sample. Nonuniform decrease in particles in a sample will increase the difference in hydrodynamic size between particles. For example, nucleated cells are separated from enucleated cells by hypertonically shrinking the cells. The enucleated cells can shrink to a very small particle, while the nucleated cells cannot shrink below the size of the nucleus. Exemplary shrinking reagents include hypertonic solutions.

In an alternative embodiment, affinity functionalized beads are used to increase the hydrodynamic size of an analyte of interest relative to other analytes present in a sample, thereby allowing for the operation of a deterministic device with a larger and more easily manufactured gap size.

Such alterations of size may be employed in series or in parallel with magnetic based enrichment, as described herein.

When a sample, e.g., of blood, is obtained it may be collected in a container including one or more of the following agents: a stabilizing agent, a preservative, a fixant, a lysing agent, a diluent, an anti-apoptotic agent, an anti-coagulation agent, an anti-thrombotic agent, a buffering agent, an osmolality regulating agent, a pH regulating agent, a reagent that alters a magnetic property, and/or a cross-linking agent.

Fluids may be driven through a device either actively or passively. Fluids may be pumped using electric field, a centrifugal field, pressure-driven fluid flow, an electro-osmotic flow, or capillary action. In preferred embodiments, the average direction of the field will be parallel to the walls of the channel.

Any of the following exemplary deterministic devices and methods may be incorporated into devices of the invention.

### EXAMPLES

### Example 1

### A Silicon Device Multiplexing 14 3-Stage Array Duplexes

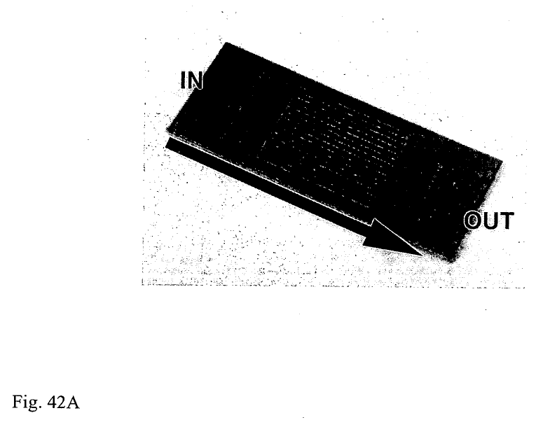

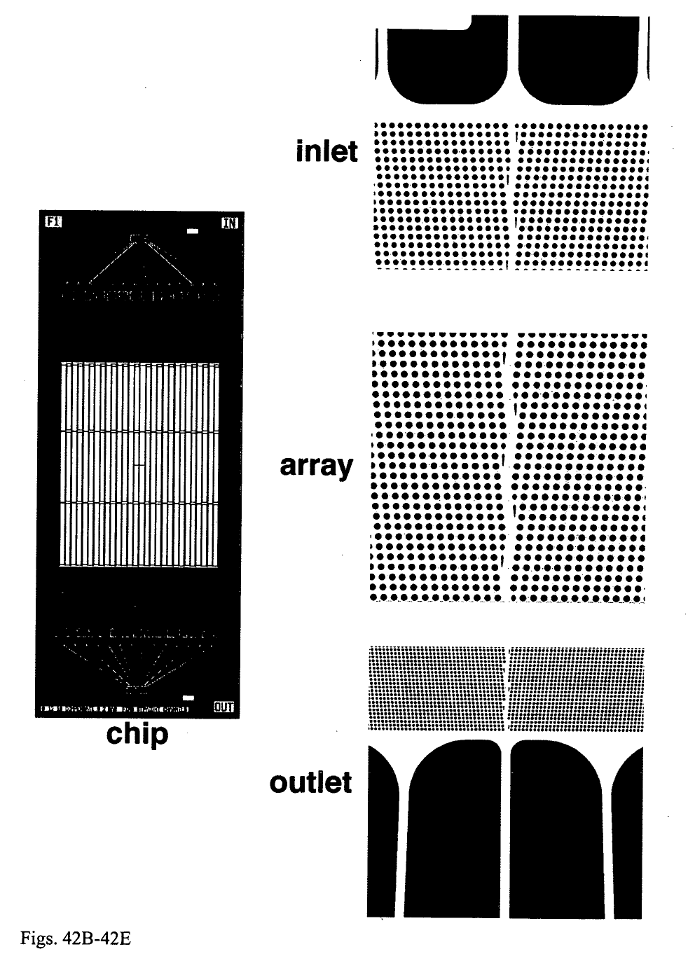

FIGS. 42A-42E show an exemplary device, characterized as follows.

Dimension: 90 mm×34 mm×1 mm

Array design: 3 stages, gap size=18, 12 and 8 μm for the first, second and third stage, respectively. Bifurcation ratio=1/10. Duplex; single bypass channel

Device design: multiplexing 14 array duplexes; flow resistors for flow stability

Device fabrication: The arrays and channels were fabricated in silicon using standard photolithography and deep silicon reactive etching techniques. The etch depth is 150 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device Packaging: The device was mechanically mated to a plastic manifold with external fluidic reservoirs to deliver blood and buffer to the device and extract the generated fractions.

Device Operation: An external pressure source was used to apply a pressure of 2.4 PSI to the buffer and blood reservoirs to modulate fluidic delivery and extraction from the packaged device.

Experimental conditions: human blood from consenting adult donors was collected into K₂EDTA vacutainers (366643, Becton Dickinson, Franklin Lakes, N.J.). The undiluted blood was processed using the exemplary device described above (FIG. 42F) at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.).

Measurement techniques: Complete blood counts were determined using a Coulter impedance hematology analyzer (COULTER® Ac-T diff™, Beckman Coulter, Fullerton, Calif.).

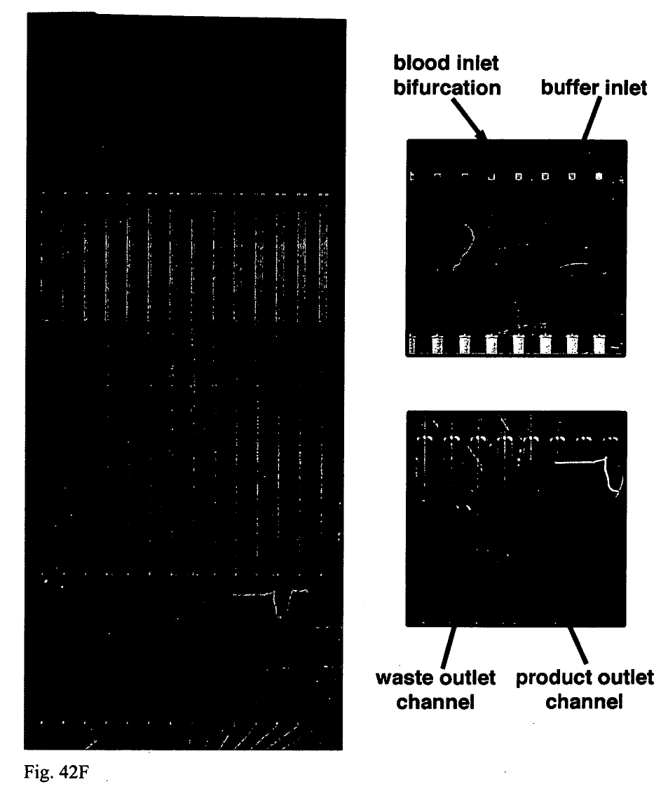

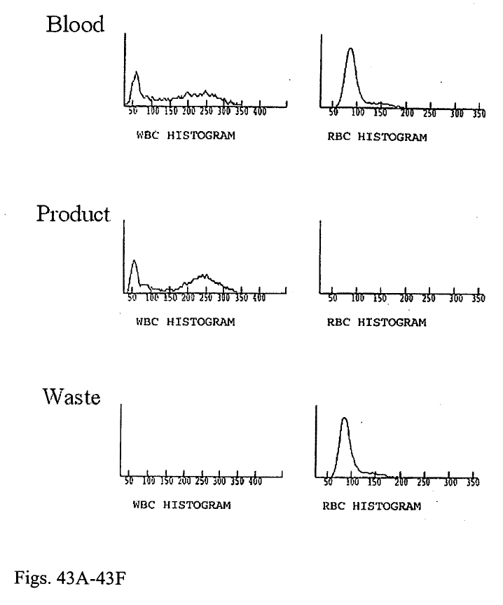

Performance: FIGS. 43A-43F shows typical histograms generated by the hematology analyzer from a blood sample and the waste (buffer, plasma, red blood cells, and platelets) and product (buffer and nucleated cells) fractions generated by the device. Table 1 shows the performance over 5 different blood samples:

TABLE 1

| | | Performance Metrics | | |
|---|---|---|---|---|
| Sample | | RBC | Platelet | WBC |
| number | Throughput | removal | removal | loss |
| 1 | 4 mL/hr | 100% | 99% | <1% |
| 2 | 6 mL/hr | 100% | 99% | <1% |
| 3 | 6 mL/hr | 100% | 99% | <1% |
| 4 | 6 mL/hr | 100% | 97% | <1% |
| 5 | 6 mL/hr | 100% | 98% | <1% |

### Example 2

### A Silicon Device Multiplexing 14 Single-Stage Array Duplexes

FIGS. 44A-44D show an exemplary device, characterized as follows.

Dimension: 90 mm×34 mm×1 mm

Array design: 1 stage, gap size=24 μm. Bifurcation ratio=1/60. Duplex; double bypass channel

Device design: multiplexing 14 array duplexes; flow resistors for flow stability Device fabrication: The arrays and channels were fabricated in silicon using standard photolithography and deep silicon reactive etching techniques. The etch depth is 150 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device Packaging: The device was mechanically mated to a plastic manifold with external fluidic reservoirs to deliver blood and buffer to the device and extract the generated fractions.

Device Operation: An external pressure source was used to apply a pressure of 2.4 PSI to the buffer and blood reservoirs to modulate fluidic delivery and extraction from the packaged device.

Experimental conditions: human blood from consenting adult donors was collected into K₂EDTA vacutainers (366643, Becton Dickinson, Franklin Lakes, N.J.). The undiluted blood was processed using the exemplary device described above at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.).

Measurement techniques: Complete blood counts were determined using a Coulter impedance hematology analyzer (COULTER® Ac-T diff™, Beckman Coulter, Fullerton, Calif.).

Performance: The device operated at 17 μL/hr and achieved >99% red blood cell removal, >95% nucleated cell retention, and >98% platelet removal.

### Example 3

### Separation of Fetal Cord Blood

FIG. 45 shows a schematic of the device used to separate nucleated cells from fetal cord blood.

Dimension: 100 mm×28 mm×1 mm

Array design: 3 stages, gap size=18, 12 and 8 μm for the first, second and third stage, respectively. Bifurcation ratio=1/10. Duplex; single bypass channel.

Device design: multiplexing 10 array duplexes; flow resistors for flow stability.

Device fabrication: The arrays and channels were fabricated in silicon using standard photolithography and deep silicon reactive etching techniques. The etch depth is 140 μm. Through holes for fluid access are made using KOH wet etching. The silicon substrate was sealed on the etched face to form enclosed fluidic channels using a blood compatible pressure sensitive adhesive (9795, 3M, St Paul, Minn.).

Device Packaging: The device was mechanically mated to a plastic manifold with external fluidic reservoirs to deliver blood and buffer to the device and extract the generated fractions.

Device Operation: An external pressure source was used to apply a pressure of 2.0 PSI to the buffer and blood reservoirs to modulate fluidic delivery and extraction from the packaged device.

Experimental conditions: Human fetal cord blood was drawn into phosphate buffered saline containing Acid Citrate Dextrose anticoagulants. 1 mL of blood was processed at 3 mL/hr using the device described above at room temperature and within 48 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.) and 2 mM EDTA (15575-020, Invitrogen, Carlsbad, Calif.).

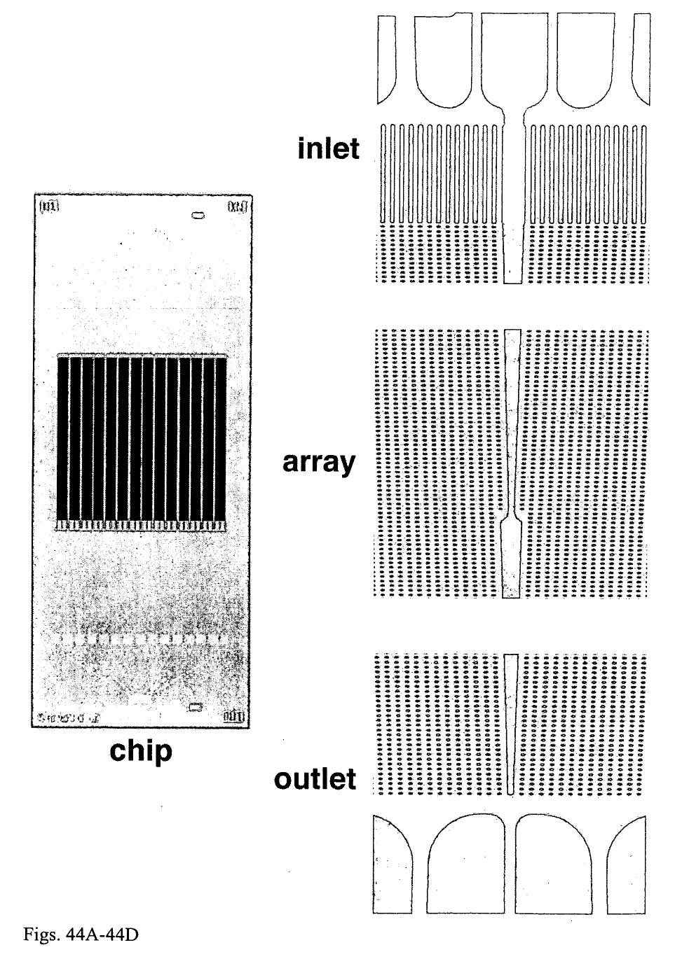

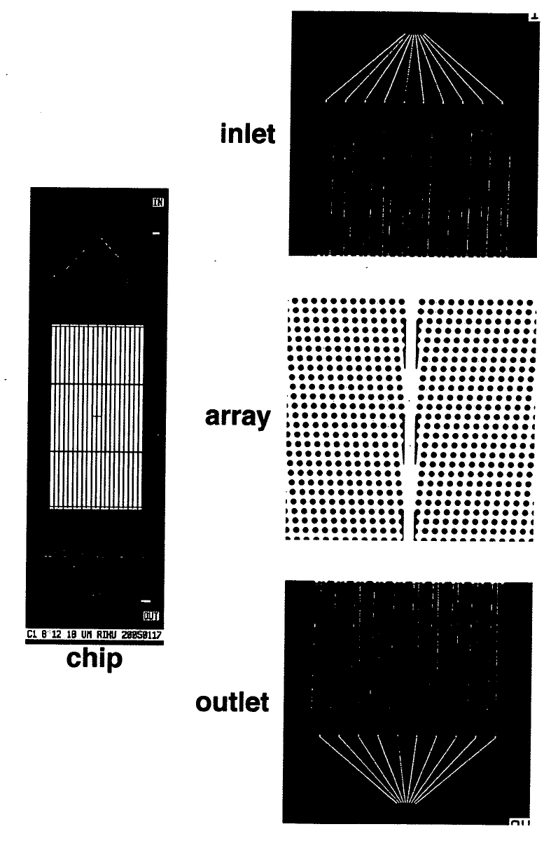

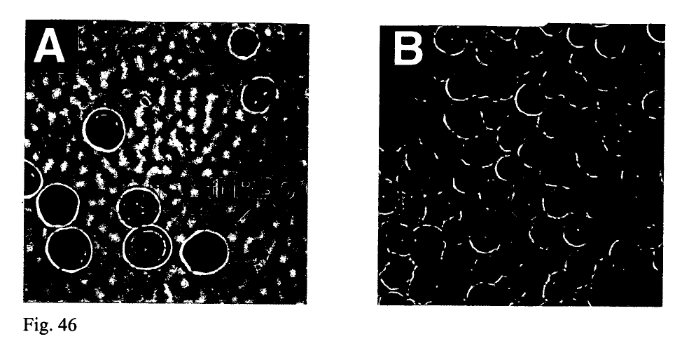

Measurement techniques: Cell smears of the product and waste fractions (FIG. 46A-46B) were prepared and stained with modified Wright-Giemsa (WG16, Sigma Aldrich, St. Louis, Mo.).

Performance: Fetal nucleated red blood cells were observed in the product fraction (FIG. 46A) and absent from the waste fraction (FIG. 46B).

### Example 4

### Isolation of Fetal Cells from Maternal Blood

The device and process described in detail in Example 1 were used in combination with immunomagnetic affinity enrichment techniques to demonstrate the feasibility of isolating fetal cells from maternal blood.

Experimental conditions: blood from consenting maternal donors carrying male fetuses was collected into K₂EDTA vacutainers (366643, Becton Dickinson, Franklin Lakes, N.J.) immediately following elective termination of pregnancy. The undiluted blood was processed using the device described in Example 1 at room temperature and within 9 hrs of draw. Nucleated cells from the blood were separated from enucleated cells (red blood cells and platelets), and plasma delivered into a buffer stream of calcium and magnesium-free Dulbecco's Phosphate Buffered Saline (14190-144, Invitrogen, Carlsbad, Calif.) containing 1% Bovine Serum Albumin (BSA) (A8412-100ML, Sigma-Aldrich, St Louis, Mo.). Subsequently, the nucleated cell fraction was labeled with anti-CD71 microbeads (130-046-201, Miltenyi Biotech Inc., Auburn, Calif.) and enriched using the MiniMACS™ MS column (130-042-201, Miltenyi Biotech Inc., Auburn, Calif.) according to the manufacturer's specifications. Finally, the CD71-positive fraction was spotted onto glass slides.

Measurement techniques: Spotted slides were stained using fluorescence in situ hybridization (FISH) techniques according to the manufacturer's specifications using Vysis probes (Abbott Laboratories, Downer's Grove, Ill.). Samples were stained from the presence of X and Y chromosomes. In one case, a sample prepared from a known trisomy 21 pregnancy was also stained for chromosome 21.

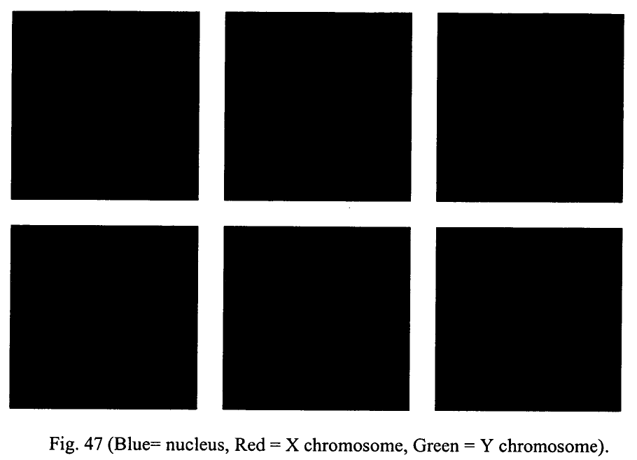

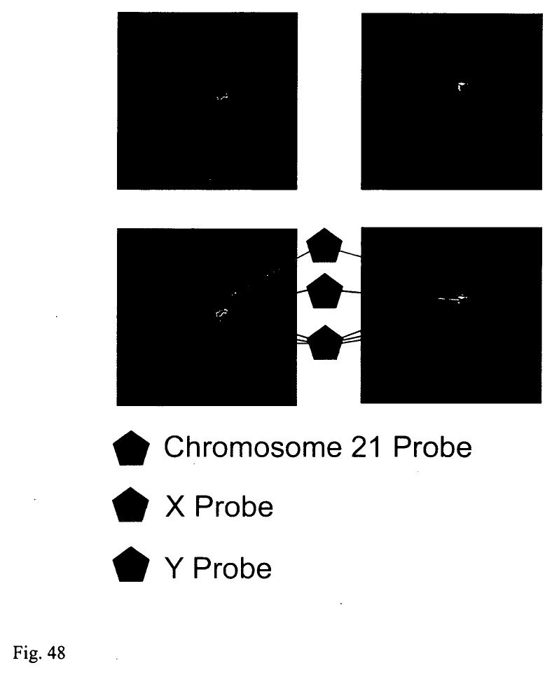

Performance: Isolation of fetal cells was confirmed by the reliable presence of male cells in the CD71-positive population prepared from the nucleated cell fractions (FIG. 47). In the single abnormal case tested, the trisomy 21 pathology was also identified (FIG. 48).

The following examples show specific embodiments of devices. The description for each device provides the number of stages in series, the gap size for each stage, e (Flow Angle), and the number of channels per device (Arrays/Chip). Each device was fabricated out of silicon using DRIE, and each device had a thermal oxide layer.

### Example 5

This device includes five stages in a single array.

Array Design: 5 stage, asymmetric array

Gap Sizes: Stage 1: 8 μm

Stage 2: 10 μm

Stage 3: 12 μm

Stage 4: 14 μm

Stage 5: 16 μm

Flow Angle: 1/10

Arrays/Chip: 1

### Example 6

This device includes the stages, where each stage is a duplex having a bypass channel. The height of the device was 125 μm.

Array Design: symmetric 3 stage array with central collection channel

Gap Sizes: Stage 1: 8 μm

Stage 2: 12 μm

Stage 3: 18 μm

Stage 4:

Stage 5:

Flow Angle: 1/10

Arrays/Chip: 1

Other: central collection channel

FIG. 49A shows the mask employed to fabricate the device. Figures B1B-B1D are enlargements of the portions of the mask that define the inlet, array, and outlet.

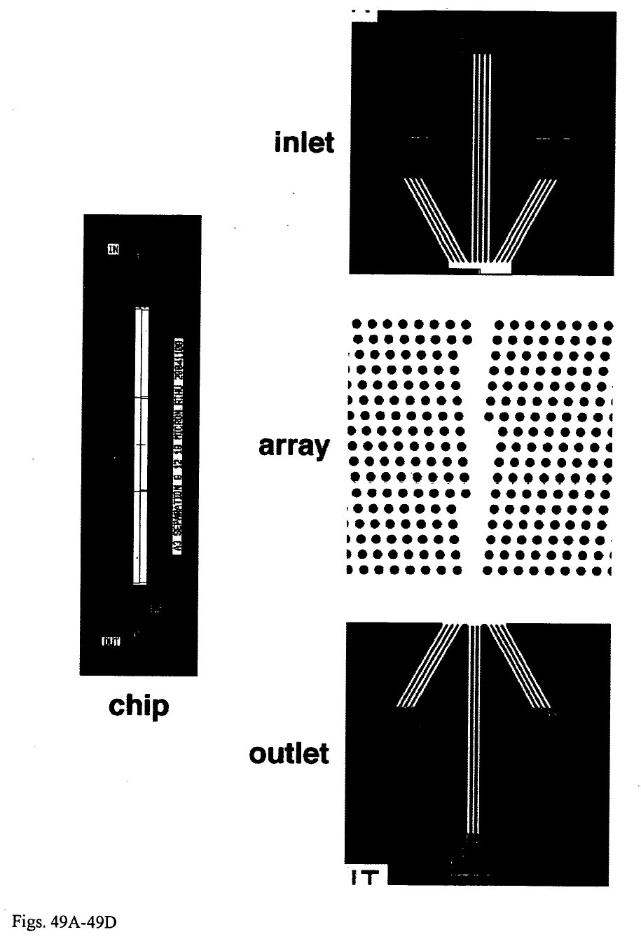

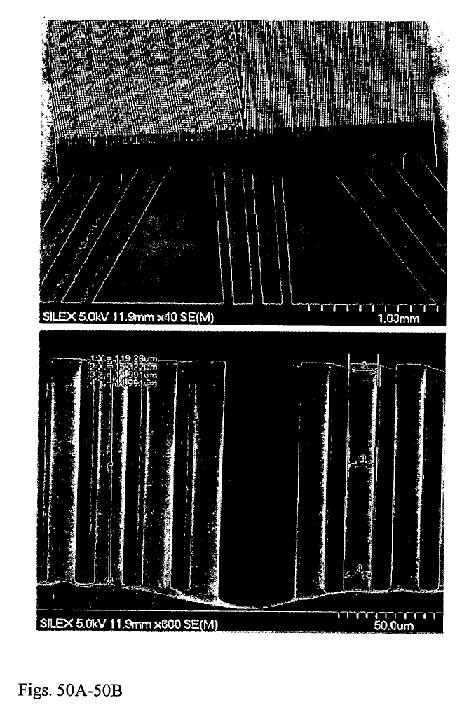

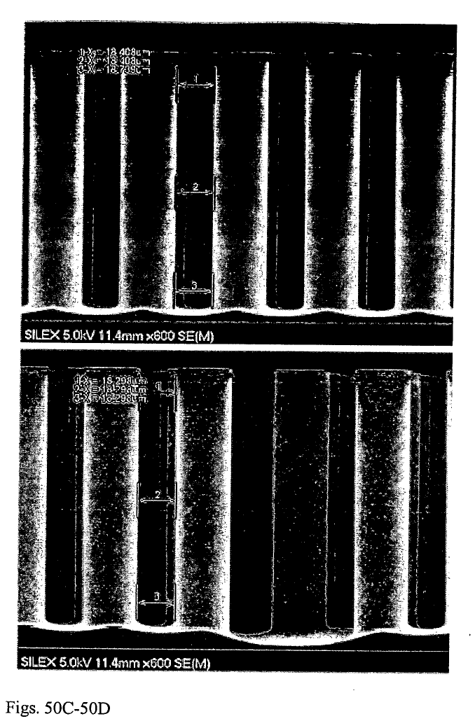

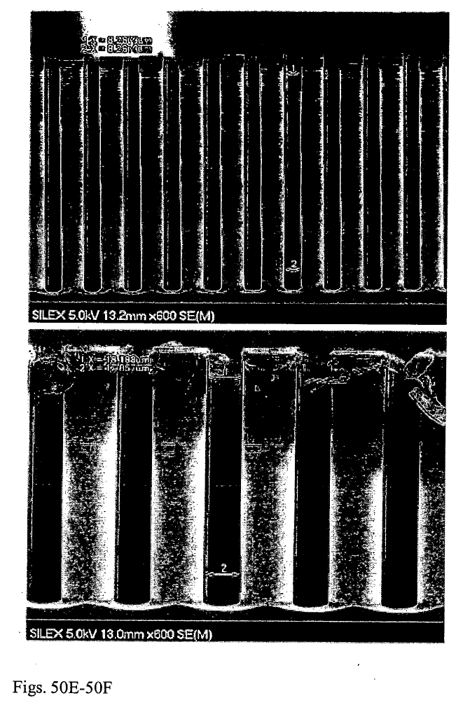

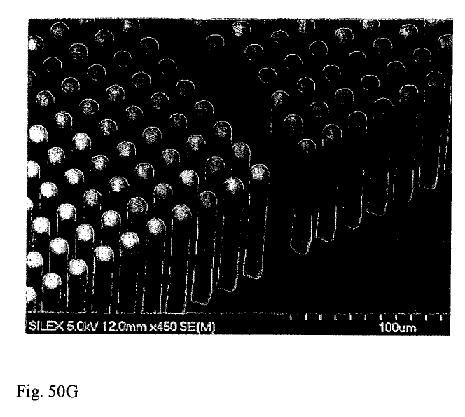

FIGS. 50A-50G show SEMs of the actual device.

### Example 7

This device includes the stages, where each stage is a duplex having a bypass channel. “Fins” were designed to flank the bypass channel to keep fluid from the bypass channel from re-entering the array. The chip also included on-chip flow resistors, i.e., the inlets and outlets possessed greater fluidic resistance than the array. The height of the device was 117 μm.

Array Design: 3 stage symmetric array

Gap Sizes: Stage 1: 8 μm

Stage 2: 12 μm

Stage 3: 18 μm

Stage 4:

Stage 5:

Flow Angle: 1/10

Arrays/Chip: 10

Other: large fin central collection channel on-chip flow resistors

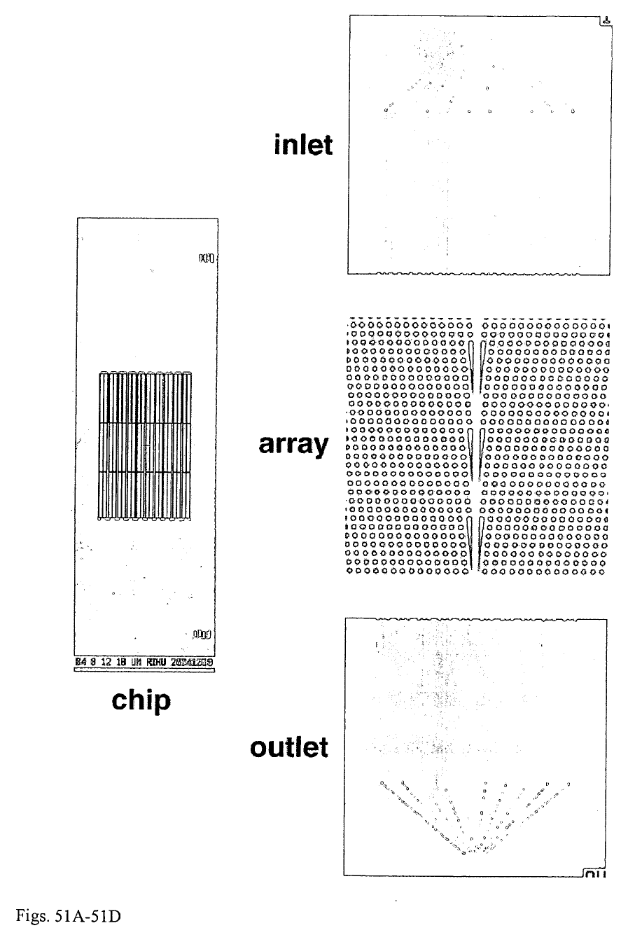

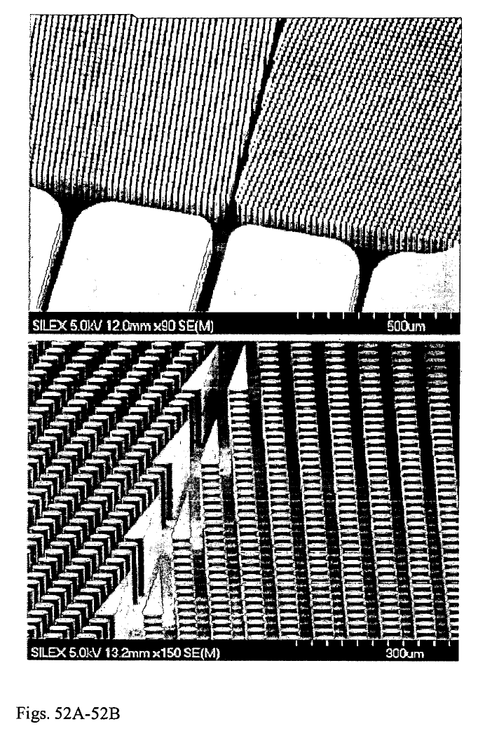

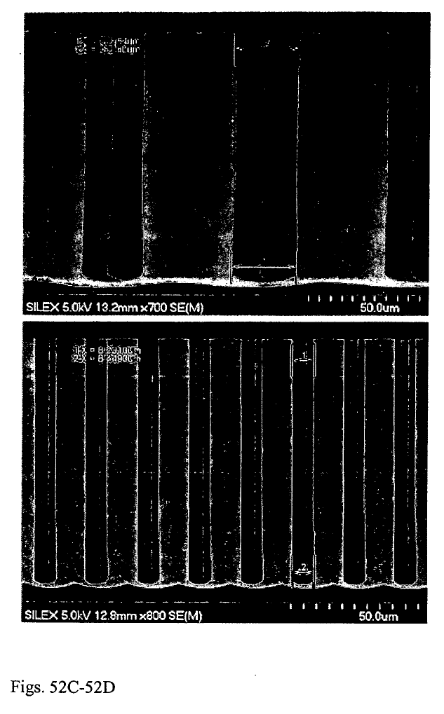

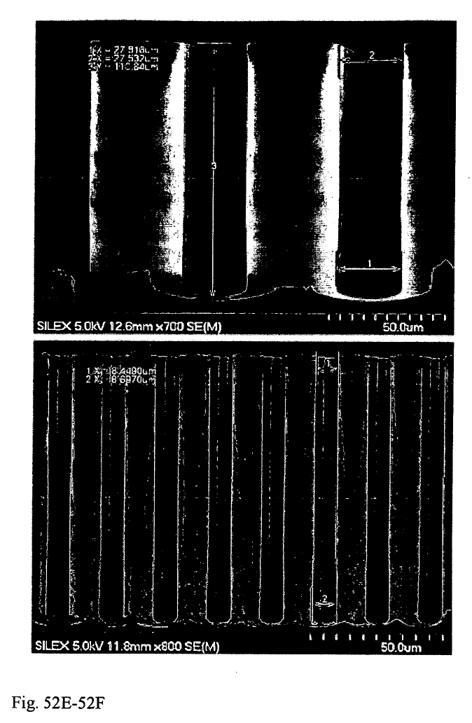

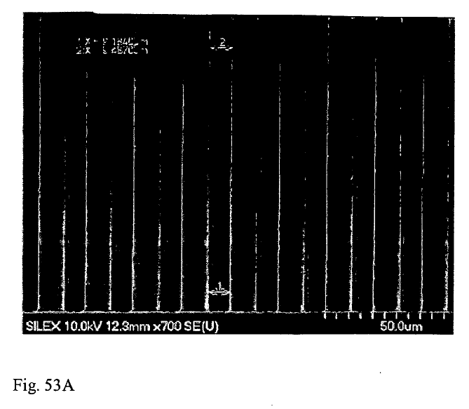

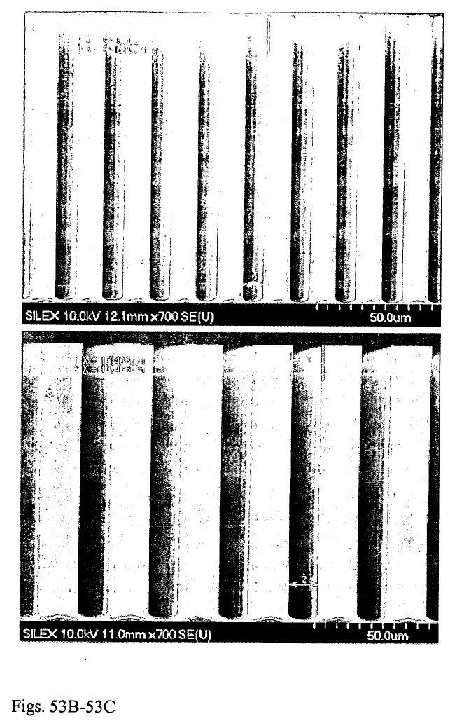

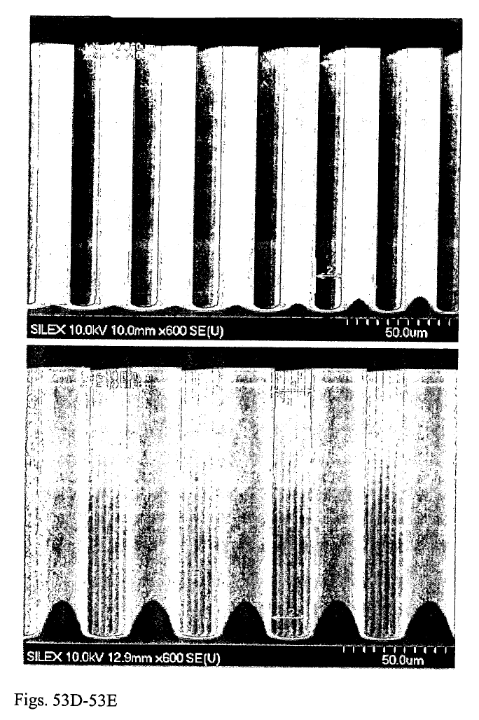

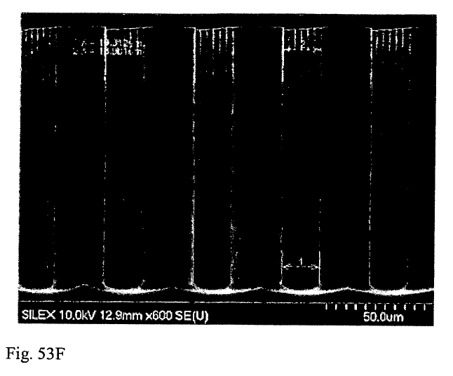

FIG. 51A shows the mask employed to fabricate the device. FIGS. 51B-51D are enlargements of the portions of the mask that define the inlet, array, and outlet. FIGS. 52A-52F show SEMs of the actual device.

### Example 8

This device includes the stages, where each stage is a duplex having a bypass channel. “Fins” were designed to flank the bypass channel to keep fluid from the bypass channel from re-entering the array. The edge of the fin closest to the array was designed to mimic the shape of the array. The chip also included on-chip flow resistors, i.e., the inlets and outlets possessed greater fluidic resistance than the array. The height of the device was 138 μm.

| | |
|---|---|
| Array Design: | 3 stage symmetric array |
| Gap Sizes: | Stage 1: 8 μm |
| | Stage 2: 12 μm |
| | Stage 3: 18 μm |
| | Stage 4: |
| | Stage 5: |
| Flow Angle: | 1/10 |
| Arrays/Chip: | 10 |
| Other: | alternate large fin central collection channel |
| | on-chip flow resistors |

FIG. 45A shows the mask employed to fabricate the device. FIGS. 45B-45D are enlargements of the portions of the mask that define the inlet, array, and outlet. FIGS. 532A-532F show SEMs of the actual device.

### Example 9

This device includes the stages, where each stage is a duplex having a bypass channel. “Fins” were optimized using Femlab to flank the bypass channel to keep fluid from the bypass channel from re-entering the array. The edge of the fin closest to the array was designed to mimic the shape of the array. The chip also included on-chip flow resistors, i.e., the inlets and outlets possessed greater fluidic resistance than the array. The height of the device was 139 or 142 μm.

Array Design: 3 stage symmetric array

Gap Sizes: Stage 1: 8 μm

Stage 2: 12 μm

Stage 3: 18 μm

Stage 4:

Stage 5:

Flow Angle: 1/10

Arrays/Chip: 10

Other: Femlab optimized central collection channel (Femlab I) on-chip flow resistors

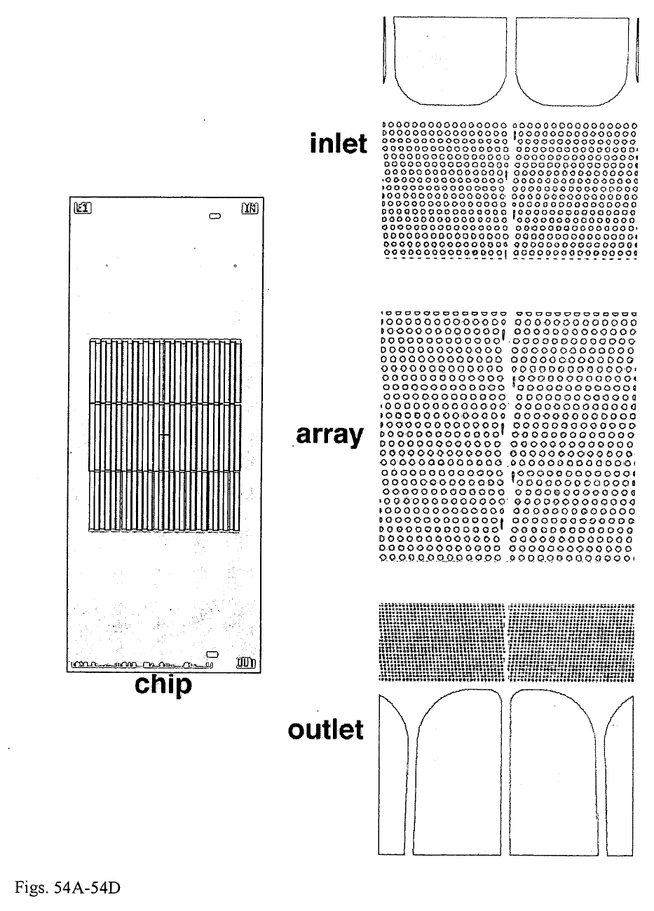

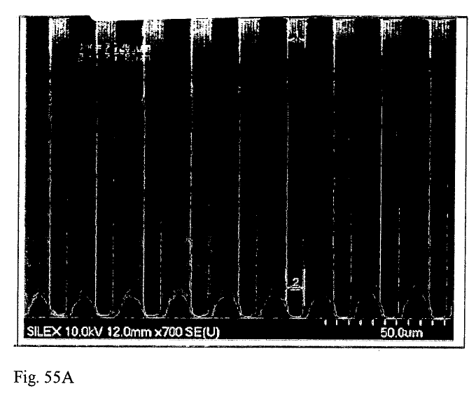

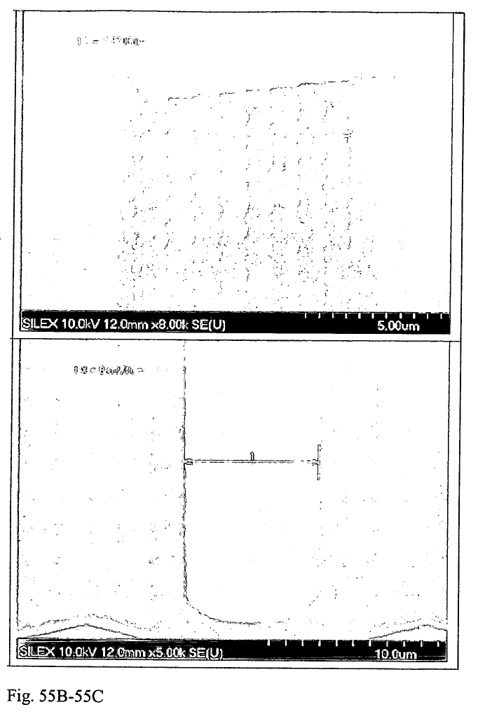

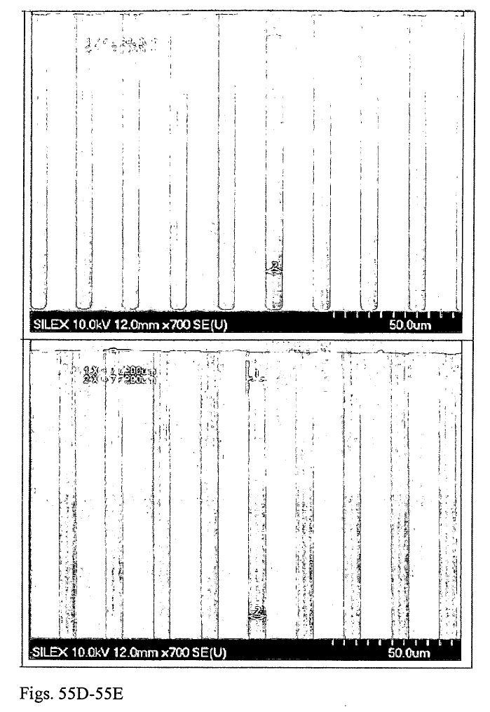

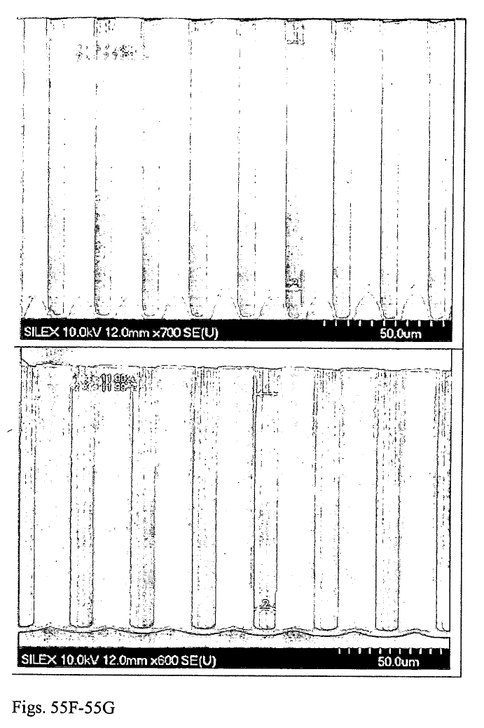

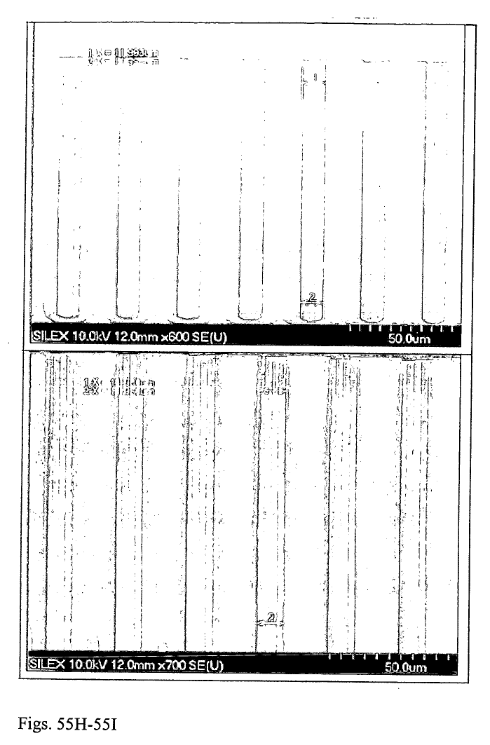

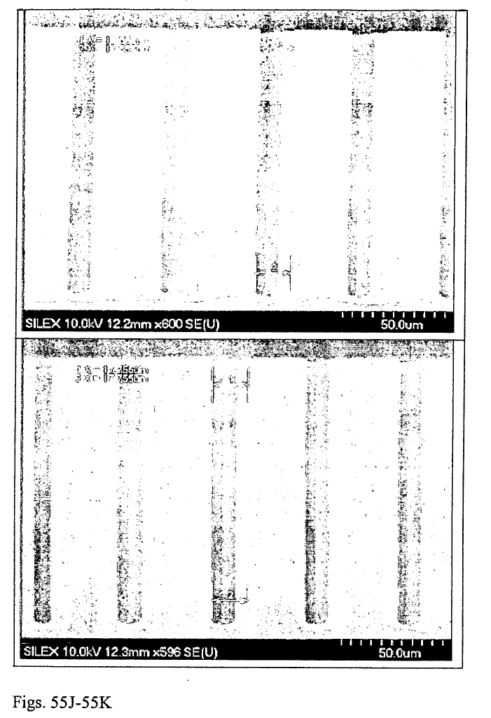

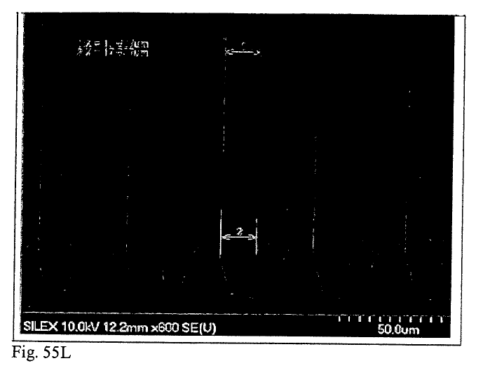

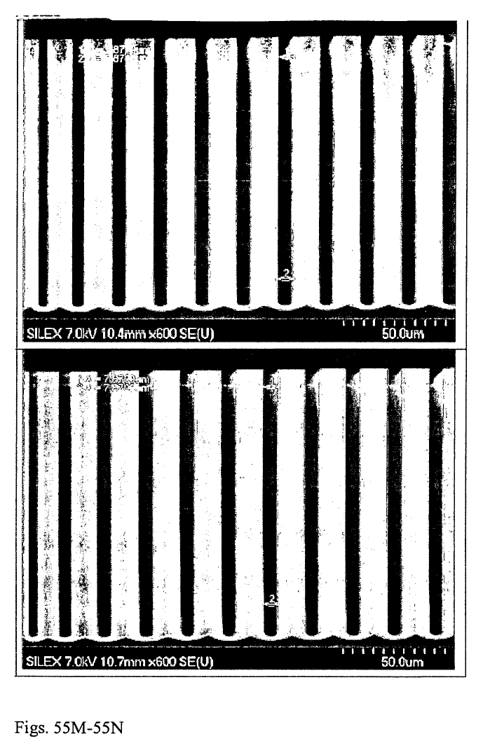

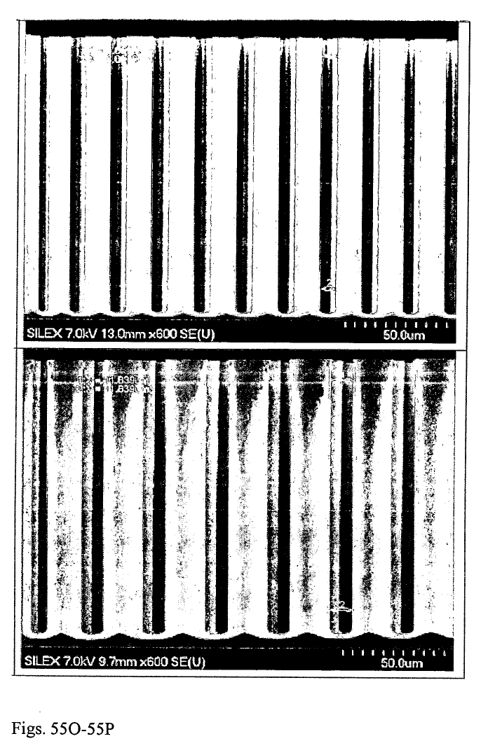

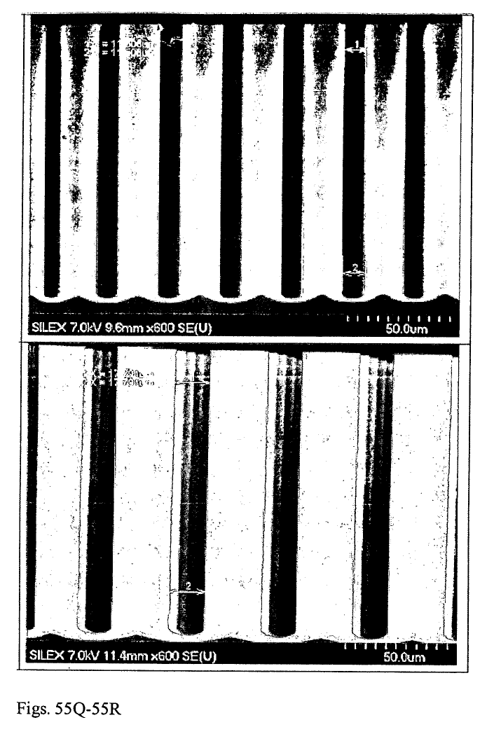

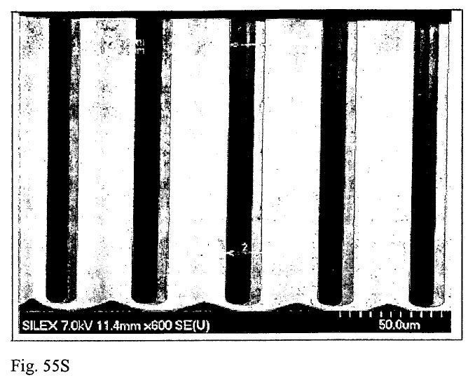

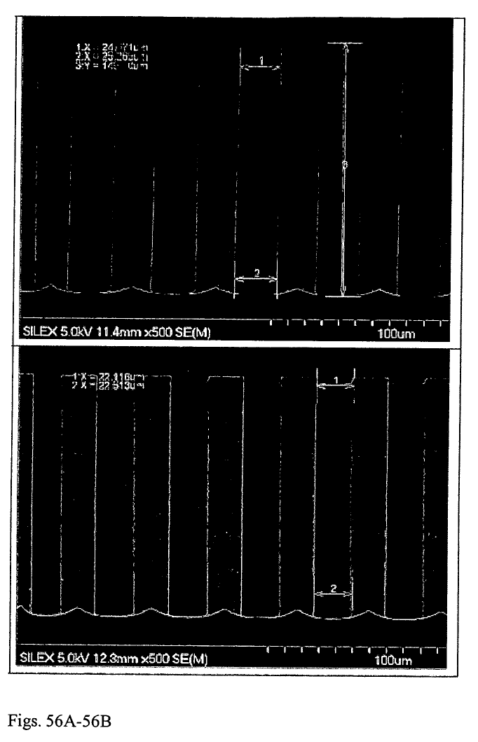

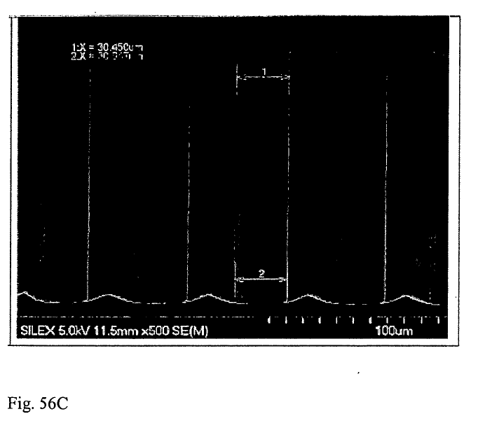

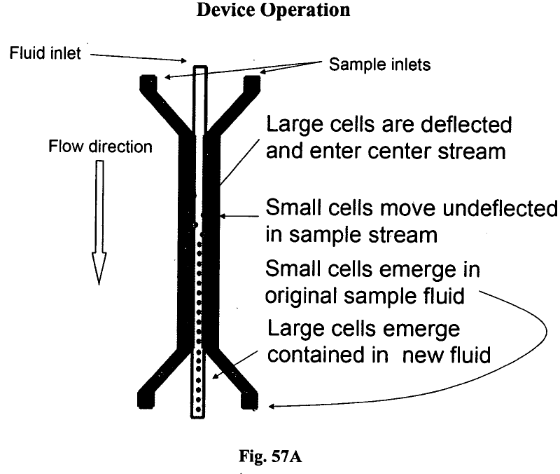

FIG. 54A shows the mask employed to fabricate the device. FIGS. 54B-E1D are enlargements of the portions of the mask that define the inlet, array, and outlet. FIGS. 55A-55S show SEMs of the actual device.

### Example 10

This device includes a single stage, duplex device having a bypass channel disposed to receive output from the ends of both arrays. The obstacles in this device are elliptical. The array boundary was modeled in Femlab to. The chip also included on-chip flow resistors, i.e., the inlets and outlets possessed greater fluidic resistance than the array. The height of the device was 152 μm.

Array Design: single stage symmetric array

Gap Sizes: Stage 1: 24 μm

Stage 2:

Stage 3:

Stage 4:

Stage 5:

Flow Angle: 1/60

Arrays/Chip: 14

Other: central barrier

ellipsoid posts

on-chip resistors

Femlab modeled array boundary

FIG. 44A shows the mask employed to fabricate the device. FIGS. 44B-44D are enlargements of the portions of the mask that define the inlet, array, and outlet. FIGS. 56A-56C show SEMs of the actual device.

### Example 11

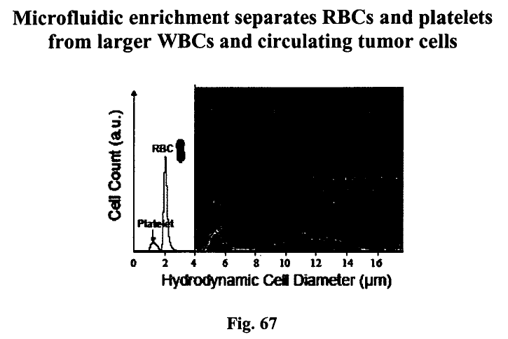

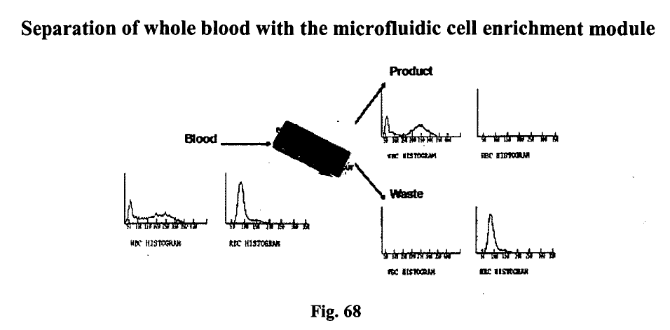

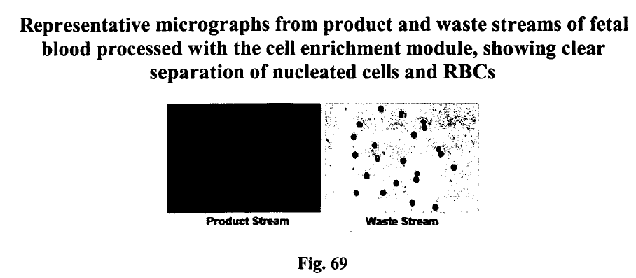

Deterministic devices incorporated into devices of the invention were designed by computer-aided design (CAD) and microfabricated by photolithography. A two-step process was developed in which a blood sample is first debulked to remove the large population of small cells, and then the rare target epithelial cells target cells are recovered by immunoaffinity capture. The devices were defined by photolithography and etched into a silicon substrate based on a CAD-generated design. The cell enrichment module, which is approximately the size of a standard microscope slide, contains 14 parallel sample processing sections and associated sample handling channels that connect to common sample and buffer inlets and product and waste outlets. Each section contains an array of microfabricated obstacles that is optimized to separate the target cell type by size via displacement of the larger cells into the product stream. In this example, the microchip was designed to separate red blood cells (RBCs) and platelets from the larger leukocytes and circulating tumor cells. Enriched populations of target cells were recovered from whole blood passed through the device. Performance of the cell enrichment microchip was evaluated by separating RBCs and platelets from white blood cells (WBCs) in normal whole blood (FIG. 67). In cancer patients, circulating tumor cells are found in the larger WBC fraction. Blood was minimally diluted (30%), and a 6 ml sample was processed at a flow rate of up to 6 ml/hr. The product and waste stream were evaluated in a Coulter Model “A^{C}-T diff” clinical blood analyzer, which automatically distinguishes, sizes, and counts different blood cell populations. The enrichment chip achieved separation of RBCs from WBCs, in which the WBC fraction had >99% retention of nucleated cells, >99% depletion of RBCs and >97% depletion of platelets. Representative histograms of these cell fractions are shown in FIG. 68. Routine cytology confirmed the high degree of enrichment of the WBC RBC fractions (FIG. 69).

Next, epithelial cells were recovered by affinity capture in a microfluidic module that is functionalized with immobilized antibody. A capture module with a single chamber containing a regular array of antibody-coated microfabricated obstacles was designed. These obstacles are disposed to maximize cell capture by increasing the capture area approximately four-fold, and by slowing the flow of cells under laminar flow adjacent to the obstacles to increase the contact time between the cells and the immobilized antibody. The capture modules can be operated under conditions of relatively high flow rate but low shear to protect cells against damage. The surface of the capture module was functionalized by sequential treatment with 10% silane, 0.5% gluteraldehyde and avidin, followed by biotinylated anti-EpCAM. Active sites were blocked with 3% bovine serum albumin in PBS, quenched with dilute Tris HCl and stabilized with dilute L-histidine. Modules were washed in PBS after each stage and finally dried and stored at room temperature. Capture performance was measured with the human advanced lung cancer cell line NCI-H1650 (ATCC Number CRL-5883). This cell line has a heterozygous 15 bp in-frame deletion in exon 19 of EGFR that renders it susceptible to gefitinib. Cells from confluent cultures were harvested with trypsin, stained with the vital dye Cell Tracker Orange (CMRA reagent, Molecular Probes, Eugene, Oreg.), resuspended in fresh whole blood and fractionated in the microfluidic chip at various flow rates. In these initial feasibility experiments, cell suspensions were processed directly in the capture modules without prior fractionation in the cell enrichment module to debulk the red blood cells; hence, the sample stream contained normal blood red cells and leukocytes as well as tumor cells. After the cells were processed in the capture module, the device was washed with buffer at a higher flow rate (3 ml/hr) to remove the nonspecifically bound cells. The adhesive top was removed and the adherent cells were fixed on the chip with paraformaldehyde and observed by fluorescence microscopy. Cell recovery was calculated from hemacytometer counts; representative capture results are shown in Table 2. Initial yields in reconstitution studies with unfractionated blood were greater than 60% with less than 5% of non-specific binding.

TABLE 2

| Run | Avg. flow | Length of | No. cells | No. cells | |
|---|---|---|---|---|---|
| number | rate | run | processed | captured | Yield |
| 1 | 3.0 | 1 hr | 150,000 | 38,012 | 25% |
| 2 | 1.5 | 2 hr | 150,000 | 30,000/ml | 60% |
| 3 | 1.08 | 2 hr | 108,000 | 68,661 | 64% |
| 4 | 1.21 | 2 hr | 121,000 | 75,491 | 62% |

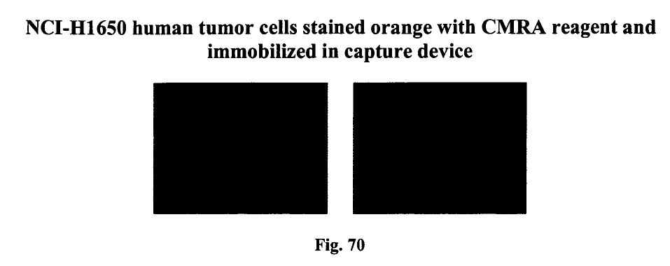

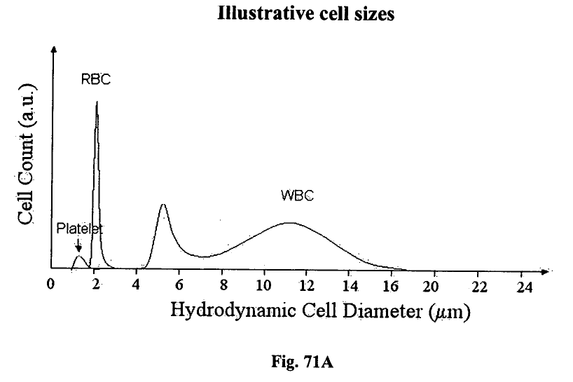

Next, NCI-H1650 cells that were spiked into whole blood and recovered by size fractionation and affinity capture as described above were successfully analyzed in situ. In a trial run to distinguish epithelial cells from leukocytes, 0.5 ml of a stock solution of fluorescein-labeled CD45 pan-leukocyte monoclonal antibody was passed into the capture module and incubated at room temperature for 30 minutes. The module was washed with buffer to remove unbound antibody and the cells were fixed on the chip with 1% paraformaldehyde and observed by fluorescence microscopy. As shown in FIG. 70, the epithelial cells were bound to the obstacles and floor of the capture module. Background staining of the flow passages with CD45 pan-leukocyte antibody is visible, as are several stained leukocytes, apparently due to a low level of non-specific capture.

### Example 12

### Device Embodiments

A design for preferred deterministic device is shown in FIG. 73A, and parameters corresponding to three preferred device embodiments associated with this design are shown in FIG. 73B. These embodiments are particularly useful for separating epithelial cells from blood.

### Example 13

### PCR Assay for EGFR Mutations

A blood sample from a cancer patient is processed and analyzed using the devices and methods of Example 11, resulting in an enriched sample of epithelial cells containing CTCs. This sample is then analyzed to identify potential EGFR mutations.

To perform this analysis, genomic DNA is isolated from the target cells present in the enriched sample and amplified for use in allele-specific Real Time PCR assays. Since all EGFR mutations in NSC lung cancer reported to date that are known to confer sensitivity or resistance to gefitinib lie within the coding regions of exons 18 to 21, each of these four exons is PCR-amplified with a unique set of exon-specific primers. Next, multiplexed allele-specific quantitative PCR reactions are performed using the TaqMan 5′ nuclease assay PCR system (Applied Biosystems) and a model 7300 Applied Biosystems Real Time PCR machine. This allows the rapid identification of any of the known clinically relevant mutations.

A two-step PCR protocol is required for this method. First, exons 18 through 21 are amplified in standard PCR reactions. The resultant PCR products are split into separate aliquots for use in allele-specific multiplexed Real Time PCR assays. The initial PCR reactions are stopped during the log phase in order to minimize possible loss of allele-specific information during amplification. Next, a second round of PCR amplifies subregions of the initial PCR product specific to each mutation of interest. Given the very high sensitivity of Real Time PCR, it is possible to obtain complete information on the mutation status of the EGFR gene even if as few as 10 CTCs are isolated. Real Time PCR provides quantification of allelic sequences over 8 logs of input DNA concentrations; thus, even heterozygous mutations in impure populations are easily detected using this method.

Oligonucleotides are designed using the primer optimization software program Primer Express (Applied Biosystems), and hybridization conditions are optimized to distinguish the wild type EGFR DNA sequence from mutant alleles. EGFR genomic DNA amplified from lung cancer cell lines that are known to carry EGFR mutations, such as H358 (wild type), H1650 (15-bp deletion, A2235-2249), and H1975 (two point mutations, 2369 C→T, 2573 T→G), is used to optimize the allele-specific Real Time PCR reactions. Using the TaqMan 5′ nuclease assay, allele-specific labeled probes specific for wild type sequence or for known EGFR mutations are developed. The oligonucleotides are designed to have melting temperatures that easily distinguish a match from a mismatch, and the Real Time PCR conditions are optimized to distinguish wild type and mutant alleles. All Real Time PCR reactions are carried out in triplicate.

Initially, labeled probes containing wild type sequence are multiplexed in the same reaction with a single mutant probe. Expressing the results as a ratio of one mutant allele sequence versus wild type sequence can identify samples containing or lacking a given mutation. After conditions are optimized for a given probe set, it is then possible to multiplex probes for all of the mutant alleles within a given exon within the same Real Time PCR assay, increasing the ease of use of this analytical tool in clinical settings.

The purity of the input sample of CTCs may vary, and the mutation status of the isolated CTCs may be heterogeneous. Nevertheless, the extremely high sensitivity of Real Time PCR enables the identification any and all mutant sequences present.

### Example 14

### Determining Counts for Non-Epithelial Cell Types

Using the methods of the invention, one may make a diagnosis based on counting cell types other than epithelial cells. A diagnosis of the absence, presence, or progression of cancer may be based on the number of cells in a cellular sample that are larger than a particular cutoff size. For example, cells with a hydrodynamic cell diameter of 14 microns or larger may be selected. This cutoff size would eliminate most leukocytes. The nature of these cells may then be determined by downstream molecular or cytological analysis.

Cell types other than epithelial cells that would be useful to analyze include endothelial cells, endothelial progenitor cells, endometrial cells, or trophoblasts indicative of a disease state. Furthermore, determining separate counts for epithelial cells and other cell types, followed by a determination of the ratios between the number of epithelial cells and the number of other cell types, may provide useful diagnostic information.

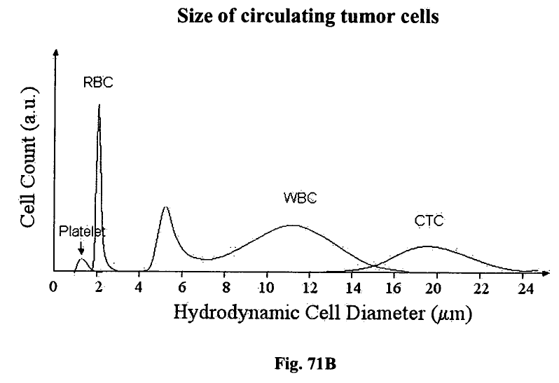

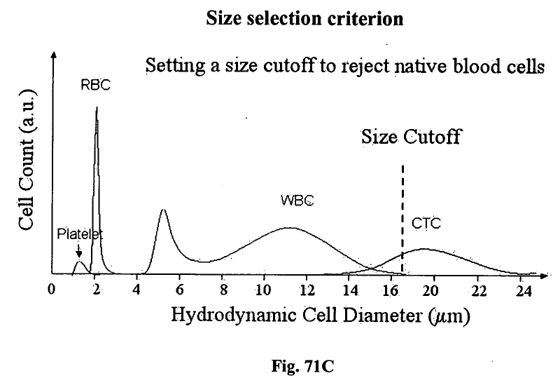

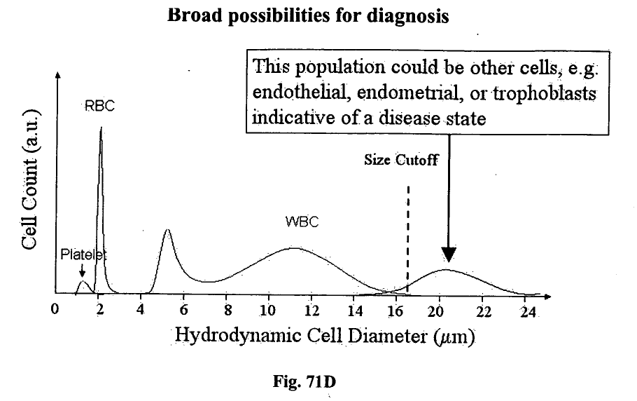

A deterministic device may be configured to isolate targeted subpopulations of cells such as those described above, as shown in FIG. 71A-D. A size cutoff may be selected such that most native blood cells, including red blood cells, white blood cells, and platelets, flow to waste, while non-native cells, which could include endothelial cells, endothelial progenitor cells, endometrial cells, or trophoblasts, are collected in an enriched sample. This enriched sample may be further analyzed.

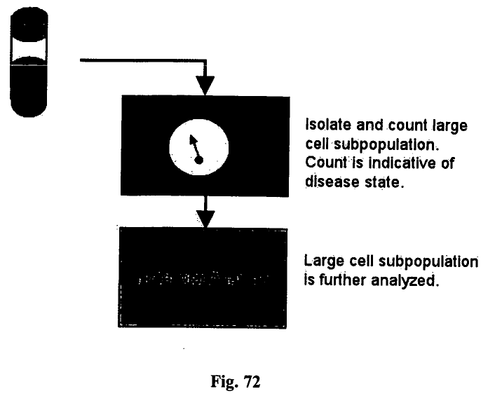

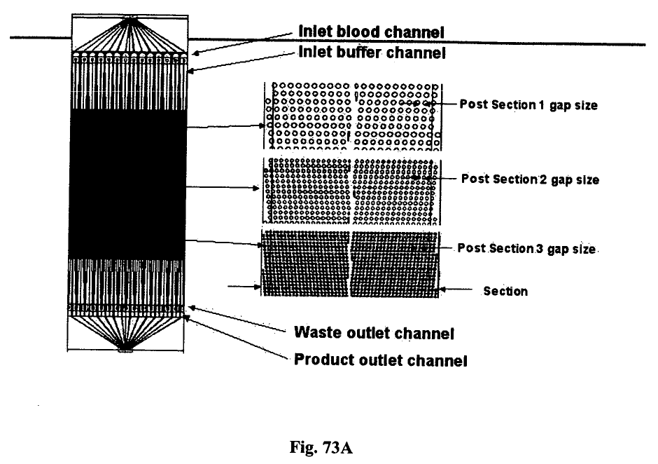

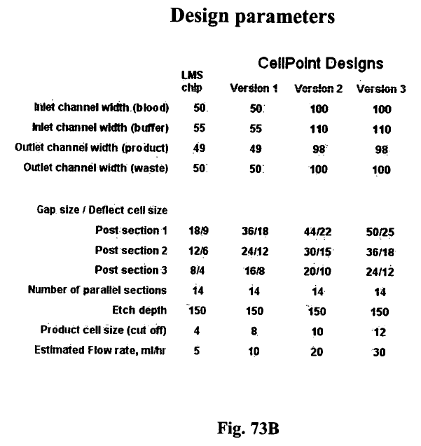

Using a deterministic device, therefore, it is possible to isolate a subpopulation of cells from blood or other bodily fluids based on size, which conveniently allows for the elimination of a large proportion of native blood cells when large cell types are targeted. As shown schematically in FIG. 72, a deterministic device may include counting means to determine the number of cells in the enriched sample, and further analysis of the cells in the enriched sample may provide additional information that is useful for diagnostic or other purposes.

### Example 15

### Enrichment of Fetal Nucleated Red Blood Cells from Maternal Blood

For this example, the device includes a deterministic separation component, as described herein, capable of separated fetal nucleated red blood cells and maternal white blood cells from maternal enucleated red blood cells. The deterministic component is connected to a reservoir containing sodium nitrite. A maternal blood sample, e.g., that has been diluted, is introduced into the device to produce a fraction enriched in fetal red blood cells and depleted of maternal red blood cells. This sample is directed into the reservoir where the sodium nitrite oxidizes the fetal heme iron, thereby increasing the magnetic responsiveness of the fetal red blood cells. A magnetic field is then applied, e.g., via a MACS column, and the altered fetal red blood cells bind to the magnet, while maternal white blood cells are not bound by the magnet. Removing the white blood cells, e.g., by a rinse, and then eliminating the magnetic field allows recovery of the fetal red blood cells, e.g., for analysis, storage, or further manipulation.

### Example 16

### Separation of Fetal Nucleated Red Blood Cells from Blood Using a High-Gradient Magnet

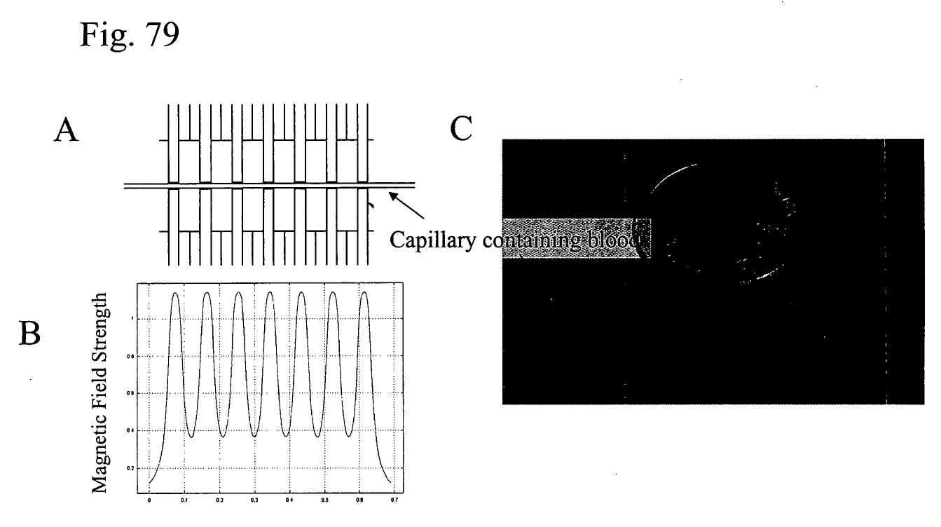

An exemplary high-gradient magnet useful for attracting red blood cells containing methemoglobin is shown schematically in FIG. 78. Red bloods placed in a capillary are concentrated to discrete regions because of the non-uniform nature of the applied magnetic field (FIGS. 79A-79C).

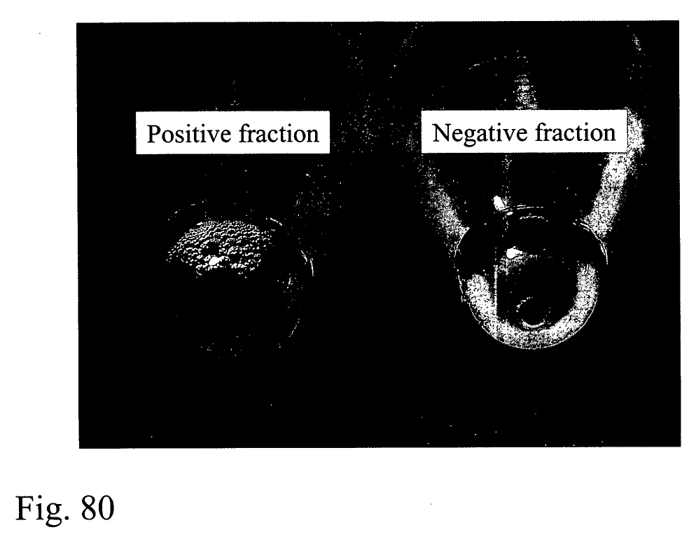

FIG. 80 is a picture of a pellet of nucleated red blood cells (positive fraction) and a pellet of white blood cells (negative fraction) prepared from male cord blood. Nucleated cells are first extracted from the blood using a deterministic lateral separation device, and treated with sodium nitrite at 50M for 10 min. The nucleated cells are then passed through a magnetic column where nucleated red blood cells are retained. In the column, the magnetic field strength is about 1 Tesla, the magnetic field gradient is about 3000 Tesla/m, and the flow velocity is about 0.4 mm/sec. White blood cells are rinsed out of the column using Dulbecco PBS buffer with 1% BSA and 2 mM EDTA, and collected as the negative fraction. The nucleated red blood cells are eluted from the column using the same buffer at a flow velocity of 4 mm/s and collected as the positive fraction.

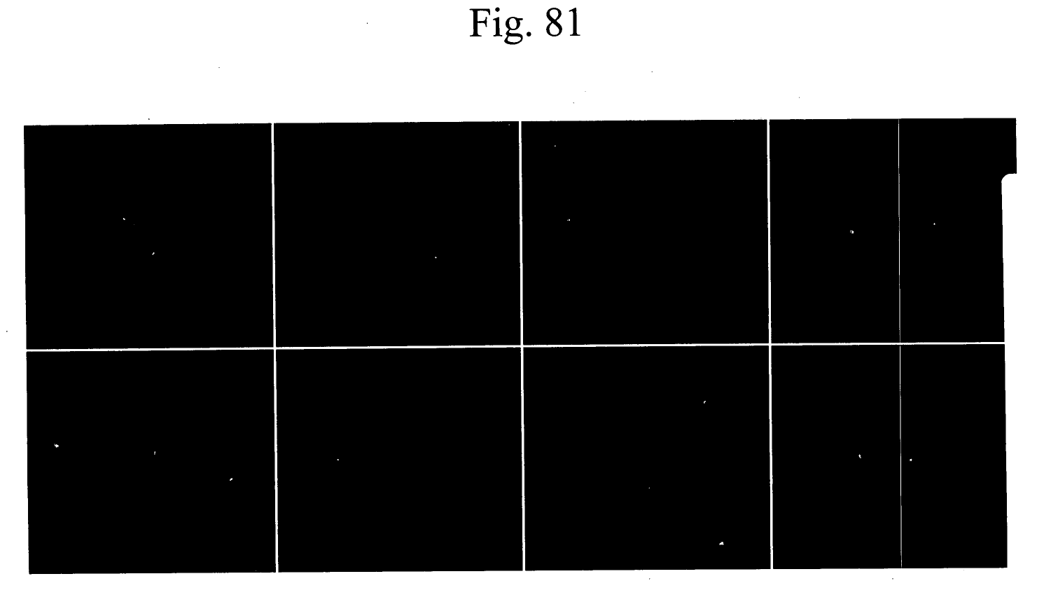

FIG. 81 is a series of fluorescence images of nucleated red blood cells isolated from maternal blood using the method described in FIG. 80. The cells are stained using fluorescence in situ hybridization (FISH). The X chromosome is identified with an aqua labeled probe for the alpha satellite region, while the Y chromosome is identified with red and green stains for the alpha satellite and satellite III regions, respectively. The nuclei are counterstained with DAPI (blue).

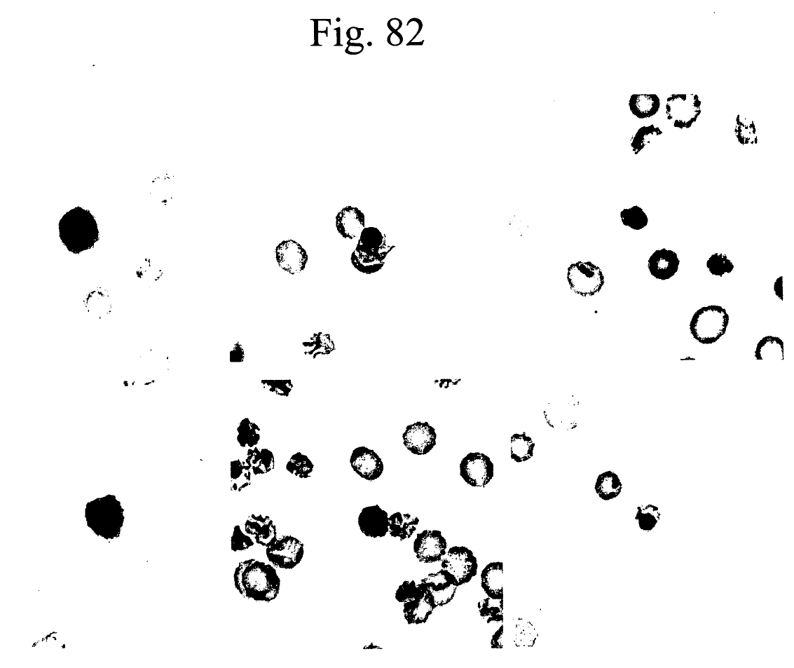

FIG. 82 shows nucleated red blood cells in different maturation stages isolated from maternal blood using the method described in FIG. 80. The cells are stained with Wright-Giemsa stain.

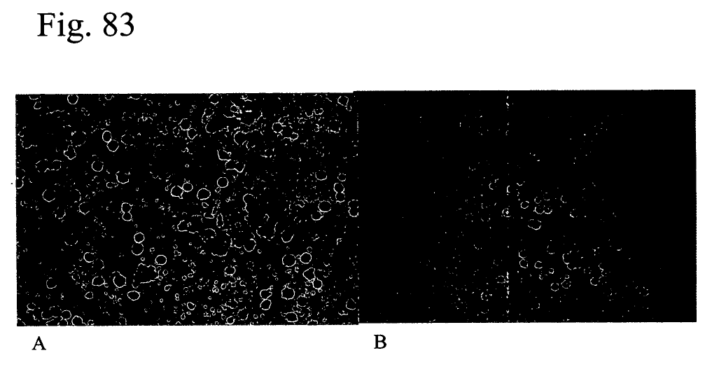

FIGS. 83A and 83B show micrographs of results of enrichment employing anti-CD71 antibodies (A) and the method described in FIG. 80 (B). The sample in A contained >200,000 nucleated cells from 1 mL of blood, while the sample in B contained about 100-500 nucleated cells per mL of blood. The purity of nucleated red blood cells obtained by the method described in FIG. 80 is about 1000 times better than antibody-based enrichment methods.

### Example 17

### Exemplary Methods for Enrichment of Cells

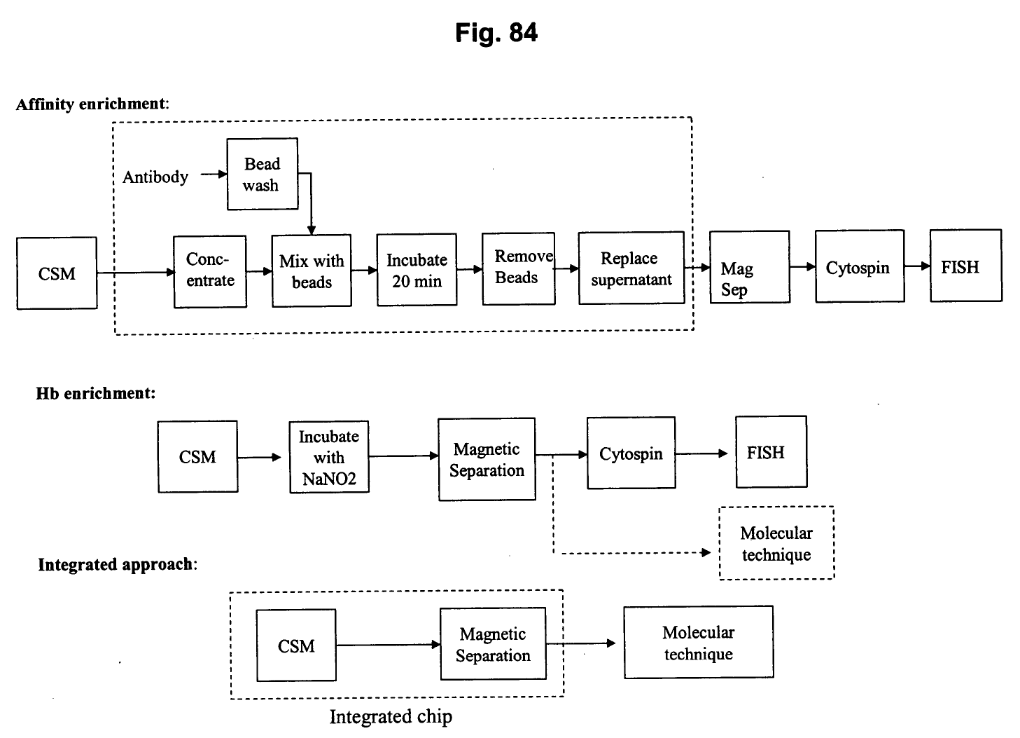

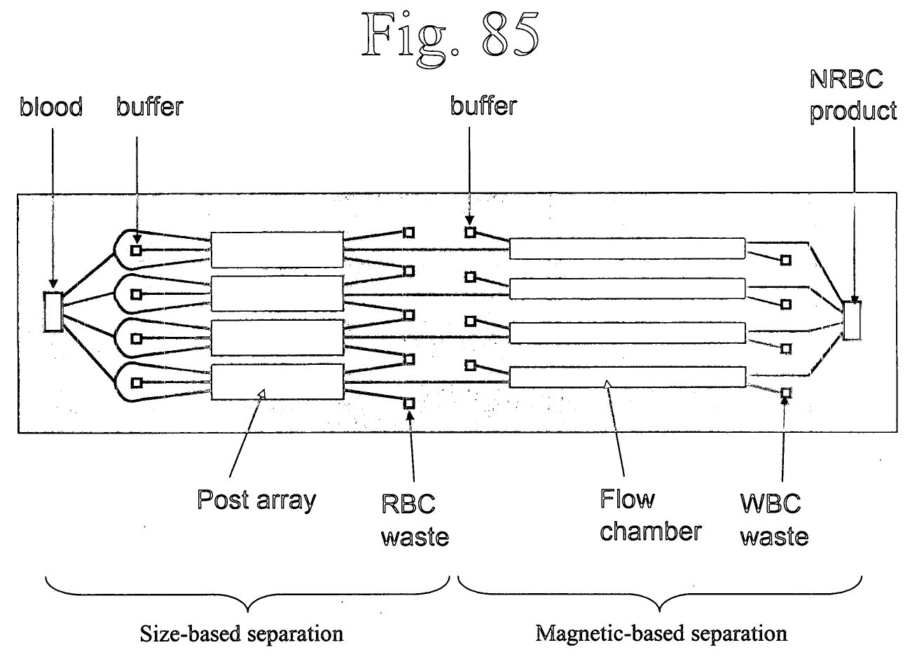

Three methods of implementing preferred embodiments of the invention are shown in FIG. 84. In affinity enrichment, a sample is passed through a deterministic device, as described herein. The output of the deterministic device is then contacted with magnetic beads coated with antibodies or other selective binding moieties. Cells bound to the beads are then magnetically separated, cytospun, and analyzed, e.g., by FISH. In hemoglobin enrichment, a sample is passed through a deterministic device, as described herein. The output of the deterministic device is then contacted with a reagent capable of oxidizing hemoglobin, e.g., sodium nitrite, and the magnetically responsive cells are magnetically separated. The separated cells may be cytospun and analyzed, e.g., via FISH, or may undergo molecular analysis. In the integrated approach, a device of the invention includes a deterministic enrichment component and a magnetic enrichment component, the output of which may be subjected to molecular analysis. FIG. 85 shows a schematic depiction of an integrated device of the invention.

### OTHER EMBODIMENTS

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A device for producing a sample enriched in a first cell or component thereof relative to a second component, said device comprising:
(a) a channel through which said first cell or component flows; and
(b) a magnet that produces a magnetic field of between 0.05 and 5.0 Tesla and a magnetic field gradient of between 100 Tesla/m and 1,000,000 Tesla/m in said channel.

2. The device of claim 1, wherein said first cell or component is retained in said channel and said second component is not retained in said channel.

3. The device of claim 1, wherein said first cell or component is not retained in said channel and said second component is retained in said channel.

4. The device of claim 1, wherein said channel comprises first and second outlets, and said first cell or component thereof is directed into said first outlet, while said second component is directed into said second outlet.

5. The device of claim 1, further comprising an analytical module that enriches said first cell or component based on size, shape, deformability, or affinity.

6. The device of claim 5, wherein said analytical module comprises a first channel comprising a structure that deterministically deflects particles having a hydrodynamic size above a critical size in a direction not parallel to the average direction of flow in said structure, wherein said particles are said first cell or component or said second component.

7. The device of claim 1, further comprising a reagent capable of altering a magnetic property of said first cell or component or second component of said sample.

8. The device of claim 7, wherein said reagent alters the magnetic properties of a protein present in said first cell or component or said second component.

9. The device of claim 8, wherein said protein comprises iron.

10. The device of claim 9, wherein said protein is fetal hemoglobin, adult hemoglobin, methemoglobin, myoglobin, or a cytochrome.

11. The device of claim 8, wherein said reagent comprises sodium nitrite, carbon dioxide, oxygen, carbon monoxide, or nitrogen.

12. The device of claim 1, wherein said first cell is a blood cell.

13. The device of claim 1, wherein said first cell is a nucleated cell.

14. The device of claim 1, wherein said first cell is an enucleated cell.

15. The device of claim 1, wherein said blood cell is an adult nucleated red blood cell.

16. The device of claim 1, wherein said blood cell is a fetal nucleated red blood cell.

17. The device of claim 16, wherein said fetal nucleated red blood cell is from a fetus of less than 10 weeks of age.

18. The device of claim 1, wherein said first cell is mammalian, avian, reptilian, or amphibian.

19. The device of claim 1, wherein said component of said first cell is selected from the group consisting of nuclei, peri-nuclear compartments, nuclear membranes, mitochondria, chloroplasts, or cell membranes, lipids, polysaccharides, proteins, nucleic acids, viral particles, or ribosomes.

20. The device of claim 7, wherein said reagent causes expression or overexpression of a protein that is magnetic in said first cell or component or said second component.

21. The device of claim 20, wherein said reagent is capable of transfecting said first cell or said second component with a magnetically responsive protein.

22. The device of claim 7, wherein said reagent comprises a magnetic particle that binds to or is incorporated into said first cell or component or said second component.

23. The device of claim 1, further comprising a pump capable of producing a flow rate of greater than 50,000 cells or components thereof flowing into said channel per second.

24. The device of claim 1, wherein at least 90% of said first cell or component is retained in said device and at least 90% of said second component is not retained in said device.

25. A method for producing a sample enriched in a first cell or component thereof relative to a second component, said method comprising the steps of:
(a) introducing a sample comprising said first cell or component into the device of claim 1;
(b) allowing the passage of said first cell or component or said second component in said sample relative to the other to be altered based on a magnetic property, thereby producing said sample enriched in said first cell or component.

26. The method of claim 25, wherein said sample introduced into said device in step (a) is enriched for said first cell or component relative to a third component.

27. The method of claim 26, wherein, prior to step (a), said sample is contacted with an analytical module that enriches said first cell or component relative to said third component based on size, shape, deformability, or affinity.

28. The method of claim 27, wherein said analytical module comprises a first channel comprising a structure that deterministically deflects particles having a hydrodynamic size above a critical size in a direction not parallel to the average direction of flow in said structure, wherein said particles are said first cell or component or are said third component of said sample.

29. The method of claim 25, wherein said sample enriched in said first cell or component retains at least 70% of said first cells or components present in said sample.

30. The method of claim 25, wherein said sample enriched in said first cell or component is enriched by a factor of 100.

31. The method of claim 25, further comprising the step, prior to step (b), of contacting said sample with a reagent capable of altering a magnetic property of said first cell or component or second component.

32. The method of claim 31, wherein said reagent alters the magnetic properties of a protein present in said first cell or component or said second component.

33. The method of claim 32, wherein said protein is fetal hemoglobin, adult hemoglobin, methemoglobin, myoglobin, or a cytochrome.

34. The method of claim 32, wherein said reagent comprises sodium nitrite, carbon dioxide, or nitrogen.

35. The method of claim 25, wherein said first cell is a blood cell.

36. The method of claim 25, wherein said first cell is a nucleated cell.

37. The method of claim 25, wherein said first cell is an enucleated cell.

38. The method of claim 35, wherein said blood cell is an adult nucleated red blood cell.

39. The method of claim 35, wherein said blood cell is a fetal nucleated red blood cell.

40. The method of claim 39, wherein said fetal nucleated red blood cell is from a fetus of less than 10 weeks of age.

41. The method of claim 25, wherein said first cell is mammalian, avian, reptilian, or amphibian.

42. The method of claim 25, wherein said component of said first cell is nuclei, peri-nuclear compartments, nuclear membranes, mitochondria, chloroplasts, or cell membranes, lipids, polysaccharides, proteins, nucleic acids, viral particles, or ribosomes.

43. The method of claim 31, wherein said reagent causes expression or overexpression of a protein that is magnetic in said first cell or component or said second component.

44. The method of claim 31, wherein said reagent comprises a magnetic particle that binds to or is incorporated into said first cell or component or said second component.

45. The method of claim 25, wherein said sample enriched in said first cell or component comprises at least 90% of said first cell or component in said sample introduced in step (a) and less than 10% of said second component in said sample introduced in step (a).

46. The method of claim 25, wherein greater than 50,000 cells or components thereof flow into said channel per second.

47. A method of producing a sample enriched in a first cell or component thereof relative to a second component, said method comprising the steps of:
(a) contacting a sample comprising said first cell or component with a reagent that alters the magnetic properties of a protein expressed in said first cell or component or said second component of said sample to produce an altered sample;
(b) contacting said altered sample with a channel having a magnet positioned relative to said channel and producing a magnetic field and magnetic field gradient capable of altering the passage of said first cell or component or said second component relative to the other, thereby producing said sample enriched in said first cell or component.

48. The method of claim 47, wherein, prior to or after step (a), said sample comprising said first cell or component is enriched for said first cell or component relative to a third component.

49. The method of claim 48, wherein, prior to step (a), said sample is contacted with an analytical module that enriches said first cell or component relative to said third component based on size, shape, deformability, or affinity.

50. The method of claim 49, wherein said analytical module comprises a first channel comprising a structure that deterministically deflects particles having a hydrodynamic size above a critical size in a direction not parallel to the average direction of flow in said structure, wherein said particles are said first cell or component or are said third component of said sample.

51. The method of claim 47 wherein said sample enriched in said first cell or component retains at least 70% of said first cells or components present in said sample.

52. The method of claim 47, wherein said sample enriched in said first cell or component is enriched by a factor of 100.

53. The method of claim 47, wherein said reagent alters the magnetic properties of a protein present in said first cell or component or said second component.

54. The method of claim 53, wherein said protein is fetal hemoglobin, adult hemoglobin, methemoglobin, myoglobin, or a cytochrome.

55. The method of claim 53, wherein said reagent comprises sodium nitrite, carbon dioxide, or nitrogen.

56. The method of claim 47, wherein said first cell is a blood cell.

57. The method of claim 47, wherein said first cell is a nucleated cell.

58. The method of claim 47 wherein said first cell is an enucleated cell.

59. The method of claim 56, wherein said blood cell is an adult nucleated red blood cell.

60. The method of claim 56, wherein said blood cell is a fetal nucleated red blood cell.

61. The method of claim 60, wherein said fetal nucleated red blood cell is from a fetus of less than 10 weeks of age.

62. The method of claim 47, wherein said first cell is mammalian, avian, reptilian, or amphibian.

63. The method of claim 47, wherein said component of said first cell is nuclei, peri-nuclear compartments, nuclear membranes, mitochondria, chloroplasts, or cell membranes, lipids, polysaccharides, proteins, nucleic acids, viral particles, or ribosomes.

64. The method of claim 47, wherein said reagent causes expression or overexpression of a protein that is magnetic in said first cell or component or said second component.

65. The method of claim 47, wherein said reagent comprises a magnetic particle that binds to or is incorporated into said first cell or component or said second component.

66. The method of claim 47, wherein said sample enriched in said first cell or component comprises at least 90% of said first cell or component in said sample contacted in step (a) and less than 10% of said second component in said sample contacted in step (a).

67. The method of claim 47, wherein a magnet produces a magnetic field of between 0.05 and 5.0 Tesla and a magnetic field gradient of between 100 Tesla/m and 1,000,000 Tesla/m in said channel.

68. The method of claim 47, wherein greater than 50,000 cells or components thereof flow into said channel per second.

69. A method for enriching a first analyte from a fluid sample containing said first analyte relative to second and third analytes in said sample, said method comprising:
(a) performing a first enrichment step to enrich said first analyte from said fluid sample based on hydrodynamic size using a plurality of obstacles that direct said first analyte in a first direction and said second analyte in a second direction, and
(b) performing a second enrichment step to enrich said first analyte from said fluid sample based on an intrinsic or extrinsic magnetic property of said first or third analyte.

70. The method of claim 69, wherein said fluid sample is a blood sample.

71. The method of claim 69, wherein said fluid sample is a maternal blood sample.

72. The method of claim 69, wherein said one or more analytes are red blood cells.

73. The method of claim 69, wherein said one or more analytes are fetal red blood cells.

74. The method of claim 69, wherein each of said one or more analytes comprises fetal hemoglobin, adult hemoglobin, methemoglobin, myoglobin, or a cytochrome.

75. The method of claim 69, wherein said second enrichment step comprises applying a magnetic field to the product of said first enrichment step.

76. The method of claim 75, wherein said magnetic field attracts said first or third analyte.

77. The method of claim 75, wherein said magnetic field repulses said first or third analyte.

78. The method of claim 75, wherein said magnetic field alters the passage of said first analyte relative to said third analyte

79. The method of claim 75, wherein said magnetic field is between 0.5 and 5.0 Tesla.

80. The method of claim 75, wherein said second enrichment step further comprises applying a magnetic field gradient of between 100 Tesla/m and 1,000,000 Tesla/m.

81. The method of claim 69, further comprising the step of deoxygenating said first enrichment product.

82. The method of claim 81, wherein said deoxygenating step comprises contacting the product of said first enrichment step with CO, CO₂, N₂, or NaNO₂.

83. The method of claim 69, further comprising the step of paramagnetizing said first or third analyte.

84. The method of claim 69, further comprising the step of diamagnetizing said first or third analyte.

85. The method of claim 69, wherein said first enrichment step and said second enrichment step occur in series.

86. The method of claim 69, 1 wherein said first enrichment step comprises a plurality of hydrodynamic size-based enrichment steps that occur in series to one another.

87. The method of claim 69, wherein said first enrichment step comprises a plurality of hydrodynamic size-based enrichment steps that occur in parallel to one another.

88. The method of claim 69, wherein said second enrichment step comprises a plurality of enrichment steps that occur in parallel to one another.

89. The method of claim 69, wherein said first enrichment step occurs during sample flow through.

90. The method of claim 69, wherein said second enrichment step occurs during sample flow through.

91. The method of claim 69, wherein said second enrichment step is based on an intrinsic magnetic property.

92. The method of claim 69, wherein said second enrichment step is based on an extrinsic magnetic property.

93. The method of claim 69, wherein greater than 50,000 analytes are subjected to enrichment per second.

94. A system comprising a first module comprising (a) an array of obstacles that selectively directs one or more first analytes having a hydrodynamic size greater than a critical size in a first direction towards a first outlet and one or more second analytes having a hydrodynamic size smaller than said critical size in a second direction towards a second outlet;
(b) a second module comprising a channel for receiving said one or more first analytes from said first outlet; and (c) a magnet that generates a magnetic field and magnetic field gradient in said channel to alter passage of said one or more first analytes.

95. The system of claim 94, wherein said one or more second analytes comprise enucleated red blood cells.

96. The system of claim 94, wherein said one or more first analytes comprise nucleated red blood cells.

97. The system of claim 94, wherein said one or more first analytes comprise fetal nucleated red blood cells.

98. The system of claim 94, wherein said one or more first analytes comprise fetal hemoglobin, adult hemoglobin, methemoglobin, myoglobin, or a cytochrome.

99. The system of claim 94, further comprising a reservoir containing a deoxygenating agent coupled to said array of obstacles or said channel.

100. The system of claim 94, further comprising a reservoir containing a probe for specifically binding said one or more first analytes or components thereof.

101. The system of claim 100, wherein said probe is a nucleic acid probe or an antibody probe.

102. The system of claim 94, wherein said magnetic field is between 0.5 and 5.0 Tesla.

103. The system of claim 94, wherein said magnetic field gradient is between 100 Tesla/m and 1,000,000 Tesla/m.

104. The system of claim 94, wherein the passage of said one or more first analytes is altered based on an intrinsic magnetic property.

105. A system comprising (a) a flow-through channel comprising a two dimensional array of obstacles that selectively directs one or more first analytes having a hydrodynamic size greater than a critical size in a first direction towards a first outlet and one or more second analytes having a hydrodynamic size less than a critical size in a second direction towards a second outlet; and (b) a magnet that generates a magnetic field and magnetic field gradient to alter the passage of said one or more first analytes.

106. The system of claim 105, wherein said one or more first analytes comprise fetal nucleated red blood cells.

107. The system of claim 105, wherein the passage of said one or more first analytes is altered based on the presence of hemoglobin.

108. The system of claim 105, wherein said magnetic field is between 0.5 and 5.0 Tesla.

109. The system of claim 105, wherein said magnetic field gradient is between 100 Tesla/m and 1,000,000 Tesla/m.
